(19) United States
(12) Patent Application Publication
(10) Pub. No.: US 2020/0345845 A1
(43) Pub. Date: Nov. 5, 2020

(54) COMPOSITIONS AND METHODS RELATED TO CELL SYSTEMS FOR PENETRATING SOLID TUMORS

(71) Applicant: RUBIUS THERAPEUTICS, INC., Cambridge, MA (US)

(72) Inventors: Avak Kahvejian, Lexington, MA (US); Robert J. Deans, Riverside, CA (US); Jordi Mata-Fink, Happy Valley, OR (US); Sivan Elloul, Newton, MA (US); Nathan Dowden, Winchester, MA (US)

(21) Appl. No.: 16/844,564

(22) Filed: Apr. 9, 2020

Related U.S. Application Data
(63) Continuation of application No. 15/829,678, filed on Dec. 1, 2017, now abandoned.
(60) Provisional application No. 62/429,275, filed on Dec. 2, 2016.

(51) Int. Cl. A61K 39/395; A61K 35/18; A61P 35/00; A61K 47/68; C07K 16/28

(52) U.S. Cl. CPC A61K 39/39533; A61K 35/18; C12N 5/0641; A61K 47/6811; C07K 16/2887; A61P 35/00

(57) ABSTRACT

The present disclosure provides, e.g., compositions and method for treating cancers, e.g., solid tumors. In embodiments, the compositions comprise an erythroid cell expressing an exogenous polypeptide, e.g., a polypeptide that promotes penetration of the erythroid cell into the solid tumor.

### RELATED APPLICATIONS

This application is a continuation of U.S. application Ser. No. 15/829,678, filed Dec. 1, 2017, which claims priority to U.S. Ser. No. 62/429,275 filed Dec. 2, 2016, the contents of which are incorporated herein by reference in their entirety.

### BACKGROUND

Red blood cells have been considered for use as drug delivery systems, e.g., to degrade toxic metabolites or inactivate xenobiotics, and in other biomedical applications.

### SEQUENCE LISTING

The instant application contains a Sequence Listing which has been submitted electronically in ASCII format and is hereby incorporated by reference in its entirety. Said ASCII copy, created on Dec. 1, 2017, is named R2081-701810_SL.txt and is 223,355 bytes in size.

### SUMMARY OF THE INVENTION

The invention includes cell systems for treating cancer, e.g., by killing cancer cells and/or by stimulating an immune response to cancer. One challenge in treatment of solid tumors is that therapeutic agents, especially large agents such as cell therapies, sometimes fail to penetrate the tumor mass. This disclosure shows, among other things, that erythroid cells comprising a binding agent can be delivered to the vasculature, and then exit the vasculature and accumulate in solid tumors. The disclosure also shows that erythroid cells comprising an anti-cancer agent (e.g., antibody) can treat cancers that are resistant to the antibody alone. Thus, the disclosure provides, e.g., compositions and methods related to cell systems for penetrating solid tumors.

In some embodiments, the cell systems involve erythroid cells that express one or more exogenous polypeptides with targeting function, cancer cell killing function, immune checkpoint inhibition, and/or costimulation.

The disclosure provides, in some aspects, a method of treating a subject having a cancer (e.g., a cancer described herein), comprising administering to the subject a preparation comprising a plurality of erythroid cells (e.g., erythroid cells described herein), each erythroid cell comprising an anti-cancer agent (e.g., an anti-cancer agent described herein). The anti-cancer agent may be an agent that binds a tumor moiety (e.g., a polypeptide, e.g., an antibody, that binds a cell surface tumor moiety) and/or an agent that has an anti-tumor effect, e.g., an immunostimulatory cytokine, a tumor starvation enzyme (e.g. asparaginase, methionine gamma lyase, or serine dehydrogenase), a cytotoxic small molecule, radionuclide, chemotherapeutic, toxin, small molecule, protein therapeutic, cancer vaccine, checkpoint modulator, pro-apoptotic agent, complement-dependent cytotoxicity (CDC) stimulator, or a fragment or variant thereof. In embodiments, the immunostimulatory cytokine is a Type 1 cytokine or a Type 2 cytokine, or a fragment or variant thereof. In some embodiments, one agent can have both a tumor binding activity and an anti-tumor effect. In some embodiments, each erythroid cell of the plurality has a tumor binding agent and a different agent that has an anti-tumor effect, and the agents may be separate or linked, e.g., expressed as separate agents (e.g., separate polypeptides) or as linked sequences, e.g., fusions).

The present disclosure provides, in some aspects, a method of treating a subject having a resistant, e.g., a refractory or relapsed cancer, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to treat the cancer,
thereby treating the cancer.

The present disclosure provides, in some aspects, a method of delivering an agent, e.g., a binding agent, to a cell of a resistant, e.g., a refractory or relapsed, cancer in a subject, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen,
thereby delivering the agent to the cell of the resistant cancer.

The present disclosure also provides, in some aspects, a method of treating a subject having a treatment naïve resistant cancer, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to treat the cancer,
thereby treating the cancer.

The present disclosure also provides, in some aspects, a method of delivering an agent, e.g., a binding agent, to a cell of a treatment naïve resistant cancer in a subject, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen,
thereby delivering the agent to the cell of the treatment naïve resistant cancer.

The present disclosure also provides, in some aspects, a method of treating a subject having a cancer, wherein the cancer comprises an oncogenic mutation, e.g., a translocation that places a cell cycle gene under control of a constitutive promoter, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to treat the cancer,
thereby treating the cancer.

The present disclosure also provides, in some aspects, a method of delivering an agent, e.g., a binding agent, to a cell of a cancer in a subject, wherein the cancer comprises an oncogenic mutation, e.g., a translocation that places a cell cycle gene under control of a constitutive promoter, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen,
thereby delivering the agent to the cell of the cancer.

The present disclosure also provides, in some aspects, a method of treating a subject having a B cell cancer, or of delivering an agent to a cancerous B cell in a subject, wherein the subject has developed resistance to an antibody selected from Table 1, e.g., an anti-CD20 antibody, comprising:
administering to the subject a preparation of cells, e.g., erythroid cells (e.g., enucleated erythroid cells) comprising a fusion protein comprising a transmembrane domain and a binding domain that binds a tumor antigen (e.g., wherein the binding domain is an anti-CD20 antibody domain), wherein:
a) the number of fusion proteins on the outer surface of the engineered erythroid cell is greater than 10⁴, e.g., greater than 2×10⁴, 3×10⁴, 4×10⁴, 5×10⁴, 6×10⁴, 7×10⁴, 8×10⁴, 9×10⁴ or greater than 10⁵, e.g., greater than 2×10⁵, 3×10⁵, 4×10⁵, 5×10⁵, 6×10⁵, 7×10⁵, 8×10⁵, 9×10⁵, 1×10⁶, 2×10⁶, 5×10⁶, or 1×10⁷ (and optionally up to 1×10⁷ or 1×10⁸), or about 1×10⁴-3×10⁴, 1×10⁴-5×10⁴, 1×10⁴-7×10⁴, 1×10⁴-9×10⁴, or
b) the number of fusion proteins on the outer surface of the engineered erythroid cell or the affinity of the binding domain for the tumor antigen is sufficient to induce:
   i) clustering of a tumor antigen, e.g., an antigen described herein, e.g., an antigen of Table 1, e.g., CD20 molecules on the surface of target cancer cells, e.g., B cells,
   ii) hyper-clustering of a tumor antigen, e.g., an antigen described herein, e.g., an antigen of Table 1, e.g., CD20, on the surface of target cancer cells, e.g., B cells,
   iii) cross-linking of a tumor antigen, e.g., an antigen described herein, e.g., an antigen of Table 1, e.g., CD20, on the surface of target cancer cells, e.g., B cells,
   iv) hyper cross-linking of a tumor antigen, e.g., an antigen described herein, e.g., an antigen of Table 1, e.g., CD20, on the surface of target cancer cells, e.g., B cells,
   v) apoptosis, e.g., caspase-independent apoptosis and/or caspase-dependent apoptosis, of target cancer cells, e.g., B cells,
   vi) activation of Src family tyrosine kinase, clustering of Fas molecule, inhibition or downregulation of BCL2, and/or inhibition or downregulation of BCLxl, in target cancer cells, e.g., B cells, or a combination thereof,
thereby treating the subject or delivering the agent to the cancerous B cell.

The present disclosure also provides, in some aspects, a cell, e.g., an erythroid cell (e.g., enucleated erythroid cell) comprising a fusion protein comprising a transmembrane domain and a binding domain that binds a tumor antigen (e.g., wherein the binding domain is an anti-CD20 antibody domain or an anti-PD-L1 antibody domain) wherein:
a) the number of fusion proteins on the outer surface of the erythroid cell is greater than 10⁴, e.g., greater than 2×10⁴, 3×10⁴, 4×10⁴, 5×10⁴, 6×10⁴, 7×10⁴, 8×10⁴, 9×10⁴ or greater than 10⁵, e.g., greater than 2×10⁵, 3×10⁵, 4×10⁵, 5×10⁵, 6×10⁵, 7×10⁵, 8×10⁵, 9×10⁵, 1×10⁶, 2×10⁶, 5×10⁶, or 1×10⁷ (and optionally up to 1×10⁷ or 1×10⁸), or about 1×10⁴-3×10⁴, 1×10⁴-5×10⁴, 1×10⁴-7×10⁴, 1×10⁴-9×10⁴, or
b) the number of fusion proteins on the outer surface of the erythroid cell or the affinity of the binding domain for the tumor antigen is sufficient to induce:
   i) clustering of a tumor antigen, e.g., an antigen described herein, e.g., an antigen of Table 1, e.g., CD20 molecules on the surface of target cancer cells, e.g., B cells,
   ii) hyper-clustering of a tumor antigen, e.g., an antigen described herein, e.g., an antigen of Table 1, e.g., CD20, on the surface of target cancer cells, e.g., B cells,
   iii) cross-linking of a tumor antigen, e.g., an antigen described herein, e.g., an antigen of Table 1, e.g., CD20, on the surface of target cancer cells, e.g., B cells,
   iv) hyper cross-linking of a tumor antigen, e.g., an antigen described herein, e.g., an antigen of Table 1, e.g., CD20, on the surface of target cancer cells, e.g., B cells,
   v) apoptosis, e.g., caspase-independent apoptosis and/or caspase-dependent apoptosis, of target cancer cells, e.g., B cells,
   vi) activation of Src family tyrosine kinase, clustering of Fas molecule, inhibition or downregulation of BCL2, and/or inhibition or downregulation of BCLxl, in target cancer cells, e.g., B cells, or a combination thereof.

The present disclosure also provides, in certain aspects, a method of treating a vascularized solid tumor in a subject comprising:
administering to the subject's bloodstream a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to treat the vascularized solid tumor
thereby treating the vascularized solid tumor.

The present disclosure also provides, in some aspects, a method of delivering an agent, e.g., a binding agent to a tumor cell of a vascularized solid tumor in a subject comprising:
administering to the subject's bloodstream a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to deliver the binding agent to a tumor cell of the vascularized solid tumor,
thereby delivering the agent, e.g., binding agent to the tumor cell of the vascularized solid tumor.

The present disclosure also provides, in some aspects, a method of enriching an anti-cancer agent, e.g., an anti-cancer antibody, at a solid tumor in a subject, or treating a solid tumor in the subject, comprising:
administering to the subject's bloodstream a preparation comprising a plurality of cells, e.g., erythroid cells (e.g., enucleated erythroid cells), each cell of the plurality comprising the anti-cancer agent, in an amount sufficient to treat the solid tumor,
wherein optionally the anti-cancer agent comprises an exogenous polypeptide comprising a binding agent, e.g., an antibody, against a tumor cell antigen,
thereby enriching an anti-cancer agent or treating the solid tumor.

The present disclosure also provides, in some aspects, a method of enriching an anti-cancer agent, e.g., an anti-cancer antibody, at a solid tumor in a subject, or treating a solid tumor in the subject, comprising:
administering to the subject's bloodstream a preparation comprising a plurality of cells, e.g., erythroid cells, each cell of the plurality comprising the anti-cancer agent, in an amount sufficient to treat the solid tumor,
wherein the anti-cancer agent comprises an exogenous polypeptide comprising a binding agent, e.g., an antibody, against a tumor cell antigen,
thereby enriching an anti-cancer agent or treating the solid tumor.

The disclosure further provides, in certain aspects, a method of delivering an erythroid cell to an extravascular site in a subject, comprising:
administering to the subject's bloodstream an effective amount of a preparation comprising a plurality of cells, e.g., erythroid cells (e.g., enucleated erythroid cells), each cell of the plurality comprising, on its surface, an exogenous polypeptide comprising a binding agent, e.g., an antibody, against an antigen present at the extravascular site, e.g., a tumor antigen,
thereby delivering the erythroid cell to the extravascular site.

The disclosure further provides, in certain aspects, a method of delivering an agent, e.g., a binding agent, to an extravascular site in a subject, comprising:
administering to the subject's bloodstream an effective amount of a preparation comprising a plurality of cells, e.g., erythroid cells (e.g., enucleated erythroid cells), each cell of the plurality comprising, on its surface, an exogenous polypeptide comprising a binding agent, e.g., an antibody, against an antigen present at the extravascular site, e.g., a tumor antigen,
thereby delivering the agent, e.g., binding agent, to the extravascular site.

The present disclosure also provides, in some aspects, a method of treating a non-vascularized, e.g., a prevascularized, solid tumor in a subject comprising:
administering to the subject's bloodstream a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to treat the non-vascularized solid tumor
thereby treating a prevascularized solid tumor.

The present disclosure also provides, in some aspects, a method of delivering an agent, e.g., a binding agent to a tumor cell of a non-vascularized, e.g., prevascularized, solid tumor in a subject comprising:
administering to the subject's bloodstream a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen, in an amount sufficient to deliver the binding agent to a tumor cell of the non-vascularized solid tumor,
thereby delivering the agent to the tumor cell of the non-vascularized solid tumor.

The present disclosure also provides, in some aspects, a method of treating a subject having a cancer, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen,
wherein the density of binding agents on the surface of the erythroid cell is sufficient that, upon binding of the binding agents with tumor cell antigen, a tumor antigen accumulates in a lipid raft, the distribution of the antitumor cell antigen is sufficiently perturbed to alter signalling in the tumor cell, the density of tumor antigens on the surface of the cancer cell is significantly altered, an anti-apoptotic pathway (e.g., BCL2 and/or BCLxl pathway) is inhibited, an apoptotic pathway of the cancer cell is induced, a necrotic pathway of the cancer cell is induced, or the membrane properties of the cancer cell are significantly altered, or a combination thereof
thereby treating the cancer.

The present disclosure also provides, in some aspects, a cell, e.g., an erythroid cell (e.g., enucleated erythroid cell) comprising an exogenous polypeptide comprising a binding agent, e.g., an antibody, against a tumor cell antigen,
wherein the density of binding agents on the surface of the erythroid cell is sufficient that, upon binding of the binding agents with tumor cell antigen, a tumor antigen accumulates in a lipid raft, the distribution of the antitumor cell antigen is sufficiently perturbed to alter signalling in the tumor cell, the density of tumor antigens on the surface of the cancer cell is significantly altered, an anti-apoptotic pathway (e.g., BCL2 and/or BCLxl pathway) is inhibited, an apoptotic pathway of the cancer cell is induced, a necrotic pathway of the cancer cell is induced, or the membrane properties of the cancer cell are significantly altered, or a combination thereof

The present disclosure also provides, in some aspects, a method of treating a tumor, e.g., a vascularized tumor, in a subject comprising:
administering (e.g., to the subject's bloodstream) to the subject an erythroid cell (e.g., enucleated erythroid cell) comprising, e.g., on its surface,
a moiety that interferes with the ability of an immune checkpoint-ligand (e.g., PD-L1) to functionally engage an immune checkpoint molecule (e.g., PD1), e.g., an immune checkpoint molecule expressed on a tumor infiltrating lymphocyte,
wherein if the moiety is expressed as a fusion protein:
i) at least 50, 60, 70, 80, 90, 95, or 99% of the fusion proteins on the surface of the erythroid cell have an identical sequence,
ii) at least 50, 60, 70, 80, 90, 95, or 99% of the fusion protein have the same transmembrane region,
iii) the fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;
iv) at least 50, 60, 70, 80, 90, 95, or 99% of the fusion proteins do not differ from one another by more than 1, 2, 3, 4, 5, 10, 20, or 50 amino acids,
v) the moiety lacks a sortase transfer signature (i.e., a sequence that can be created by a sortase reaction) such as LPXTG,
vi) the moiety is present on less than 1, 2, 3, 4, or 5 sequence-distinct fusion polypeptides;
vii) the moiety is present on a single fusion polypeptide;
viii) the fusion protein does not contain Gly-Gly at the junction of an endogenous transmembrane protein and the moiety; or
ix) the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof,
thereby treating the tumor.

The present disclosure also provides, in some aspects, an erythroid cell comprising, e.g., on its surface, a moiety that interferes with the ability of an immune checkpoint-ligand (e.g., PD-L1) to functionally engage an immune checkpoint molecule (e.g., PD1), e.g., an immune checkpoint molecule expressed on a tumor infiltrating lymphocyte,
wherein if the moiety is expressed as a fusion protein:
i) at least 50, 60, 70, 80, 90, 95, or 99% of the fusion proteins on the surface of the erythroid cell have an identical sequence,
ii) at least 50, 60, 70, 80, 90, 95, or 99% of the fusion protein have the same transmembrane region,
iii) the fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;
iv) at least 50, 60, 70, 80, 90, 95, or 99% of the fusion proteins do not differ from one another by more than 1, 2, 3, 4, 5, 10, 20, or 50 amino acids,
v) the moiety lacks a sortase transfer signature,
vi) the moiety is present on less than 1, 2, 3, 4, or 5 sequence-distinct fusion polypeptides;
vii) the moiety is present on a single fusion polypeptide;
viii) the fusion protein does not contain Gly-Gly at the junction of an endogenous transmembrane protein and the moiety; or
ix) the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment, or a combination thereof.

The present disclosure also provides, in some aspects, a method of treating a tumor, e.g., a vascularized tumor, in a subject comprising:
administering (e.g., to the subject's bloodstream) to the subject an erythroid cell (e.g., enucleated erythroid cell) comprising, e.g., on its surface, a stimulatory molecule, e.g., a costimulatory molecule, e.g., 4-1BBL or a fragment thereof, wherein
the level of the stimulatory molecule, e.g., costimulatory molecule or the affinity of the stimulatory molecule, e.g., costimulatory molecule for a binding partner on an immune cell (e.g., T cell) is sufficient to: induce immune cell proliferation, increase secretion of a cytokine (e.g., IL2 or IFN-gamma), or reduce activation-induced cell death in an immune cell, e.g., tumor infiltrating lymphocyte and/or T cell,
wherein if the moiety is expressed as a fusion protein:
i) at least 50, 60, 70, 80, 90, 95, or 99% of the fusion proteins on the surface of the erythroid cell have an identical sequence,
ii) at least 50, 60, 70, 80, 90, 95, or 99% of the fusion protein have the same transmembrane region,
iii) the fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;
iv) at least 50, 60, 70, 80, 90, 95, or 99% of the fusion proteins do not differ from one another by more than 1, 2, 3, 4, 5, 10, 20, or 50 amino acids,
v) the moiety lacks a sortase transfer signature,
vi) the moiety is present on less than 1, 2, 3, 4, or 5 sequence-distinct fusion polypeptides;
vii) the moiety is present on a single fusion polypeptide;
viii) the fusion protein does not contain Gly-Gly at the junction of an endogenous transmembrane protein and the moiety; or
ix) the fusion protein does not contain Gly-Gly, or the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof,
thereby treating the tumor.

The present disclosure also provides, in some aspects, an erythroid cell comprising, e.g., on its surface, a stimulatory molecule, e.g., a costimulatory molecule, e.g., 4-1BBL or a fragment thereof, wherein
the level of the stimulatory molecule, e.g., costimulatory molecule or the affinity of the stimulatory molecule, e.g., costimulatory molecule for a binding partner on an immune cell (e.g., T cell) is sufficient to: induce immune cell proliferation, increase secretion of a cytokine (e.g., IL2 or IFN-gamma), or reduce activation-induced cell death in an immune cell, e.g., tumor infiltrating lymphocyte and/or T cell,
wherein if the moiety is expressed as a fusion protein:
i) at least 50, 60, 70, 80, 90, 95, or 99% of the fusion proteins on the surface of the erythroid cell have an identical sequence,
ii) at least 50, 60, 70, 80, 90, 95, or 99% of the fusion protein have the same transmembrane region,
iii) the fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;
iv) at least 50, 60, 70, 80, 90, 95, or 99% of the fusion proteins do not differ from one another by more than 1, 2, 3, 4, 5, 10, 20, or 50 amino acids,
v) the moiety lacks a sortase transfer signature,
vi) the moiety is present on less than 1, 2, 3, 4, or 5 sequence distinct fusion polypeptides;
vii) the moiety is present on a single fusion polypeptide;
viii) the fusion protein does not contain Gly-Gly at the junction of an endogenous transmembrane protein and the moiety;
ix) the fusion protein does not contain Gly-Gly, or the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof.

The present disclosure also provides, in some aspects, a method of stimulating an immune effector cell, e.g., a T cell, in a subject, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a costimulatory molecule (e.g., 4-1BB-L, OX40-L, GITR-L, or ICOS-L), in an amount sufficient to stimulate the immune effector cell,
thereby stimulating the immune effector cell.

The present disclosure also provides, in some aspects, a method of detecting a cancer, e.g. a solid tumor, comprising:
administering (e.g., to the subject's bloodstream) to the subject a preparation comprising a plurality of cells, e.g., erythroid cells (e.g., enucleated erythroid cells), each cell of the plurality comprising, on its surface, an exogenous polypeptide comprising a binding agent, e.g., an antibody, against a tumor cell antigen,
wherein each cell in the plurality also comprises (e.g., on the cell surface on inside the cell) a label detectable by in vivo imaging, e.g., a radionuclide, fluorophore, bioluminescent agent, or MRI contrast agent; and
detecting the label, e.g., by MRI or radiological detection,
thereby detecting the cancer.

The present disclosure also provides, in some aspects, an erythroid cell (e.g., enucleated erythroid cell) comprising:

on its surface, an exogenous polypeptide comprising a binding agent, e.g., an antibody, against a tumor cell antigen, and
a label detectable by in vivo imaging, e.g., a radionuclide, fluorophore, bioluminescent agent, or MRI contrast agent, (e.g., on the cell surface on inside the cell).

In some aspects, the present disclosure provides a method of delivering, presenting, or expressing an anti-cancer agent comprising providing an erythroid cell described herein.

In some aspects, the present disclosure provides a method of producing an erythroid cell (e.g., enucleated erythroid cell) described herein, providing contacting an erythroid cell precursor with one or more nucleic acids encoding the exogenous polypeptides and placing the cell in conditions that allow enucleation to occur.

In some aspects, the present disclosure provides a preparation, e.g., pharmaceutical preparation, comprising a plurality of erythroid cells (e.g., enucleated erythroid cells) described herein, e.g., at least 10⁸, 10⁹, 10¹⁰, 10¹¹, or 10¹² cells.

The following embodiments can apply to any of the aspects herein, e.g., any of the compositions and methods herein above.

In some embodiments, the agent is a therapeutic agent (e.g., an anti-cancer agent) or a diagnostic agent (e.g., a label detectable by in vivo imaging). In embodiments, the agent is promotes T cell activation, stimulation, or proliferation. In embodiments, the agent inhibits cancer cell growth or survival. In embodiments, the agent has two or more properties of agents described herein.

In some embodiments, the cell, e.g., erythroid cell, is autologous. In some embodiments, the cell is allogeneic.

In some embodiments, the cell system is administered in an amount and for a time effective to result in one of (or more, e.g., 2 or more, 3 or more, 4 or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

In embodiments, the tumor is non-metastatic. In embodiments, the tumor is metastatic. In embodiments, the tumor is vascularized and non-metastatic. In embodiments, the tumor comprises one or more tumor blood vessels. In embodiments, the tumor (e.g., vascularized tumor) secretes VEGF or bFGF. In embodiments, the tumor (e.g., non-vascularized tumor) does not produce VEGF or bFGF. In embodiments, the tumor (e.g., non-vascularized tumor) produces an anti-VEGF enzyme such as PGK. In embodiments, the tumor (e.g., vascularized tumor) does not produce an anti-VEGF enzyme such as PGK. In embodiments, the tumor comprises a necrotic region. In embodiments, the tumor expresses a pro-angiogenic factor.

In embodiments, the cancer expresses one or more tumor antigen herein, e.g., CD19, CD20, CD30, CD33, CD52, EGFR, GD2, HER2/neu, or VEGF, or a combination thereof.

In embodiments, the cancer is a cancer of Table 1 and the cancer antigen is a cancer antigen of Table 1. In embodiments, the cancer is a cancer of Table 3. In embodiments, the cancer lacks a functional caspase apoptosis pathway. In embodiments, the solid tumor is a solid tumor of Table 3. In embodiments, the solid tumor is a primary tumor or a metastatic tumor lesion. In embodiments, the location of the primary tumor is known. In embodiments, the cancer is other than a cancer of unknown primary.

In embodiments, after administration, the erythroid cells are resident or persistent in an extravascular region of the tumor, a non-necrotic region of the tumor, or an interstitial region of the tumor. In embodiments, an erythroid cell that is persistent in a tumor is resident in the tumor for at least 3, 6, or 12 hours or 1, 2, 3, 4, 5, 6, 7, 14, 21, or 28 days (e.g., up to 14 or 28 days).

In embodiments, the amount of binding agent on the surface of the erythroid cell is sufficient, or the binding affinity of the binding agent for its target is sufficient, or the erythroid cells are administered to the subject in an amount sufficient:
a) that the ratio of erythroid cells to tumor cells in the tumor (or in a 1, 2, 5, 10, 20, 50, 100, 200, 500, or 1000 mm³ region of the tumor) is at least about 100:1, 50:1, 20:1, 10:1, 5:1, 2:1, 1:1, 1:2, 1:5, 1:10, 1:20, 1:50, or 1:100 (e.g., up to about 100:1 or 10:1);
b) that the ratio of the erythroid cells to endogenous erythroid cells in the tumor is at least 2:1, 5:1, 10:1, 20:1, 50:1, 100:1, 1,000:1, or 10,000:1 (e.g., up to 100:1, 1,000:1 or 10,000:1);
c) to induce hypercrosslinking of a cancer cell surface protein, e.g., CD20;
d) that the ratio of erythroid cells resident in the tumor to the number of erythroid cells in the subject's bloodstream is greater than 1:1, 2:1, 3:1, 4:1, 5:1 10:1, 20:1, or 100:1 (and optionally up to 10:1 or 100:1), e.g., at least 1 hour or 12 hours or 1 day, 3, days, 5 days, or 7 days after administration of the cells or at the peak of erythroid cell accumulation in the tumor;
e) that the amount of binding agent, e.g., antibody, resident in the tumor (or in a 1, 2, 5, 10, 20, 50, 100, 200, 500, or 1000 mm³ region of the tumor) is at least 2, 3, 4, 5, 10, 20, 50, or 100-fold greater (and, optionally up to 10 or 100-fold greater) than the amount of an otherwise similar binding agent, e.g., antibody that is not associated with an erythroid cell, e.g., a free antibody;
f) to increase the erythroid cells' persistence in the tumor (e.g., by at least 2, 3, 4, 5, 10, 20, 50, 100, 200, or 500-fold), as compared with a reference, e.g., with a similar erythroid cell that lacks the binding agent;
g) that the concentration of erythroid cells in the tumor or a 1, 2, 5, 10, 20, 50, 100, 200, 500, or 1000 mm³ region of the tumor is enriched compared to an extratumoral compartment, e.g., at least 10, 20, 30, 40, 50, 60, 70, 80, 90, or 95% (and optionally up to 95% or 99%) of the erythroid cells in the subject are found in the tumor and less than 30, 20, 10, 9, 8, 7, 6, 5, 4, 3, 2, or 1% of the erythroid cells found in a second compartment, e.g., the liver; e.g., at least 1 hour or 12 hours or 1 day, 3, days, 5 days, or 7 days after administration of the cells or at the peak of erythroid cell accumulation in the tumor;
h) that the concentration of erythroid cells in a vasculature-adjacent region of the tumor (e.g., a 1, 2, 5, 10, 20, 50, 100, 200, 500, or 1000 mm³ region) is enriched compared to a vasculature-distant region of the tumor (e.g., a 1, 2, 5, 10, 20, 50, 100, 200, 500, or 1000 mm³ region), e.g., at least 10, 20, 30, 40, 50, 60, 70, 80, 90, or 95% (and optionally up to 95% or 99%) of the erythroid cells in the subject are found in the vasculature-adjacent region and less than 30, 20, 10, 9, 8, 7, 6, 5, 4, 3, 2, or 1%, or an undetectable amount, of the erythroid cells found in a vasculature-distant region; e.g., at least 1 hour or 12 hours or 1 day, 3, days, 5 days, or 7 days after administration of the cells or at the peak of erythroid cell accumulation in the tumor, wherein optionally the vascular adjacent region is within 0.1, 0.2, 0.5, 1, 2, 3, 4, or 5 cm (e.g., up to 5 cm) of a blood vessel and the vascular-distant region of the tumor is further than 2, 3, 4, 5, or 10 cm of a blood vessel;
j) that the number of erythroid cells in circulation is sufficiently low such that the patient does not experience an infusion reaction, severe mucocutaneous reaction, Hepatitis B virus reactivation, or progressive multifocal leukoencephalopathy, or a combination thereof;
k) that the erythroid cells are present and/or active in the tumor site at least 1, 2, 3, 4, 5, 6, 7, 8, 9, 10, 11, or 12 months (and optionally up to 6, 9 or 12 months) after they are administered to the subject; or
l) that cancer cells undergo cell death (e.g., ADCC or apoptosis, e.g., caspase-independent apoptosis) in the presence of the erythroid cells at a higher rate than cancer cells in the presence of the same amount of the same binding agent not comprised by an erythroid cell, or any combination thereof.

In embodiments, the number of erythroid cells comprising a binding agent on their surface in circulation is sufficiently low such that the patient does not experience a side effect that is associated with the free binding agent, e.g., an antibody not associated with an erythroid cell. In embodiments (e.g., wherein the binding agent binds CD20, e.g., wherein the binding agent comprises rituximab or a fragment or variant thereof), the number of erythroid cells in circulation is sufficiently low such that the patient does not experience an infusion reaction, severe mucocutaneous reaction, Hepatitis B virus reactivation, or progressive multifocal leukoencephalopathy, or a combination thereof. In embodiments (e.g., wherein the binding agent binds CD52, e.g., wherein the binding agent comprises alemtuzumab or a fragment or variant thereof), the number of erythroid cells in circulation is sufficiently low such that the patient does not experience a cytopenia, infusion reaction, an infection, or a combination thereof. In embodiments (e.g., wherein the binding agent binds HER2/neu, e.g., wherein the binding agent comprises ado-Trastuzumab or a fragment or variant thereof), the number of erythroid cells in circulation is sufficiently low such that the patient does not experience hepatotoxicity, liver failure, reductions in left ventricular ejection fraction, or embryo-fetal toxicity, or a combination thereof. In embodiments (e.g., wherein the binding agent binds HER2/neu, e.g., wherein the binding agent comprises Trastuzumab or a fragment or variant thereof), the number of erythroid cells in circulation is sufficiently low such that the patient does not experience Cardiomyopathy, Infusion Reaction, Pulmonary Toxicity, or embryo-fetal toxicity, or a combination thereof. In embodiments (e.g., wherein the binding agent binds EGFR, e.g., wherein the binding agent comprises nimotuzumab or a fragment or variant thereof), the number of erythroid cells in circulation is sufficiently low such that the patient does not experience myalgia, somnolence, disorientation, hematuria and elevated liver function enzymes, or a combination thereof. In embodiments (e.g., wherein the binding agent binds EGFR, e.g., wherein the binding agent comprises cetuximab or a fragment or variant thereof), the number of erythroid cells in circulation is sufficiently low such that the patient does not experience infusion reaction or cardiopulmonary arrest, or a combination thereof. In embodiments (e.g., wherein the binding agent binds VEGF, e.g., wherein the binding agent comprises bevacizumab or a fragment or variant thereof), the number of erythroid cells in circulation is sufficiently low such that the patient does not experience gastrointestinal perforation, surgery and wound healing complications, hemorrhage, or a combination thereof. In embodiments (e.g., wherein the binding agent binds CD33, e.g., wherein the binding agent comprises gemtuzumab or a fragment or variant thereof), the number of erythroid cells in circulation is sufficiently low such that the patient does not experience hypersensitivity reactions including anaphylaxis, infusion reactions, pulmonary events, hepatotoxicity, or a combination thereof. In embodiments (e.g., wherein the binding agent binds CD20, e.g., wherein the binding agent comprises ibritumomab or a fragment or variant thereof), the number of erythroid cells in circulation is sufficiently low such that the patient does not experience serious infusion reactions, cytopenia (e.g., prolonged and/or severe), or severe cutaneous and mucocutaneous reactions, or a combination thereof. In embodiments (e.g., wherein the binding agent binds CD20, e.g., wherein the binding agent comprises Tositumomab or a fragment or variant thereof), the number of erythroid cells in circulation is sufficiently low such that the patient does not experience serious allergic reaction or cytopenia (e.g., prolonged and/or severe), or a combination thereof. In embodiments (e.g., wherein the binding agent binds EGFR, e.g., wherein the binding agent comprises panitumumab or a fragment or variant thereof), the number of erythroid cells in circulation is sufficiently low such that the patient does not experience dermatologic toxicity. In embodiments (e.g., wherein the binding agent binds CD20, e.g., wherein the binding agent comprises of atumumab or a fragment or variant thereof), the number of erythroid cells in circulation is sufficiently low such that the patient does not experience Hepatitis B Virus reactivation or progressive multifocal leukoencephalopathy, or a combination thereof. In embodiments (e.g., wherein the binding agent binds CTLA-4, e.g., wherein the binding agent comprises ipilimumab or a fragment or variant thereof), the number of erythroid cells in circulation is sufficiently low such that the patient does not experience immune-mediated adverse reaction (e.g., enterocolitis, hepatitis, dermatitis, neuropathy, or endocrinopathy) or a combination thereof. In embodiments (e.g., wherein the binding agent binds CD30, e.g., wherein the binding agent comprises brentuximab or a fragment or variant thereof), the number of erythroid cells in circulation is sufficiently low such that the patient does not experience progressive multifocal leukoencephalopathy, or a combination thereof. In embodiments (e.g., wherein the binding agent binds Her2, e.g., wherein the binding agent comprises pertuzumab or a fragment or variant thereof), the number of erythroid cells in circulation is sufficiently low such that the patient does not experience left ventricular dysfunction or embryo-fetal toxicity, or a combination thereof. In embodiments (e.g., wherein the binding agent binds CD20, e.g., wherein the binding agent comprises obinutuzumab or a fragment or variant thereof), the number of erythroid cells in circulation is sufficiently low such that the patient does not experience Hepatitis B Virus reactivation or progressive multifocal leukoencephalopathy, or a combination thereof. In embodiments (e.g., wherein the binding agent binds PD-1, e.g., wherein the binding agent comprises nivolumab or a fragment or variant thereof), the number of erythroid cells in circulation is sufficiently low such that the patient does not experience immune-mediated events (e.g., pneumonitis, colitis, hepatitis, endocrinopathy, nephritis and renal dysfunction, skin adverse reactions, or encephalitis), infusion reaction, complications of allogeneic HSCT, or embryo-fetal toxicity, or a combination thereof. In embodiments (e.g., wherein the binding agent binds PD-1, e.g., wherein the binding agent comprises pembrolizumab or a fragment or variant thereof), the number of erythroid cells in circulation is sufficiently low such that the patient does not experience an immune-mediated adverse reaction (e.g., colitis, hepatitis, hypophysitis, nephritis, hyperthyroidism, hypothyroidism) or embryo-fetal toxicity, or a combination thereof. In embodiments (e.g., wherein the binding agent binds PDGF-R α, e.g., wherein the binding agent comprises olaratumab or a fragment or variant thereof), the number of erythroid cells in circulation is sufficiently low such that the patient does not experience infusion-related reaction or embryo-fetal toxicity, or a combination thereof. In embodiments (e.g., wherein the binding agent binds PD-L1, e.g., wherein the binding agent comprises atezolizumab or a fragment or variant thereof), the number of erythroid cells in circulation is sufficiently low such that the patient does not experience an immune-related adverse reaction (e.g., pneumonitis, hepatitis, colitis, endocrinopathy, myasthenic syndrome, myasthenia gravis, Guillain-Barré or meningoencephalitis, pancreatitis), ocular inflammatory toxicity, infection, infusion reaction, or embryo-fetal toxicity, or a combination thereof. In embodiments (e.g., wherein the binding agent binds SLAMF7, e.g., wherein the binding agent comprises elotuzumab or a fragment or variant thereof), the number of erythroid cells in circulation is sufficiently low such that the patient does not experience infusion reaction, infection, second primary malignancy, hHepatotoxicity, or a combination thereof. In embodiments (e.g., wherein the binding agent binds EGFR, e.g., wherein the binding agent comprises necitumumab or a fragment or variant thereof), the number of erythroid cells in circulation is sufficiently low such that the patient does not experience cardiopulmonary arrest, hypomagnesemia, or a combination thereof. In embodiments (e.g., wherein the binding agent binds CD38, e.g., wherein the binding agent comprises daratumumab or a fragment or variant thereof), the number of erythroid cells in circulation is sufficiently low such that the patient does not experience infusion reaction, neutropenia, or thrombocytopenia, or a combination thereof. In embodiments (e.g., wherein the binding agent binds VEGFR2, e.g., wherein the binding agent comprises ramucirumab or a fragment or variant thereof), the number of erythroid cells in circulation is sufficiently low such that the patient does not experience hemorrhage, arterial thromboembolic event, hypertension, infusion related reactions, gastrointestinal perforation, clinical deterioration in patients with cirrhosis, reversible posterior leukoencephalopathy syndrome, or a combination thereof. In embodiments (e.g., wherein the binding agent binds GD2, e.g., wherein the binding agent comprises dinutuximab or a fragment or variant thereof), the number of erythroid cells in circulation is sufficiently low such that the patient does not experience infusion reaction or neuropathy, or a combination thereof. In embodiments (e.g., wherein the binding agent binds CD19 and CD3, e.g., wherein the binding agent comprises blinatumomab or a fragment or variant thereof), the number of erythroid cells in circulation is sufficiently low such that the patient does not experience cytokine release syndrome or neurological toxicity, or a combination thereof. In embodiments (e.g., wherein the binding agent binds IL-6, e.g., wherein the binding agent comprises siltuximab or a fragment or variant thereof), the number of erythroid cells in circulation is sufficiently low such that the patient does not experience infusion related reaction or gastrointestinal perforation, or a combination thereof. In embodiments (e.g., wherein the binding agent binds RANK ligand, e.g., wherein the binding agent comprises denosumab or a fragment or variant thereof), the number of erythroid cells in circulation is sufficiently low such that the patient does not experience infection, dermatologic reaction, osteonecrosis of the jaw, or suppression of bone turnover, or a combination thereof. In embodiments (e.g., wherein the binding agent binds EpCAM and CD3, e.g., wherein the binding agent comprises catumaxomab or a fragment or variant thereof), the number of erythroid cells in circulation is sufficiently low such that the patient does not experience abdominal pain, pyrexia, fatigue, or nausea/vomiting, or a combination thereof.

In embodiments, at least about 1×10¹⁰, 2×10¹⁰, 5×10¹⁰, 1×10¹¹, 2×10¹¹, 5×10¹¹, (e.g., up to 1×10¹²), erythroid cells are administered to the subject.

In embodiments, exogenous polypeptide comprising a binding agent further comprises a transmembrane domain. In embodiments, the binding agent is an antibody, antibody fragment, single-chain antibody, scFv, or nanobody. In embodiments, the binding agent comprises an anti-CD20 antibody or an anti-PL-L1 antibody.

In embodiments, the binding agent is other than an anti-CD20 antibody, other than rituximab, or other than an antibody against a cancer stem cell antigen (e.g., CD44, CD47, MET, EpCam, Her2, EGFR, or CD19). In embodiments, the binding agent is other than an antibody against CD133, CD3, CD, CD16, CD19, CD20, CD56, CD44, CD24, or CD133.

In embodiments, the tumor cell antigen is a membrane protein, e.g., a membrane phosphoprotein.

In embodiments, the erythroid cells bind or associate with non-necrotic tumor cells with a greater affinity than an otherwise similar non-genetically engineered erythroid cell, e.g., having a Kd that is lower by at least 2, 5, 10, 20, 50, or 100-fold (and optionally by up to 10-fold or 100-fold).

In embodiments, the binding agent has targeting activity (e.g., causes erythroid cells to accumulate at the tumor at higher levels than otherwise similar erythroid cells that lack the binding agent) and has anti-cancer activity (e.g., causes cancer cell death, or slows tumor growth compared to an untreated cancer). In embodiments, the binding agent has targeting activity and does not have anti-cancer activity.

In embodiments, the anti-cancer agent described herein comprises one or more of an immunostimulatory cytokine, a tumor starvation enzyme (e.g. asparaginase, methionine gamma lyase, or serine dehydrogenase), a cytotoxic small molecule, radionuclide, chemotherapeutic, toxin, small molecule, protein therapeutic, checkpoint modulator, or pro-apoptotic agent, or a fragment or variant thereof. In embodiments, the immunostimulatory cytokine is a Type 1 cytokine or a Type 2 cytokine, or a fragment or variant thereof. In embodiments, the immunostimulatory cytokine is IL-2 or IL-12, or a fragment or variant thereof.

In embodiments, the erythroid cell further comprises a second agent. In embodiments, the second agent is an anti-cancer agent (e.g., a second anti-cancer agent) and/or a second exogenous polypeptide. In embodiments, the second agent comprises a radionuclide, chemotherapeutic, toxin, small molecule, or protein therapeutic. In embodiments, the second exogenous polypeptide comprises a checkpoint modulator, pro-apoptotic agent, or a tumor starvation enzyme (e.g., asparaginase). In embodiments, the second agent promotes an immune function, e.g., the second agent comprises a proinflammatory cytokine or an inhibitor of an immune checkpoint molecule.

In embodiments, the second agent shows improved safety or reduced side effects when comprised by the erythroid cell, compared to the same amount of an otherwise similar second agent not comprised by an erythroid cell.

In embodiments, the binding agent or second agent increases immune cell activity against cancer cells, recruits immune cells to the cancer, increases immune cell activation, increases immune cell resistance to immune checkpoint inhibition, or a combination thereof. In embodiments, the binding agent or second agent increases apoptosis. In embodiments, the binding agent or second agent modulates ion levels in the cancer cell, e.g., increases or decreases levels of a given ion, e.g., increases calcium levels. In embodiments, the binding agent or second agent modulates calcium channel activity in a tumor cell.

In some embodiments, the resistant cancer is resistant to an antibody therapeutic, e.g., an antibody therapeutic of Table 1. In some embodiments, the resistant cancer is resistant to a small molecule drug. In embodiments, the small molecule drug is a chemotherapeutic agent, e.g., a chemotherapeutic described herein. In embodiments, e.g., embodiments relating to resistant subjects, the subject has failed to respond to treatment with an antibody of Table 1. In embodiments, the subject is naïve to an antibody of Table 1. In embodiments, the antibody is an anti-CD20 antibody, e.g., rituximab. In embodiments, the antibody domain binds a target of Table 1. In embodiments, the antibody domain comprises CDRs or VR from Table 2, e.g., using the Kabat or Chothia definitions.

In embodiments, a method herein (e.g., a method of treating a resistant cancer), comprises administering to the subject a preparation of erythroid cells comprising a fusion protein comprising a transmembrane domain and an antibody domain, wherein the antibody domain binds to a tumor antigen. In embodiments, the cancer has become resistant to an antibody that binds that antigen. In embodiments, the antibody domain comprised by the erythroid cell has the same CDRs as, or differs from the CDRs by no more than 1, 2, 3, 4, or 5 mutations relative to, an antibody therapeutic to which the cancer is resistant, e.g., the patient has relapsed after treatment with that antibody therapeutic.

In embodiments, e.g., embodiments relating to resistant subjects, the patient comprises endogenous antibodies against the anti-cancer antibody therapeutic, e.g., the patient comprises HAMA (human anti-mouse antibodies). In embodiments, after treatment with the erythroid cells, the patient displays a reduced level of anti-drug antibodies, e.g., HAMA, compared to a pre-treatment level of anti-drug antibodies. In embodiments, the patient has an impaired caspase-dependent apoptosis pathway, e.g., has a mutation in a caspase, e.g., an initiator or executioner caspase. In embodiments, the mutation is in Caspase 2, Caspase 8, Caspase 9, Caspase 10, Caspase 3, Caspase 6, or Caspase 7. In embodiments, at least a subset of cancer cells do not have a mutation affecting (e.g., deletion of) a protein bound by the anti-cancer antibody therapeutic, e.g., CD20 or a target of Table 1.

In embodiments, e.g., embodiments involving treating cancers having an oncogenic mutation, the method comprises obtaining knowledge that the cancer has the mutation. In embodiments, the cancer comprises a second oncogenic mutation, e.g., mutation selected from Table 8.

In embodiments, the level of the exogenous polypeptide (e.g., a costimulatory molecule) or the affinity of the exogenous polypeptide for a binding partner on an immune cell (e.g., T cell) is sufficient to induce immune cell proliferation.

In embodiments, the erythroid cells have an osmotic fragility of less than 50% cell lysis at 0.3%, 0.35%, 0.4%, 0.45%, or 0.5% NaCl. In some embodiments, the enucleated erythroid cell has approximately the diameter or volume as a wild-type, untreated erythroid cell, e.g., a reticulocyte. In some embodiments, the erythroid cells comprise greater than 1%, 2%, 3%, 4%, 5%, 6%, 7%, 8%, 9% or greater than 10% fetal hemoglobin. In some embodiments, the enucleated erythroid cell has approximately the same phosphatidylserine content on the outer leaflet of its cell membrane as a wild-type, untreated erythroid cell.

In embodiments, the transmembrane domain comprises a transmembrane portion of glycophorin A (GPA), glycophorin B, glycophorin C, glycophorin D, kell, band 3, aquaporin 1, glut 1, kidd antigen protein, or rhesus antigen.

In embodiments, in embodiments involving immune checkpoint modulation, the moiety is an antibody of Table 4 an antibody that binds an antigen of Table 4. In embodiments, the moiety (e.g., moiety that binds to PD-L1) is an anti-PD-L1 antibody, or extracellular fragment of PD1. In embodiments, the moiety binds to an immune checkpoint-ligand (e.g., PD-L1).

In embodiments, a composition herein comprises, or a method comprises administering, a population of erythroid cells that lack the exogenous polypeptide, e.g., admixed together with the plurality of erythroid cells comprising the exogenous polypeptide. In embodiments, the composition comprises, or the method comprises administering, a population of cells that was transfected or transduced with less than 100% efficiency.

In embodiments, e.g., embodiments involving costimulatory molecules, the erythroid cell further comprises a targeting moiety, e.g., an exogenous polypeptide comprising a binding moiety. In embodiments, the binding moiety binds an antigen on an immune effector cell, e.g., a T cell or NK cell. In embodiments, the binding moiety binds an antigen on a tumor cell or present in the tumor microenvironment. In embodiments, the costimulatory molecule is 4-1BB-L, OX40-L, GITR-L, or ICOS-L.

In embodiments, the stimulatory molecule is a molecule that stimulates an immune effector cells, e.g., a T cell. In embodiments, the stimulatory molecule is a primary stimulant or a costimulatory molecule. In embodiments, the erythroid cell comprises a costimulatory molecule and a primary stimulant. In other embodiments, the erythroid comprises a costimulatory molecule and does not comprise a primary stimulant. In embodiments, the erythroid comprises a costimulatory molecule and does not comprise an exogenous primary stimulant.

In embodiments, e.g., embodiments involving resistant subjects, the number of fusion proteins on the outer surface of the engineered erythroid cell or the affinity of the binding domain for the tumor antigen is sufficient to induce binding of the erythroid cells to a target cell, e.g., a B cell.

In embodiments, the administration is systemic or local. In embodiments, the administration is to the bloodstream, e.g., intravenous administration, e.g., intravenous infusion. In embodiments, the administration is to a tumor.

In some embodiments, the erythroid cell comprises on its surface:
i) a first costimulatory molecule (e.g., a costimulatory molecule described herein, e.g., a costimulatory molecule of Table 5), and
ii) a second costimulatory molecule (e.g., a costimulatory molecule of Table 5), and
iii) optionally, a third or more costimulatory molecules (e.g., a costimulatory molecule of Table 5), and
iv) optionally, a targeting moiety that binds a tumor antigen.

In some embodiments, the erythroid cell comprises on its surface:
i) a first agent that binds a first immune checkpoint molecule (e.g., an agent that binds an immune checkpoint molecule described herein, e.g., an agent that binds an immune checkpoint molecule of Table 4, e.g., an antibody or other binding agent of Table 4), and
ii) a second agent that binds a second immune checkpoint molecule (e.g., an agent that binds an immune checkpoint molecule of Table 4, e.g., an antibody or other binding agent of Table 4), and
iii) optionally, a third or more agents that bind an immune checkpoint molecule (e.g., an immune checkpoint molecule of Table 4), and
iv) optionally, a targeting moiety that binds a tumor antigen.

In some embodiments, the erythroid cell comprises on its surface:
i) a costimulatory molecule (e.g., a costimulatory molecule described herein, e.g., a costimulatory molecule of Table 5, e.g., 4-1BBL), and
ii) an agent that binds an immune checkpoint molecule (e.g., an agent that binds an immune checkpoint molecule described herein, e.g., an agent that binds an immune checkpoint molecule of Table 4, e.g., an antibody or other binding agent of Table 4, e.g., anti-PD-L1), and
iii) optionally, a targeting moiety that binds a tumor antigen.

In embodiments, e.g., embodiments involving erythroid cells for in vivo imaging, the erythroid cell comprises a label comprising a PET isotope, e.g., 11C, 13N, 150, 18F, 64Cu, 62Cu, 1241, 76Br, 82Rb, 89Zr and 68Ga. In embodiments, the erythroid cell comprises a label comprising a bioluminescent agent, e.g., luciferase.

In some embodiments, the exogenous polypeptide(s) are encoded by one or more exogenous nucleic acid(s) that are not retained by the enucleated erythroid cell.

In embodiments, the erythroid cell promotes T cell proliferation, e.g., proliferation of CD4+ T cells, CD8+ T cells, or both of CD4+ T cells and CD8+ T cells, e.g., by at least about 2, 3, 4, 5, 6, 7, 8, or 10-fold, e.g., compared to a sample lacking erythroid cells, e.g., by a PBMC proliferation assay, e.g., an assay of Example 5. In embodiments, the erythroid cell promotes proliferation of CD8+ T cells more strongly than of CD4+ T cells, e.g., by a factor of at least 2-fold or 3-fold difference in fold increase over the same amount of time, e.g., 5 days. In some embodiments, the ratio of erythroid cells to PMBCs at the beginning of the assay is about 1:1.

In embodiments, the erythroid cell promotes cytokine secretion in a sample of T cells, e.g., secretion of IFNg, TNFa, or both of IFNg and TNFa, e.g., by a flow cytometry assay, e.g., an assay of Example 5. In embodiments, the erythroid cell promotes increased cytokine secretion, e.g., an increase of at least 2, 3, 4, or 5 fold compared to an otherwise similar cell sample treated with otherwise similar erythroid cells that lack the exogenous protein. In some embodiments, increased cytokine secretion is due at least in part to increased proliferation of cytokine-secreting cells (e.g., CD4+ T cells, CD8+ T cells, or both of CD4+ T cells and CD8+ T cells).

In embodiments, the erythroid cells (e.g., erythroid cells expressing an anti-PD-L1 antibody) are used to treat a cancer that expresses PD-L1. In some embodiments, the cancer cells are exposed to IFN-gamma, e.g., endogenous IFN-gamma, e.g., in an amount sufficient to increase PD-L1 expression in the tumor cells. In some embodiments, the tumor expresses at least 100,000, 125,000, 150,000, 200,000, 250,000, 300,000, 350,000, or 400,000 copies of PD-L1 per cell.

In related aspects, the disclosure provides a method comprising:
(a) acquiring information (e.g., directly or indirectly) about the presence or level of PD-L1 expression on a tumor, e.g., whether the tumor cell has a number of PD-L1 polypeptides per cell that is greater than a reference value, e.g., wherein the reference value is one of 100,000, 125,000, 150,000, 200,000, 250,000, 300,000, 350,000, or 400,000, and
(b) responsive to (a), selecting a treatment or administering a treatment comprising a plurality of enucleated erythroid cells described herein, e.g., an enucleated erythroid cell comprising on its surface an exogenous polypeptide comprising an anti-PD-L1 binding agent, e.g., an anti-PD-L1 antibody.

The cell systems described herein may be used in combination with another (one or more) anti-proliferative, anti-neoplastic or anti-tumor drug or treatment that is not part of the cell system. Such drugs or treatments include chemotherapeutic drugs, e.g., cytotoxic drugs (e.g., alkylating agents, antimetabolites, anti-tumor antibiotics, topoisomerase inhibitors, mitotic inhibitors, corticosteroids); cancer growth blockers such as tyrosine kinase inhibitors and proteasome inhibitors; T cell therapy (e.g., CAR-T cell therapy) (see, e.g., PMID: 26611350), Natural Killer (NK) cell immunomodulation (see, e.g., PMID: 26697006); and cancer vaccines (PMID: 26579225); other chemical drugs such as L-asparaginase and bortezomib (Velcade®). Hormone therapies (or anti-hormone therapies) may be used, e.g., for hormone-sensitive cancers.

The cell systems described herein may also be used in combination with non-drug therapies for cancer such as surgery, radiotherapy, or cryotherapy. In some cases, treatment methods of the invention may include a cell system described herein in combination with 2 or more other therapies or drugs, e.g., breast cancer may be treated with a combination of a cell system described herein in combination with surgery or radiotherapy and a chemotherapeutic cocktail or biologic (e.g., an anti-HER2 antibody).

The disclosure contemplates all combinations of any one or more of the foregoing aspects and/or embodiments, as well as combinations with any one or more of the embodiments set forth in the detailed description and examples.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, NCBI, and Entrez sequences referred to herein, e.g., in any Table herein, are incorporated by reference. Unless otherwise specified, the sequence accession numbers specified herein, including in any Table herein, refer to the database entries current as of Dec. 2, 2016. When one gene or protein references a plurality of sequence accession numbers, all of the sequence variants are encompassed.

### BRIEF DESCRIPTION OF THE DRAWINGS

FIG. 1 is a graph showing the level of apoptosis in four lymphoma cell lines treated with (from left to right), anti-CD20 antibody alone, anti-CD20 antibody “crosslinked” together with a secondary anti-isotype antibody, control RCT-HA-GPA (at 1:1 or 1:5), or RCT-antiCD20 (at 1:1 or 1:5) antibody alone.

FIG. 2 is a graph showing level of apoptosis in CD20+ lymphoma cells treated with RCT-antiCD20 or control RCT-HA-GPA in the presence or absence of a caspase-dependent apoptosis inhibitor (FMK). 1-1 and 1-5 indicate a 1:1 and 1:5 ratio, respectively, of lymphoma cells to RCTs.

FIG. 3 shows tissue sections of CD20+ tumors from mice treated with RCT-antiCD20 or control RCT-HA-GPA. H&E, Hematoxylin and eosin, detects nucleated cells, endogenous red blood cells, and engineered erythroid cells. CD31 staining detects endothelial vasculature. CD20 staining detects Ramos tumor cells. HA detects engineered erythroid cells.

FIG. 4 is a fluorescence image of a xenograft tumor in a mouse treated with control RCT-HA-GPA (top panel) or RCT-antiCD20 (bottom panel).

FIG. 5 is a graph showing rescue of IL-2 secretion by RCTs comprising immune checkpoint inhibitors. From left to right, the bars correspond to Jurkat (Jurkat cells alone, baseline), Jurkat/Z138 (Jurkat cells co-cultured with NHL Z138 cells, showing inhibition of IL-2 secretion), Jurkat/Z138/Untransduced RCTs (Jurkat cells co-cultured with NHL Z138 cells and untransduced control erythroid cells, showing IL-2 secretion is not rescued), Jurkat/Z138/atezo-flag RCTs (Jurkat cells co-cultured with NHL Z138 cells and RCT-antiPDL1, showing rescue of IL-2 secretion), Jurkat/Z138/Ipi-flag RCTs (Jurkat cells co-cultured with NHL Z138 cells and RCT-antiCTLA4, showing rescue of IL-2 secretion). The next five bars show low or no IL-2 secretion in the absence of Jurkat cells. The rightmost bar shows roughly baseline levels of IL-2 secretion when Jurkat cells are co-cultured with untransduced control erythroid cells.

FIG. 6 is a graph showing interferon-gamma secretion in a standard antigen recall assay. Leftmost bar, PBMC alone showed baseline interferon-gamma secretion. Next 5 bars, PBMC co-cultured with the indicated numbers of control RCT-HA-GPA cells showed interferon gamma secretion levels similar to baseline. Rightmost 5 bars, PBMC co-cultured with the indicated numbers of RCT-antiPD1 cells showed increased interferon gamma secretion levels.

FIG. 7 is a graph showing NFκB activation as indicated by luciferase activity measured in RLU when Jurkat cells are co-cultured with RCT-41BBL or untransduced control erythroid cells.

FIG. 8 is a graph showing NFκB activation resulting from erythroid cells having the indicated number of copies of 4-1BB-L per cell.

FIG. 9 is a graph showing NFκB activation resulting from the indicated number of 4-1BB-L RCT cells, compared to an anti-4-1BB antibody.

FIG. 10 is a pair of graphs showing, in the left panel, the fold change in CD4+ T cells, and in the right panel, the fold change in CD8+ T cells, induced by 4-1BB-L RCT cells, compared to an anti-4-1BB antibody.

FIG. 11 is a pair of graphs showing, in the left panel, the IFN-gamma secretion, and in the right panel, the TNF-alpha secretion, induced by 4-1BB-L RCT cells, compared to an anti-4-1BB antibody.

FIG. 12 is a time course showing tumor size in mice treated with 41BBL-RCT and an untreated control.

FIG. 13 shows the ratio of erythroid cells in the tumor to erythroid cells in blood vessels for erythroid cells having an anti-PD-L1 antibody or an isotype control antibody.

FIG. 14 is a chart summarizing approximate fold change levels in signalling, proliferation, cytokine levels, and antigen recall activity induced by the indicated RCTs.

FIG. 15 is a graph quantifying the binding of the indicated erythroid cells to the indicated tumor cells.

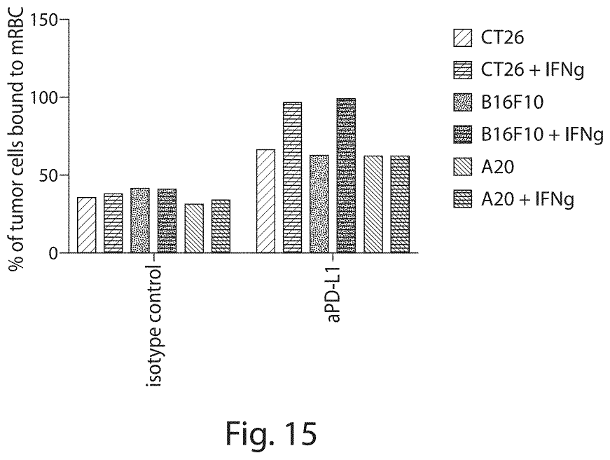

### DETAILED DESCRIPTION OF THE INVENTION

### Definitions

As used herein, the term “antibody” refers to a protein or part thereof, e.g., an immunoglobulin chain or fragment thereof, comprising at least one immunoglobulin variable domain sequence. The term “antibody” encompasses antibodies and antibody fragments. In an embodiment, an antibody is a multispecific antibody, e.g., a bispecific antibody. Examples of antibodies include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

As used herein, a “combination therapy” or “administered in combination” means that two (or more) different agents or treatments are administered to a subject as part of a treatment regimen for a particular disease or condition. The treatment regimen includes the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof.

Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

As used herein, “enucleated” refers to a cell that lacks a nucleus, e.g., a cell that lost its nucleus through differentiation into a mature red blood cell.

“Erythroid cells” as used herein, include nucleated red blood cells, red blood cell precursors, and enucleated red blood cells. For example, any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al., Mol Ther 2013, epub ahead of print September 3). In addition, the cells may be intended for autologous use or provide a source for allogeneic transfusion. In some embodiments, erythroid cells are cultured. In an embodiment an erythroid cell is an enucleated red blood cell.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell.

As used herein, “hyper cross-linking” of a tumor antigen refers to causing that antigen to cluster on the surface of the cell or redistribute into detergent-insoluble cell membrane complexes or signaling-processing centers. This redistribution may be assayed, e.g., as described in Polyak et al., “Identification of a Cytoplasmic Region of CD20 Required for Its Redistribution to a Detergent-Insoluble Membrane Compartment” The Journal of Immunology, Oct. 1, 1998, vol. 161 no. 7 3242-3248. Hyper cross-linking does not require the formation of covalent bonds between tumor antigens.

The term “resident”, as used herein, in reference to an agent being resident in a tumor, refers to the agent being present within the boundaries of the tumor, e.g., between tumor cells or inside a tumor cell.

The term “persistent” as used herein, in reference to an agent being persistent in a tumor, refers to an agent that is continuously resident in the tumor for a prolonged or predetermined time. For instance, if the agent comprises a targeting moiety that binds a tumor antigen, the agent is persistent in the tumor if the agent comprising the targeting moiety is continuously resident in the tumor for longer than an otherwise similar agent lacking the targeting moiety (e.g., an unmodified erythroid cell). In embodiments, an agent that is persistent in the tumor is resident in the tumor for at least 3, 6, or 12 hours or 1, 2, 3, 4, 5, 6, 7, 14, 21, or 28 days (e.g., up to 14 or 28 days).

A “resistant” tumor, as used herein, refers to a tumor that does not respond to a therapy. A resistant tumor may be a tumor in a subject who was treated with a therapy, and the tumor did not show a response to the therapy, e.g., the patient was refractory to the treatment, e.g., the patient exhibited progressive disease with no period of responsiveness. A resistant tumor may also be a tumor in a subject who was treated with a therapy and showed an initial response (e.g., complete response or partial response), but then the patient ceased to exhibit improvement (e.g., the patient's response plateaued) or relapsed (e.g., the patient exhibited progressive disease after the period of responsiveness). A resistant tumor may also be a treatment-naïve resistant tumor, e.g., a tumor in a subject who was not treated with the therapy, e.g., wherein the tumor was determined to be resistant to the therapy, e.g., using an in vitro assay of a tumor biopsy, or by tumor sequencing. A resistant tumor may also be a tumor, having a characteristic, e.g., a mutation, or a stage, which indicates it would be resistant to a therapy. In an embodiment, a therapeutic agent delivered by an erythroid cell described herein is more efficacious than the same therapeutic agent delivered freely, that is, not by an erythroid cell described herein, and is useful, e.g., to treat a tumor that is resistant to treatment with that therapeutic agent. In an embodiment, a therapeutic agent delivered by an erythroid cell described herein is administered to treat a tumor that is resistant to treatment with a different therapeutic agent. In an embodiment, treatment with the erythroid cell is initiated prior to resistance. In an embodiment, treatment with the erythroid cell is initiated after resistance.

A “refractory” tumor, as used herein, refers to a tumor that was treated with a therapy but did not show an adequate response, e.g., the patient is classified as having no response (NR) or progressive disease (PD) after the treatment. In an embodiment, a therapeutic agent delivered by an erythroid cell described herein is more efficacious than the same therapeutic agent delivered freely, that is, not by an erythroid cell described herein, and is useful, e.g., to treat a tumor that has is, or has become, refractory to that therapeutic agent. In an embodiment, a therapeutic agent delivered by an erythroid cell described herein is administered to treat a tumor that is, or has become, refractory to a different therapeutic agent. In an embodiment, treatment with the erythroid cell is initiated prior to the tumor becoming refractory. In an embodiment, treatment with the erythroid cell is initiated after the tumor becomes refractory.

A “relapsed” tumor, as used herein, refers to a tumor that went into remission (e.g., after treatment with a therapy, e.g., a complete response or partial response) and then progressed. In an embodiment, a therapeutic agent delivered by an erythroid cell described herein is more efficacious than the same therapeutic agent delivered freely, that is, not by an erythroid cell described herein, and is useful, e.g., to treat a tumor that has relapsed after treatment with that therapeutic agent. In an embodiment, a therapeutic agent delivered by an erythroid cell described herein is administered to treat a tumor that is has relapsed after treatment with a different therapeutic agent. In an embodiment, treatment with the erythroid cell is initiated during remission. In an embodiment, treatment with the erythroid cell is initiated after relapse.

A “tumor” as used herein refers to a plurality of aberrant cells having uncontrolled growth. The terms “tumor” and “cancer” are used interchangeably herein, e.g., both terms encompass solid and liquid, e.g., diffuse or circulating, tumors. In an embodiment the cancer or tumor is premalignant. In an embodiment, the cancer or tumor is malignant. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The terms “tumor cell” and “cancer cell” are used herein interchangeably to refer to a cell or a tumor or cancer.

“Tumor antigen” which is used herein interchangeably with “cancer antigen”, refers to a moiety (e.g., a peptide or protein) comprised by a tumor cell, e.g., present on the surface of the tumor. In embodiments, the tumor antigen is also present on one or more types of noncancerous cells (e.g., noncancerous cells in the subject having the cancer), e.g., at the same level or at a different level. In an embodiment the tumor antigen is present in higher average numbers on a tumor cell than on a non-tumor cell. In embodiments, an antigen is a polypeptide, or portion thereof, present on the tumor cell. An antigen may be bound by a binding partner, e.g., an antibody or a non-antibody binding partner.

As used herein, the term “variant” of a polypeptide refers to a polypeptide having at least one sequence difference compared to that polypeptide, e.g., one or more substitutions, insertions, or deletions. In some embodiments, the variant has at least 70%, 80%, 85%, 90%, 95%, 96%, 97%, 98%, or 99% identity to that polypeptide. A variant includes a fragment. In some embodiments, a fragment lacks up to 1, 2, 3, 4, 5, 10, 20, or 100 amino acids on the N-terminus, C-terminus, or both (each independently), compared to the full-length polypeptide.

As used herein with reference to a tumor, “vascularized” refers to a tumor that has induced angiogenesis and/or comprises tumor blood vessels. Tumor blood vessels differ from normal blood vessels and can be distinguished by histology, e.g., by an irregular shape and dilation; and may comprise tumor cells and endothelial cells.

### Exogenous Proteins

One or more of the exogenous proteins may have post-translational modifications characteristic of eukaryotic cells, e.g., mammalian cells, e.g., human cells. In some embodiments, one or more of the exogenous proteins are glycosylated, phosphorylated, or both. In vitro detection of glycoproteins is routinely accomplished on SDS-PAGE gels and Western Blots using a modification of Periodic acid-Schiff (PAS) methods. Cellular localization of glycoproteins may be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments, an exogenous polypeptide described herein is at least 200, 300, 400, 500, 600, 700, or 800 amino acids in length. In some embodiments, the exogenous polypeptide is between 200-300, 300-400, 400-500, 500-600, 600-700, or 700-800 amino acids in length. In some embodiments, the exogenous polypeptide is less than 500, 450, 400, 350, or 300 amino acids in length.

In embodiments, an erythroid cell described herein comprises at least 1,000, 5,000, 10,000, 15,000, 20,000, 25,000, 30,000, 50,000, 100,000, 200,000, or 500,000 copies of an exogenous polypeptide described herein. In embodiments, an erythroid cell described herein comprises about 200,000, 150,000-250,000, 100,000-300,000, or 200,000-300,000 copies of an exogenous polypeptide described herein, e.g., an exogenous polypeptide comprising 4-1BBL.

In embodiments, an exogenous polypeptide described herein forms a multimer, e.g., a dimer, trimer, tetramer, or hexamer. In embodiments, the exogenous polypeptide (e.g., comprising 4-1BBL) forms a trimer, e.g., a trimer at the surface of the erythroid cell.

In some embodiments, the erythroid cell comprises an agent, e.g., an exogenous polypeptide, e.g., a surface-exposed exogenous polypeptide, e.g., a surface-exposed polypeptide comprising a transmembrane domain, that binds a tumor antigen described herein, e.g., a tumor antigen of Table 1. In embodiments, the erythroid cell comprises an agent, e.g., an exogenous polypeptide, that comprises an antibody or other binding agent of Table 1 or Table 2, or a fragment or variant thereof. For instance, the fragment or variant could be a single domain antibody, e.g., scFv, corresponding to an antibody of Table 1 or Table 2. As another example, the fragment or variant could be an antigen-binding fragment of an antibody of Table 1 or Table 2, e.g., a light chain variable fragment, heavy chain variable fragment, or both. As another example, the fragment or variant could be an active fragment of a binding agent of Table 1 or Table 2. As another example, the fragment or variant could be an antibody having less than 100% sequence identity to an antibody of Table 1 or Table 2, e.g., an antibody having at least 70%, 75%, 80%, 95%, 96%, 97%, 98%, 99%, or 99.5% identity to the antibody of Table 1 or Table 2 or to a light chain variable fragment, heavy chain variable fragment, or both. For instance, the fragment or variant could have the same CDRs as an antibody of Table 1 or Table 2, but one or more mutations in the framework and/or constant region. As another example, the fragment or variant could be a binding agent having less than 100% sequence identity to a binding agent of Table 1 or Table 2, e.g., a binding agent having at least 70%, 75%, 80%, 95%, 96%, 97%, 98%, 99%, or 99.5% identity to a binding agent of Table 1 or Table 2 or to an active portion thereof.

In some embodiments, one or more of the exogenous polypeptides is a fusion protein, e.g., is a fusion with an endogenous red blood cell protein or fragment thereof. In embodiments, the exogenous polypeptide comprises a transmembrane protein, e.g., a Type I, Type II, or Type III red blood cell transmembrane protein or transmembrane fragment thereof. In embodiments, the transmembrane protein is GPA or a transmembrane fragment thereof.

In embodiments, the transmembrane domain comprises GPA or a transmembrane portion thereof, e.g., as set out in SEQ ID NO: 1 herein or a transmembrane portion thereof, or a polypeptide having at least 70%, 75%, 80%, 85%, 90%, 95%, 96%, 97%, 98%, 99% identity to any of the foregoing. In embodiments, the GPA polypeptide is C-terminal of the binding domain. In embodiments, the GPA polypeptide has a sequence of:

(SEQ ID NO: 1)
LSTTEVAMHTSTSSSVTKSYISSQTNDTHKRDTYAATPRAHEVSEISVRT
VYPPEEETGERVQLAHHFSEPEITLIIFGVMAGVIGTILLISYGIRRLIK
KSPSDVKPLPSPDTDVPLSSVEIENPETSDQ

or a transmembrane portion thereof, or a polypeptide having at least 70%, 75%, 80%, 85%, 90%, 95%, 96%, 97%, 98%, 99% identity to any of the foregoing. In embodiments, the GPA polypeptide is C-terminal of the antibody domain.

In embodiments, the exogenous polypeptide comprises a polypeptide of SEQ ID NO: 80 or SEQ ID NO: 81, or an amino acid sequence having at least 70%, 75%, 80%, 85%, 90%, 95%, 96%, 97%, 98%, 99%, or 99.5% identity thereto, or a functional fragment thereof. In embodiments, the functional fragment of 4-1BBL is a 4-1BB binding fragment. In embodiments, the functional fragment of anti-PD-L1 is a PD-L1 binding fragment.

In an embodiment, the exogenous polypeptide comprises a ligand for a cellular receptor that mediates apoptosis. In an embodiment the ligand is a TRAIL receptor ligand, e.g., wild-type or mutant TRAIL polypeptides, or antibody that binds TRAIL receptors, and induces apoptosis in a target cell. In some embodiments, an enucleated erythroid cell comprises one or more (e.g., two or three) TRAIL receptor ligands. In some embodiments, enucleated erythroid cell comprising one or more TRAIL receptor ligands further comprises a targeting moiety, e.g., a targeting moiety described herein.

TRAIL (TNF-related apoptosis inducing ligand) is a member of the TNF family that induces apoptosis. TRAIL has at least two receptors, TRAIL R1 and TRAIL R2. TRAIL receptor agonists, e.g., mutants of TRAIL that bind one or more of the receptors, or antibodies that bind one or both of TRAIL R1 or TRAIL R2 (see, e.g. Gasparian et al., Apoptosis 2009 Jun. 14(6), Buchsbaum et al. Future Oncol 2007 Aug. 3(4)), have been developed as a clinical therapy for a wide range of cancers. In embodiments, the enucleated erythroid cell comprises a TRAIL R1 ligand and a TRAIL R2 ligand.

In some embodiments, one or more of the exogenous polypeptides comprises a member of the TNF superfamily or a portion thereof. In some embodiments, the exogenous polypeptides bind to one or both of death receptors DR4 (TRAIL-R1) and DR5 (TRAIL-R2). In some embodiments, the exogenous polypeptides bind to one or more of TNFRSF10A/TRAILR1, TNFRSF10B/TRAILR2, TNFRSF10C/TRAILR3, TNFRSF10D/TRAILR4, or TNFRSF11B/OPG. In some embodiments, the exogenous polypeptides activate one or more of MAPK8/JNK, caspase 8, and caspase 3.

In some embodiments, a TRAIL polypeptide is a TRAIL agonist having a sequence of any of SEQ ID NOS: 2-6 herein, or a sequence with at least 70%, 80%, 85%, 90%, 95%, 96%, 97%, 98%, or 99% identity thereto. Sequence identity is measured, e.g., by BLAST (Basic Local Alignment Search Tool). SEQ ID Nos. 2-6 are further described in Mohr et al. BMC Cancer (2015) 15:494), which is herein incorporated by reference in its entirety.

Soluble TRAIL variant DR4-1
SEQ ID NO: 2
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRRRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG
Soluble TRAIL variant DR4-2
SEQ ID NO: 3
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRRGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG
Soluble TRAIL variant DR4-3
SEQ ID NO: 4
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRRRSNTLSSPNSKNEKALGIK
INSWESSRRGHSFLSNLHLRNGELVIHEKGFYYIYSQTYFRFQEEIKENT
KNDKQMVQYIYKYTDYPDPILLMKSARNSCWSKDAEYGLYSIYQGGIFEL
KENDRIFVSVTNEHLIDMDHEASFFGAFLVG
Soluble TRAIL variant DR5-1
SEQ ID NO: 5
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMHHEASFFGAFLVG
Soluble TRAIL variant DR5-2
SEQ ID NO: 6
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQERIKENT
KNDKQMVQYIYKYTSYPDPILLMKSARNSCWSKDAEYGLYSIYQGGIFEL
KENDRIFVSVTNEHLIDMHHEASFFGAFLVG

In some embodiments, the TRAIL receptor ligand comprises an antibody, e.g., an antibody fragment. In embodiments, the antibody recognizes one or both of TRAIL R1 and TRAIL R2. The antibody may be, e.g., Mapatumumab (human anti-DR4 mAb), Tigatuzumab (humanized anti-DR5 mAb), Lexatumumab (human anti-DR5 mAb), Conatumumab (human anti-DR5 mAb), or Apomab (human anti-DR5 mAb). In some embodiments, erythroid cell comprises at least one antibody that binds a TRAIL receptor and at least one TRAIL polypeptide.

In some embodiments, the erythroid cell comprises a binding agent with targeting activity. In embodiments, the binding agent also has anti-cancer activity, e.g., the same exogenous polypeptide is an anti-cancer agent and a targeting agent. In embodiments, the erythroid cell comprises a second agent, e.g., a second exogenous polypeptide, having anti-cancer activity. In embodiments, the targeting agent binds at or near a cancer cell, e.g., solid tumor cell, and the second agent (e.g., second polypeptide) has an anti-cancer function. In some embodiments the site of action is tumor vasculature. In embodiments, the targeting agent binds a marker of neovasculature, e.g. binds an integrin such as avB1, avB3, or avB5, or a4b1 integrins, e.g. a synthetic peptide knottin (Kim et al, JACS 2016, 137(1)) or an endogenous or natural ligand, e.g. echistatin, RGD, EETI2.5F, or VCAM-1, or binds prostate-specific membrane antigen, which is also found abundantly on neovasculature. In some embodiments, the targeting agent binds a cancer cell marker such as CD269 (expressed, e.g., in multiple myeloma cells) or CD123 (expressed, e.g., in ALM cells), CD28 (expressed, e.g., in T cells; CD28 can be bound by CD80/CD86), NY-ESO-1 (expressed, e.g., in ovarian cancer).

In embodiments, the anti-cancer agent comprises an enzyme, e.g., asparaginase, methionine gamma lyase (MGL), serine dehydrogenase, or fragment or variant thereof, that degrades metabolites that are selectively required by tumor cells to grow. The anti-cancer agent may be an inhibitor of angiogenesis, e.g. an inhibitor of angiopoietin or an inhibitor of VEGF or VEGFR to prevent further growth of blood vessels. The anti-cancer agent may be an immunostimulatory molecule to activate T cells, e.g., a costimulatory molecule (e.g., a costimulatory molecule of Table 5), an immune checkpoint inhibitor (see, e.g., Table 4) or a cytokine or a T cell activation ligand. The anti-cancer agent may bind an immune effector cell, e.g. a T cell or an inflammatory macrophage and may capture and bring the effector cell into proximity of the tumor. The anti-cancer agent may be a direct mediator of cell killing, e.g. TRAIL or FAS-L or other death ligands, or a toxin. In some embodiments, the anti-cancer agent comprises an agonist of a TRAIL receptor, e.g., an agonistic antibody. In embodiments, the anti-cancer agent is a pro-apoptotic agent. In embodiments, the anti-cancer agent comprises an adjuvant. For any of these anti-cancer agents, the net result is a red cell therapeutic (RCT) that localizes to a tumor site and thus concentrates its anti-tumor effect in a location that increases its efficacy.

In some embodiments, the exogenous polypeptide inhibits an immune checkpoint molecule. In embodiments, the exogenous polypeptide is situated at the surface of the erythroid cell (e.g., comprises a transmembrane portion and a surface-exposed portion) and binds an immune checkpoint molecule.

In some embodiments, the exogenous polypeptide inhibits an immune checkpoint molecule. In one embodiment, the inhibitor of the immune checkpoint molecule is an inhibitory antibody (e.g., an antibody such as a monospecific antibody, monoclonal antibody, or a single chain antibody). The antibody may be, e.g., humanized or fully human. In other embodiments, the inhibitor of the immune checkpoint molecule is a fusion protein, e.g., an Fc-receptor fusion protein. In some embodiments, the inhibitor of the immune checkpoint molecule is an agent, such as an antibody, that interacts with an immune checkpoint protein. In some embodiments, the inhibitor of the immune checkpoint molecule is an agent, such as an antibody, that interacts with the ligand of an immune checkpoint receptor. In one embodiment, the inhibitor of the immune checkpoint molecule is an inhibitor (e.g., an inhibitory antibody or small molecule inhibitor) of CTLA-4 (e.g., an anti-CTLA4 antibody such as ipilimumab/Yervoy or tremelimumab). In one embodiment, the inhibitor of the immune checkpoint molecule is an inhibitor (e.g., an inhibitory antibody or small molecule inhibitor) of PD-1 (e.g., nivolumab/Opdivo®; pembrolizumab/Keytruda®; pidilizumab/CT-011). In one embodiment, the inhibitor of the immune checkpoint molecule is an inhibitor (e.g., an inhibitory antibody or small molecule inhibitor) of PD-L1 (e.g., MPDL3280A/RG7446; MEDI4736; MSB0010718C; BMS 936559). In one embodiment, the inhibitor of the immune checkpoint molecule is an inhibitor (e.g., an inhibitory antibody or Fc fusion or small molecule inhibitor) of PDL2 (e.g., a PDL2/Ig fusion protein such as AMP 224). In one embodiment, the inhibitor of the immune checkpoint molecule is an inhibitor (e.g., an inhibitory antibody or small molecule inhibitor) of B7-H3 (e.g., MGA271), B7-H4, BTLA, HVEM, TIM3, GALS, LAGS, VISTA, KIR, 2B4, CD160, CGEN-15049, CHK 1, CHK2, A2aR, B-7 family ligands, or a combination thereof.

Inhibitors of immune checkpoint molecules can be broken down into at least 4 major categories: i) agents such as antibody that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, or KIR), ii) agents such as antibodies that activate stimulatory pathways directly on T cells or NK cells (e.g., antibodies targeting OX40, GITR, or 4-1BB), iii) agents such as antibody that block a suppressive pathway on immune cells or rely on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, or Ly6G), and iv) agents such as antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, or MUC1). Such agents described herein can be designed and produced, e.g., as described in Templeton, Gene and Cell Therapy, 2015; Green and Sambrook, Molecular Cloning, 2012.

In some embodiments, the erythroid cell comprises an agent, e.g., an exogenous polypeptide, e.g., a surface-exposed exogenous polypeptide, e.g., a surface-exposed polypeptide comprising a transmembrane domain, that binds an immune checkpoint molecule of Table 4. In embodiments, the erythroid cell comprises an agent, e.g., an exogenous polypeptide, that comprises an antibody or other binding agent of Table 4, or a fragment or variant thereof. For instance, the fragment or variant could be a single domain antibody, e.g., scFv, corresponding to an antibody of Table 4. As another example, the fragment or variant could be an antigen-binding fragment of an antibody of Table 4, e.g., a light chain variable fragment, heavy chain variable fragment, or both. As another example, the fragment or variant could be an active fragment of a binding agent of Table 4. As another example, the fragment or variant could be an antibody having less than 100% sequence identity to an antibody of Table 4, e.g., an antibody having at least 70%, 75%, 80%, 95%, 96%, 97%, 98%, 99%, or 99.5% identity to the antibody of Table 4 or to a light chain variable fragment, heavy chain variable fragment, or both. For instance, the fragment or variant could have the same CDRs as an antibody of Table 4, but one or more mutations in the framework and/or constant region. As another example, the fragment or variant could be a binding agent having less than 100% sequence identity to a binding agent of Table 4, e.g., a binding agent having at least 70%, 75%, 80%, 95%, 96%, 97%, 98%, 99%, or 99.5% identity to a binding agent of Table 4 or to an active portion thereof.

In some embodiments, the erythroid cell comprises an agent, e.g., an exogenous polypeptide, e.g., a surface-exposed exogenous polypeptide, e.g., a surface-exposed polypeptide comprising a transmembrane domain, that comprises a costimulatory molecule of Table 5 or a fragment or variant thereof. In embodiments, the erythroid cell comprises an agent, e.g., an exogenous polypeptide, that comprises a costimulatory molecule of Table 5 or a fragment or variant thereof. For instance, the fragment or variant could be a protein sequence having less than 100% sequence identity to a costimulatory molecule of Table 5, e.g., a costimulatory molecule having at least 70%, 75%, 80%, 95%, 96%, 97%, 98%, 99%, or 99.5% identity to the costimulatory molecule. In embodiments, the fragment or variant is an isoform of the costimulatory molecule, e.g., isoform 1, 2, 3, 4, or 5 of a costimulatory molecule of Table 5, or a fragment or variant thereof. In embodiments, the fragment or variant is a mature form of a costimulatory molecule of Table 5, e.g., has a sequence of the processed form of the costimulatory molecule of Table 5.

In embodiments, a costimulatory molecule comprises one or more of a MHC class I molecule, BTLA, a Toll ligand receptor, OX40, CD27, CD28, CDS, ICAM-1, LFA-1 (CD11a/CD18), ICOS (CD278), and 4-1BB (CD137), CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a, or a fragment or variant thereof.

As another example, an erythroid cell brings an immune effector cell (e.g., T cell) and a cancer cell in close proximity with one another to facilitate the killing of the cancer cell by the immune effector cell. Thus, in some embodiments, the first polypeptide binds a cell surface marker of a cancer cell and the second polypeptide binds a cell surface marker of an immune effector cell. The first and second polypeptides may comprise, e.g., antibodies. In some embodiments, the cancer cell marker is selected from CD19 (expressed, e.g., in B cell acute leukemia), EpCAM (expressed, e.g., in CTCs), CD20 (expressed, e.g., in B cell acute leukemia), CD45 (expressed, e.g., in CTCs), EGFR, HER2 (expressed, e.g., in breast cancer cells). In some embodiments, the immune cell marker is CD3.

Vehicles for Polypeptides Described Herein

While in many embodiments herein, the one or more exogenous polypeptides are situated on or in an erythroid cell, it is understood that any exogenous polypeptide(s) described herein can also be situated on or in another vehicle. The vehicle can comprise, e.g., a cell, an erythroid cell, a corpuscle, a nanoparticle, a micelle, a liposome, or an exosome. The vehicle may be, e.g., a particle such as a nanoparticle or a particle greater than 100, 200, 300, 400, 500, 1,000, 1,500, 2,000, or 2,500 nm in diameter. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected from a polypeptide of any of Table 2, Table 4, or Table 5, or a fragment or variant thereof, or an agonist or antagonist thereof, or an antibody thereto. In some embodiments, the vehicle comprises two or more agents described herein, e.g., any pair of agents described herein.

In some embodiments, the vehicle comprises an erythroid cell. In embodiments, the erythroid cell is a nucleated red blood cell, red blood cell precursor, or enucleated red blood cell. In embodiments, the erythroid cell is a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, or a combination thereof. In some embodiments, the erythroid cells are immortal or immortalized cells. In some embodiments, the vehicle comprises an immune effector cell, e.g., a B cell, T cell, or NK cell. In some embodiments, the cell is autologous or allogeneic.

### Heterogeneous Populations of Cells

While in many embodiments herein, one or more (e.g., two or more) exogenous polypeptides are situated on or in a single cell, it is understood that any polypeptide or combination of polypeptides described herein can also be situated on a plurality of cells. For instance, in some aspects, the disclosure provides a plurality of erythroid cells, wherein a first cell of the plurality comprises a first exogenous polypeptide and a second cell of the plurality comprises a second exogenous polypeptide. In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In embodiments, the membrane comprises a polysaccharide, e.g., an anionic polysaccharide alginate. In embodiments, the semipermeable membrane does now allow cells to pass through, but allows passage of small molecules or macromolecules, e.g., metabolites, proteins, or DNA. In embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

### Physical Characteristics of Enucleated Erythroid Cells

In some embodiments, the enucleated erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the enucleated erythroid cell comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the enucleated erythroid cell exhibits substantially the same osmotic membrane fragility as an isolated, uncultured enucleated erythroid cell that does not comprise an exogenous polypeptide. In some embodiments, the population of enucleated erythroid cells has an osmotic fragility of less than 50% cell lysis at 0.3%, 0.35%, 0.4%, 0.45%, or 0.5% NaCl. Osmotic fragility is determined, in some embodiments, using the method of Example 59 of WO2015/073587.

Cell Size

In some embodiments, the enucleated erythroid cell has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cell is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cell is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cell is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587.

Phosphatidylserine Content

In some embodiments, the enucleated erythroid cell has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587.

Other Characteristics

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the enucleated erythroid cells have a half-life of at least 30, 45, or 90 days in a subject.

In some embodiments, a population of cells comprising erythroid cells comprises less than about 10, 5, 4, 3, 2, or 1% echinocytes.

In some embodiments, an erythroid cell is enucleated. In some embodiments, a cell, e.g., an erythroid cell, contains a nucleus that is non-functional, e.g., has been inactivated.

### Universal Donor Erythroid Cells

In some embodiments, erythroid cells described herein are autologous and/or allogeneic to the subject to which the cells will be administered. For example, erythroid cells allogeneic to the subject include one or more of blood type specific erythroid cells (e.g., the cells can be of the same blood type as the subject) or one or more universal donor erythroid cells. In some embodiments, the enucleated erythroid cells described herein have reduced immunogenicity compared to a reference cell, e.g., have lowered levels of one or more blood group antigens.

Where allogeneic cells are used for transfusion, a compatible ABO blood group can be chosen to prevent an acute intravascular hemolytic transfusion reaction. The ABO blood types are defined based on the presence or absence of the blood type antigens A and B, monosaccharide carbohydrate structures that are found at the termini of oligosaccharide chains associated with glycoproteins and glycolipids on the surface of the erythrocytes (reviewed in Liu et al., Nat. Biotech. 25:454-464 (2007)). Because group O erythrocytes contain neither A nor B antigens, they can be safely transfused into recipients of any ABO blood group, e.g., group A, B, AB, or O recipients. Group O erythrocytes are considered universal and may be used in all blood transfusions. Thus, in some embodiments, an erythroid cell described herein is type O. In contrast, group A erythroid cells may be given to group A and AB recipients, group B erythroid cells may be given to group B and AB recipients, and group AB erythroid cells may be given to AB recipients.

In some instances, it may be beneficial to convert a non-group O erythroid cell to a universal blood type. Enzymatic removal of the immunodominant monosaccharides on the surface of group A and group B erythrocytes may be used to generate a population of group O-like erythroid cells (See, e.g., Liu et al., Nat. Biotech. 25:454-464 (2007)). Group B erythroid cells may be converted using an α-galactosidase derived from green coffee beans. Alternatively or in addition, α-N-acetylgalactosaminidase and α-galactosidase enzymatic activities derived from E. meningosepticum bacteria may be used to respectively remove the immunodominant A and B antigens (Liu et al., Nat. Biotech. 25:454-464 (2007)), if present on the erythroid cells. In one example, packed erythroid cells isolated as described herein, are incubated in 200 mM glycine (pH 6.8) and 3 mM NaCl in the presence of either α-N-acetylgalactosaminidase and α-galactosidase (about 300m/ml packed erythroid cells) for 60 min at 26° C. After treatment, the erythroid cells are washed by 3-4 rinses in saline with centrifugation and ABO-typed according to standard blood banking techniques.

While the ABO blood group system is the most important in transfusion and transplantation, in some embodiments it can be useful to match other blood groups between the erythroid cells to be administered and the recipient, or to select or make erythroid cells that are universal for one or more other (e.g., minor) blood groups. A second blood group is the Rh system, wherein an individual can be Rh+ or Rh−. Thus, in some embodiments, an erythroid cell described herein is Rh−. In some embodiments, the erythroid cell is Type O and Rh−.

In some embodiments, an erythroid cell described herein is negative for one or more minor blood group antigens, e.g., Le(a-b-) (for Lewis antigen system), Fy(a-b-) (for Duffy system), Jk(a-b-) (for Kidd system), M-N- (for MNS system), K-k- (for Kell system), Lu(a-b-) (for Lutheran system), and H-antigen negative (Bombay phenotype), or any combination thereof. In some embodiments, the erythroid cell is also Type O and/or Rh−. Minor blood groups are described, e.g., in Agarwal et al “Blood group phenotype frequencies in blood donors from a tertiary care hospital in north India” Blood Res. 2013 March; 48(1): 51-54 and Mitra et al “Blood groups systems” Indian J Anaesth. 2014 September-October; 58(5): 524-528, each of which is incorporated herein by reference in its entirety.

### Methods of Manufacturing Enucleated Erythroid Cells

Methods of manufacturing enucleated erythroid cells comprising an exogenous polypeptide are described, e.g., in WO2015/073587 and WO2015/153102, each of which is incorporated by reference in its entirety.

In some embodiments, hematopoietic progenitor cells, e.g., CD34+ hematopoietic progenitor cells, are contacted with a nucleic acid or nucleic acids encoding one or more exogenous polypeptides, and the cells are allowed to expand and differentiate in culture.

The nucleic acid may be, e.g., DNA or RNA. A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the cells are produced using conjugation, e.g., sortagging, e.g., as described in WO2014/183071 or WO2014/183066, each of which is incorporated by reference in its entirety.

In some embodiments, the erythroid cells are expanded at least 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated erythroid cells. In some embodiments, the population of erythroid cells contains less than 1% live enucleated cells, e.g., contains no detectable live enucleated cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by FACS using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

Methods of Treatment with Compositions Herein, e.g., Erythroid Cells

Methods of administering erythroid cells comprising (e.g., expressing) exogenous agent (e.g., polypeptides) are described, e.g., in WO2015/073587 and WO2015/153102, each of which is incorporated by reference in its entirety.

In embodiments, the erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In some embodiments, the erythroid cells are administered to a patient every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some aspects, the present disclosure provides a method of treating a disease or condition described herein, comprising administering to a subject in need thereof a composition described herein, e.g., an erythroid cell described herein. In some embodiments, the disease or condition is a cancer, e.g., a cancer described herein. In some aspects, the disclosure provides a use of an erythroid cell described herein for treating a disease or condition described herein, e.g., a cancer. In some aspects, the disclosure provides a use of an erythroid cell described herein for manufacture of a medicament for treating a disease or condition described herein, e.g., a cancer.

Types of cancer include acute lymphoblastic leukaemia (ALL), acute myeloid leukaemia (AML), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukaemia (CLL), chronic myeloid leukaemia (CML), colon cancer, colorectal cancer, endometrial cancer, eye cancer, gallbladder cancer, gastric cancer, gestational trophoblastic tumors (GTT), hairy cell leukaemia, head and neck cancer, Hodgkin lymphoma, kidney cancer, laryngeal cancer, leukaemia, liver cancer, lung cancer, lymphoma, B-cell lymphoma, melanoma skin cancer, mesothelioma, men's cancer, molar pregnancy, mouth and oropharyngeal cancer, myeloma, nasal and sinus cancers, nasopharyngeal cancer, non-Hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non-melanoma), soft tissue sarcoma, stomach cancer, testicular cancer, thyroid cancer, unknown primary cancer, uterine cancer, vaginal cancer, and vulval cancer.

In some embodiments, the cancer is a highly immunogenic cancer, e.g., the cancer has (e.g., as determined by analysis of a cancer biopsy) one or more of the following characteristics: (a) tumor infiltrating lymphocytes (TIL), e.g., at least 1, 5, 10, 100 TIL per 1000 tumor cells; (b) mutations, e.g., 0.1 or more somatic mutations per megabase of tumor genomic DNA; (c) neoantigens, e.g., 1 or more neoantigen with one or more endogenous T cell receptor and/or one or more idiotype clone that recognizes a processed and presented moiety of the neoantigen; (d) tertiary lymphoid structures; (e) high expression of inflammatory gene expression, e.g., 2-fold increased expression of cytokines above baseline expression in non-cancerous tissue; and (f) immune cells exhibiting immunosuppressive phenotype, e.g. dendritic cells lacking cytokine expression. Methods of assessing these characteristics of the cancer are known (see, e.g., Clin Cancer Res. 2000 May; 6(5):1875-81; Nature. 2013 Aug. 22; 500(7463):415-21. doi: 10.1038/nature12477. Epub 2013 Aug. 14; Nature. 2014 Nov. 27; 515(7528):577-81. doi: 10.1038/nature13988; Trends Immunol. 2014 November; 35(11):571-80. doi: 10.1016/j.it.2014.09.006. Epub 2014 Oct. 22; Front Immunol. 2013 Dec. 11; 4:438. doi: 10.3389/fimmu.2013.00438; Eur J Cancer. 2009 January; 45(2):228-47. doi: 10.1016/j.ejca.2008.10.026.

In some embodiments, the tumor cell is in a primary tumor. In some embodiments, the tumor cell is other than a circulating tumor cell.

In some embodiments, the tumor comprises a mutation in a gene of Table 8. In some embodiments, the mutation in a gene of Table 8 is a mutation specified in the third column of Table 8. In embodiments, the cancer comprises at least two different mutations, e.g., a mutation in a gene in Table 8 (e.g., a mutation specified in the third column of Table 8) and a second mutation. In embodiments, the second mutation is a mutation in a gene in Table 8, e.g., a mutation specified in the third column of Table 8. In embodiments, some cells in the tumor comprise the mutation and other cells do not.

In some embodiments, the tumor does not comprise p53-null cells. In embodiments, the patient having the tumor is not heterozygous for a p53-null mutation.

The tumor mutation status can be determined by a number of methods, such as PCR, microarray, or nucleic acid sequencing, e.g., high throughput DNA sequencing.

In some embodiments, the tumor comprises a tumor antigen chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the tumor is resistant to, e.g., has been treated with and does not respond to, a therapy comprising an antibody of Table 1 or Table 2, or a fragment or variant thereof. For instance, the fragment or variant could be a single domain antibody, e.g., scFv, corresponding to an antibody of Table 1 or Table 2. As another example, the fragment or variant could be an antigen-binding fragment of an antibody of Table 1 or Table 2, e.g., a light chain variable fragment, heavy chain variable fragment, or both. As another example, the fragment or variant could be an active fragment of a binding agent of Table 1 or Table 2. As another example, the fragment or variant could be an antibody having less than 100% sequence identity to an antibody of Table 1 or Table 2, e.g., an antibody having at least 70%, 75%, 80%, 95%, 96%, 97%, 98%, 99%, or 99.5% identity to the antibody of Table 1 or Table 2 or to a light chain variable fragment, heavy chain variable fragment, or both. For instance, the fragment or variant could have the same CDRs as an antibody of Table 1 or Table 2, but one or more mutations in the framework and/or constant region. As another example, the fragment or variant could be a binding agent having less than 100% sequence identity to a binding agent of Table 1 or Table 2, e.g., a binding agent having at least 70%, 75%, 80%, 95%, 96%, 97%, 98%, 99%, or 99.5% identity to a binding agent of Table 1 or Table 2 or to an active portion thereof.

In some embodiments, the erythroid cell comprises a chemotherapeutic agent. In some embodiments, the erythroid cell is administered in combination with a chemotherapeutic agent, e.g., the erythroid cell is administered before, after, or simultaneously with the chemotherapeutic agent. In some embodiments, the erythroid cell and the chemotherapeutic agent are admixed, and in some embodiments, they are in separate preparations. The erythroid cell and the chemotherapeutic agent may be administered through the same route of administration (e.g., intravenous) or different routes of administration (e.g., intravenous and oral).

In embodiments, the chemotherapeutic is a microtubule inhibitor, DNA replication inhibitor, photosensitizer, or enzyme inhibitor. In embodiments, the chemotherapeutic is a microtubule inhibitor (e.g., blocks microtubule assembly for instance a vinca alkaloid, or blocks microtubule disassembly such as a taxane or epothilone). In embodiments, the chemotherapeutic is a DNA replication inhibitor (e.g., an antimetabolite such as an antimetabolite of folic acid, a purine, a pyrimidine, or DNA; a topoisomerase inhibitor such as an inhibitor of Topoisomerase I or II; or a DNA crosslinker such as an alkylating agent, platinum-based agent, nonclassical DNA crosslinker, or intercalator). In embodiments, the photosensitizer is a porphyrin derivative.

More specifically, in some embodiments, the vinca alkaloid is chosen from vinblastine, Vincristine, Vinflunine, Vindesine, or Vinorelbine. In some embodiments, the taxane is chosen from Cabazitaxel, Docetaxel, Larotaxel, Ortataxel, Paclitaxel, or Tesetaxel. In some embodiments, the folic acid antimetabolite is selected from a Dihydrofolate reductase inhibitor (e.g., Aminopterin, Methotrexate, Pemetrexed, or Pralatrexate) or a Thymidylate synthase inhibitor (e.g., Raltitrexed or Pemetrexed). In some embodiments, the purine antimetabolite is selected from an Adenosine deaminase inhibitor (e.g., Pentostatin), a halogenated/ribonucleotide reductase inhibitor (e.g., Cladribine, Clofarabine, Fludarabine), or a Thiopurine (e.g., Thioguanine or Mercaptopurine). In some embodiments, the pyrimidine antimetabolite is chosen from a Thymidylate synthase inhibitor (e.g., Fluorouracil, Capecitabine, Tegafur, Carmofur, or Floxuridine), a DNA polymerase inhibitor (e.g., Cytarabine), a Ribonucleotide reductase inhibitor (e.g., Gemcitabine) or a Hypomethylating agent (e.g., Azacitidine of Decitabine). In some embodiments, the Deoxyribonucleotide antimetabolite is a Ribonucleotide reductase inhibitor (e.g., Hydroxycarbamide). In embodiments, the topoisomerase inhibitor is an inhibitor of topoisomerase I, e.g., a Camptotheca compound, e.g., Camptothecin, Cositecan, Belotecan, Gimatecan, Exatecan, Irinotecan, Lurtotecan, Silatecan, Topotecan, or Rubitecan. In embodiments, the topoisomerase inhibitor is an inhibitor of topoisomerase II, e.g., a Podophyllum compound (e.g., Etoposide or Teniposide). In embodiments, the topoisomerase inhibitor has intercalation activity, e.g., an Anthracycline (e.g., Aclarubicin, Daunorubicin, Doxorubicin, Epirubicin, Idarubicin, Amrubicin, Pirarubicin, Valrubici, orn Zorubicin) or an Anthracenedione (e.g., Mitoxantrone or Pixantrone).

In embodiments, the alkylating agent is a Nitrogen mustard, e.g., Mechlorethamine, a Cyclophosphamide (e.g., Ifosfamide or Trofosfamide), a Chlorambucil (e.g., Melphalan or Prednimustine), Bendamustine, Uramustine, or Estramustine. In embodiments, the alkylating agent is a Nitrosourea, e.g., Carmustine, Lomustine, Fotemustine, Nimustine, Ranimustine, or Streptozocin. In embodiments, the alkylating agent is an Alkyl sulfonate, e.g., a Busulfan (e.g., Mannosulfan or Treosulfan). In embodiments, the alkylating agent is an Aziridine, e.g., Carboquone, ThioTEPA, Triaziquone, or Triethylenemelamine. In embodiments, the platinum-based agent is chosen from Carboplatin, Cisplatin, Dicycloplatin, Nedaplatin, Oxaliplatin, or Satraplatin. In embodiments, the nonclassical DNA crosslinker is a Hydrazine (e.g., Procarbazine), a Triazene (e.g., Dacarbazine or Temozolomide), Altretamine, Mitobronitol, or Pipobroman. In embodiments, the intercalator is a Streptomyces compound, e.g., (Actinomycin, Bleomycin, Mitomycin, or Plicamycin).

In embodiments, the photosensitizer is selected from an Aminolevulinic acid/Methyl aminolevulinate, Efaproxiral, a porphyrin derivative (e.g., Porfimer sodium Talaporfin, Temoporfin, or Verteporfin).

In embodiments, the enzyme inhibitor is selected from a Farnesyltransferase inhibitor (e.g., Tipifarnib), a CDK inhibitor (e.g., Alvocidib, Seliciclib, or Palbociclib), a Proteasome inhibitor (e.g., (Bortezomib, Carfilzomib, or Ixazomib), a Phosphodiesterase inhibitor (e.g., Anagrelide), an IMP dehydrogenase inhibitor (e.g., Tiazofurin), a Arachidonate 5-lipoxygenase inhibitor (e.g., Masoprocol), a PARP inhibitor (e.g., Olaparib or Rucaparib), an HDAC inhibitor (e.g., Belinostat, Panobinostat, Romidepsin, or Vorinostat) or a Phosphoinositide 3-kinase inhibitor, e.g., (Idelalisib).

In embodiments, the chemotherapeutic is a receptor antagonist. In embodiments, the receptor antagonist is chosen from an Endothelin receptor antagonist (e.g., Atrasentan), a Retinoid X receptor antagonist (e.g., Bexarotene), or a Sex steroid (e.g., Testolactone). In embodiments, the chemotherapeutic is selected from Amsacrine, Trabectedin, a Retinoid (e.g., Alitretinoin or Tretinoin), Arsenic trioxide, an Asparagine deplete (e.g., Asparaginase of Pegaspargase). Celecoxib, Demecolcine, Elesclomol, Elsamitrucin, Etoglucid, Lonidamine, Lucanthone, Mitoguazone, Mitotane, Oblimersen, Omacetaxine mepesuccinate, or Eribulin.

### Tables

TABLE 1
Therapeutic anti-cancer antibodies. This table comprises antibodies that
have received regulatory approval and/or have entered clinical trials.

| Antibody | Target | Exemplary indications |
|---|---|---|
| rituximab | CD20 | Lymphoma e.g., NHL, leukemia |
| alemtuzumab | CD52 | Leukemia, e.g., B cell leukemia |
| Trastuzumab, e.g., | HER2/neu | Solid tumors, e.g., breast cancer, e.g., HER2- |
| ado-trastuzumab, e.g., | | positive breast cancer, e.g., breast cancer with |
| ado-trastuzumab | | HER2 overexpression |
| emtansine | | |
| nimotuzumab | EGFR | Solid tumors, e.g., squamous cell carcinoma or glioma |
| cetuximab | EGFR | Solid tumors, e.g., squamous cell carcinoma or |
| | | colorectal carcinoma |
| bevacizumab | VEGF | Solid tumors, e.g., colon cancer, lung cancer, renal |
| | | cancer, ovarian cancer, glioma, breast cancer |
| bavituximab | phosphatidylserine | Solid tumors, e.g., lung cancer e.g. non-small cell |
| | | lung cancer, pancreatic cancer, hepatocellular |
| | | carcinoma, melanoma, rectal cancer, prostate |
| | | cancer |
| gemtuzumab, e.g., | CD33 | Leukemia, e.g., acute myelogenous leukemia |
| gemtuzumab | | |
| ozogamicin | | |
| ibritumomab, e.g., | CD20 | Lymphoma, e.g., NHL, e.g., B cell non-Hodgkin's |
| ibritumomab tiuxetan | | lymphoma |
| Tositumomab, e.g., | CD20 | Lymphoma, e.g., NHL, |
| tositumomab-I-131 | | |
| panitumumab | EGFR | Solid tumors, e.g., colorectal cancer |
| ofatumumab | CD20 | Leukemia, e.g., CLL, lymphoma e.g., NHL, e.g., |
| | | DLBCL |
| ipilimumab | CTLA-4 | Solid tumors, e.g., melanoma, bladder cancer, |
| | | prostate cancer, and lung cancer e.g. NSCLC and |
| | | SCLC |
| brentuximab, e.g., | CD30 | Lymphoma, e.g., Hodgkin lymphoma and sALCL |
| brentuximab vedotin | | |
| pertuzumab | Her2 | Solid tumors, e.g., breast cancer, e.g., HER2- |
| | | positive breast cancer, e.g., breast cancer with |
| | | HER2 overexpression |
| obinutuzumab | CD20 | Leukemia, e.g., CLL, and lymphoma, e.g., |
| | | follicular lymphoma |
| durvalumab | PD-L1 | Solid tumors, e.g., bladder cancer and lung cancer e.g., NSCLC |
| nivolumab | PD-1 | Solid tumors, e.g., renal cell carcinoma, melanoma |
| | | or lung cancer e.g., NSCLC |
| pembrolizumab | PD-1 | Solid tumors, e.g., HNSCC, melanoma, or lung |
| | | cancer e.g., NSCLC |
| olaratumab | PDGF-R α | Solid tumors, e.g., soft tissue sarcoma |
| atezolizumab | PD-L1 | Solid tumors, e.g., lung cancer, e.g., NSCLC |
| elotuzumab | SLAMF7 | Multiple myeloma |
| | (CD319) | |
| necitumumab | EGFR | Solid tumors, e.g., lung cancer, e.g., NSCLC |
| daratumumab | CD38 | Multiple myeloma |
| ramucirumab | VEGFR2 (KDR) | Solid tumors, e.g., colorectal cancer |
| dinutuximab | GD2 | Solid tumors, e.g., neuroblastoma |
| blinatumomab | CD19, CD3 | Leukemia, e.g., ALL |
| siltuximab | IL-6 | solid tumors, e.g., renal cell cancer, prostate |
| | | cancer |
| denosumab | RANK ligand | Solid tumors, e.g., giant cell tumor of bone |
| catumaxomab | EpCAM, CD3 | Solid tumors, e.g., head and neck cancer |
| enoblituzumab | B7H3 | Solid tumors, e.g., NSCLC, SCCHN, Melanoma, |
| | | Urothelial Cancer |
| tremelimumab | CTLA4 | Solid tumors, e.g., Melanoma, mesothelioma |
| lirilumab | KIR2D subgroup | Leukemia, e.g., AML, CLL; solid tumors |
| pidilizumab | PD1 | Lymphoma, e.g., DLBCL; multiple myeloma |
| avelumab | PDL1 | Solid tumors, e.g., Nasopharyngeal Cancer, |
| | | Endometrial Cancer, Merkel Cell Carcinoma, |
| | | Ovarian Cancer, Peritoneal Cancer, Fallopian |
| | | Tube Cancer; Leukemia e.g., AML |

TABLE 2
Antibody VH and VL sequences. The sequences in this table were
obtained from publically available sources and if there is a
discrepancy between a sequence herein and the sequence of the
named antibody, the actual antibody controls.

| Antibody | Sequence | |
|---|---|---|
| rituximab | >Rituximab heavy chain chimeric | |
| | QVQLQQPGAELVKPGASVKMSCKASGYTFTSYNMHWVKQTP | |
| | GRGLEWIGAIYPGNGDTSYNQKFKGKATLTADKSSSTAYMQLS | |
| | SLTSEDSAVYYCARSTYYGGDWYFNVWGAGTTVTVSAASTKG | |
| | PSVFPLAPSSKSTSGGTAALGCLVKDYFPEPVTVSWNSGALTSG | |
| | VHTFPAVLQSSGLYSLSSVVTVPSSSLGTQTYICNVNHKPSNTK | |
| | VDKKAEPKSCDKTHTCPPCPAPELLGGPSVFLFPPKPKDTLMISR | |
| | TPEVTCVVVDVSHEDPEVKFNWYVDGVEVHNAKTKPREEQYN | |
| | STYRVVSVLTVLHQDWLNGKEYKCKVSNKALPAPIEKTISKAK | |
| | GQPREPQVYTLPPSRDELTKNQVSLTCLVKGFYPSDIAVEWESN | |
| | GQPENNYKTTPPVLDSDGSFFLYSKLTVDKSRWQQGNVFSCSV | |
| | MHEALHNHYTQKSLSLSPGK (SEQ ID NO: 7) | |
| | >Rituximab light chain chimeric | |
| | QIVLSQSPAILSASPGEKVTMTCRASSSVSYIHWFQQKPGSSPKP | |
| | WIYATSNLASGVPVRFSGSGSGTSYSLTISRVEAEDAATYYCQQ | |
| | WTSNPPTFGGGTKLEIKRTVAAPSVFIFPPSDEQLKSGTASVVCL | |
| | LNNFYPREAKVQWKVDNALQSGNSQESVTEQDSKDSTYSLSST | |
| | LTLSKADYEKHKVYACEVTHQGLSSPVTKSFNRGEC | |
| | (SEQ ID NO: 8) | |
| alemtuzumab | >1CE1:H CAMPATH-1H:Heavy Chain 1 | |
| | QVQLQESGPGLVRPSQTLSLTCTVSGFTFTDFYMNWVRQPPGR | |
| | GLEWIGFIRDKAKGYTTEYNPSVKGRVTMLVDTSKNQFSLRLSS | |
| | VTAADTAVYYCAREGHTAAPFDYWGQGSLVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK (SEQ ID NO: 9) | |
| | >1CE1:L CAMPATH-1H:Light Chain 1 | |
| | DIQMTQSPSSLSASVGDRVTITCKASQNIDKYLNWYQQKPGKA | |
| | PKLLIYNTNNLQTGVPSRFSGSGSGTDFTFTISSLQPEDIATYYCL | |
| | QHISRPRTFGQGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNR | |
| | (SEQ ID NO: 10) | |
| Trastuzumab, e.g., | >Anti-HER2 Light chain (1 and 2) | |
| ado-trastuzumab, e.g., | DIQMTQSPSSLSASVGDRVTITCRASQDVNTAVAWYQQKPGKA | |
| ado-trastuzumab | PKLLIYSASFLYSGVPSRFSGSRSGTDFTLTISSLQPEDFATYYCQ | |
| emtansine | QHYTTPPTFGQGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | (SEQ ID NO: 11) | |
| | >Anti-HER2 Heavy chain (1 and 2) | |
| | EVQLVESGGGLVQPGGSLRLSCAASGFNIKDTYIHWVRQAPGK | |
| | GLEWVARIYPTNGYTRYADSVKGRFTISADTSKNTAYLQMNSL | |
| | RAEDTAVYYCSRWGGDGFYAMDYWGQGTLVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVD | |
| | KKVEPPKSCDKTHTCPPCPAPELLGGPSVFLFPPKPKDTLMISRT | |
| | PEVTCVVVDVSHEDPEVKFNWYVDGVEVHNAKTKPREEQYNS | |
| | TYRVVSVLTVLHQDWLNGKEYKCKVSNKALPAPIEKTISKAKG | |
| | QPREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGK (SEQ ID NO: 12) | |
| nimotuzumab | >pdb\|3gkw\|Heavy chain | |
| | QVQLQQSGAEVKKPGSSVKVSCKASGYTFTNYYIYWVRQAPG | |
| | QGLEWIGGINPTSGGSNFNEKFKTRVTITADESSTTAYMELSSLR | |
| | SEDTAFYFCTRQGLWFDSDGRGFDFWGQGTTVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVD | |
| | KKVP (SEQ ID NO: 13) | |
| | >pdb\|3gkw\|Light chain | |
| | DIQMTQSPSSLSASVGDRVTITCRSSQNIVHSNGNTYLDWYQQT | |
| | PGKAPKLLIYKVSNRFSGVPSRFSGSGSGTDFTFTISSLQPEDIAT | |
| | YYCFQYSHVPWTFGQGTKLQITREVAAPSVFIFPPSDEQLKSGT | |
| | ASVVCLLNNFYPREAKVQWKVDNALQSGNSQESVTEQDSKDS | |
| | TYSLSSTLTLSKADYEKHKVYACEVTHQGLSSPVTKSFNRGEC | |
| | (SEQ ID NO: 14) | |
| cetuximab | >Cetuximab heavy chain | |
| | QVQLKQSGPGLVQPSQSLSITCTVSGFSLTNYGVHWVRQSPGK | |
| | GLEWLGVIWSGGNTDYNTPFTSRLSINKDNSKSQVFFKMNSLQ | |
| | SNDTAIYYCARALTYYDYEFAYWGQGTLVTVSAASTKGPSVFP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKV | |
| | EPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 15) | |
| | >Cetuximab light chain | |
| | DILLTQSPVILSVSPGERVSFSCRASQSIGTNIHWYQQRTNGSPRL | |
| | LIKYASESISGIPSRFSGSGSGTDFTLSINSVESEDIADYYCQQNN | |
| | NWPTTFGAGTKLELKRTVAAPSVFIFPPSDEQLKSGTASVVCLL | |
| | NNFYPREAKVQWKVDNALQSGNSQESVTEQDSKDSTYSLSSTL | |
| | TLSKADYEKHKVYACEVTHQGLSSPVTKSFNRGEC | |
| | (SEQ ID NO: 16) | |
| bevacizumab | >“Bevacizumab light chain” | |
| | DIQMTQSPSSLSASVGDRVTITCSASQDISNYLNWYQQKPGKAP | |
| | KVLIYFTSSLHSGVPSRFSGSGSGTDFTLTISSLQPEDFATYYCQQ | |
| | YSTVPWTFGQGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | (SEQ ID NO: 17) | |
| | >“Bevacizumab heavy chain” | |
| | EVQLVESGGGLVQPGGSLRLSCAASGYTFTNYGMNWVRQAPG | |
| | KGLEWVGWINTYTGEPTYAADFKRRFTFSLDTSKSTAYLQMNS | |
| | LRAEDTAVYYCAKYPHYYGSSHWYFDVWGQGTLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPAPIEKTISKA | |
| | KGQPREPQVYTLPPSREEMTKNQVSLTCLVKGFYPSDIAVEWES | |
| | NGQPENNYKTTPPVLDSDGSFFLYSKLTVDKSRWQQGNVFSCS | |
| | VMHEALHNHYTQKSLSLSPGK (SEQ ID NO: 18) | |
| bavituximab | >8734_H\|bavituximab\|\|\|H-GAMMA-1 (VH(1-120) + CH1(121- | |
| | 218) + HINGE-REGION(219-233) + CH2(234-343) + CH3 | |
| | (344-450))\|\|\|\|\|\|\|450\|\|\|\|MW 49410.6\|MW 49410.6\| | |
| | EVQLQQSGPELEKPGASVKLSCKASGYSFTGYNMNWVKQSHG | |
| | KSLEWIGHIDPYYGDTSYNQKFRGKATLTVDKSSSTAYMQLKS | |
| | LTSEDSAVYYCVKGGYYGHWYFDVWGAGTTVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVD | |
| | KKVEPKSCDKTHTCPPCPAPELLGGPSVFLFPPKPKDTLMISRTP | |
| | EVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNGQ | |
| | PENNYKTTPPVLDSDGSFFLYSKLTVDKSRWQQGNVFSCSVMH | |
| | EALHNHYTQKSLSLSPGK (SEQ ID NO: 19) | |
| | >8734_L\|bavituximab\|\|\|L-KAPPA (V-KAPPA(1-107) + C-KAPPA | |
| | (108-208))\|\|\|\|\|\|\|208\|\|\|\|MW 22655.0\|MW 22655.0\| | |
| | DIQMTQSPSSLSASLGERVSLTCRASQDIGSSLNWLQQGPDGTIK | |
| | RLIYATSSLDSGVPKRFSGSRSGSDYSLTISSLESEDFVDYYCLQ | |
| | YVSSPPTFGAGTKLELKRADAAPSVFIFPPSDEQLKSGTASVVCL | |
| | LNNFYPREAKVQWKVDNALQSGNSQESVTEQDSKDSTYSLSK | |
| | ADYEKHKVYACEVTHQGLSSPVTKSFNRGEC | |
| | (SEQ ID NO: 20) | |
| gemtuzumab, e.g., | >Light Chain No. 1 | |
| gemtuzumab | QIVLTQSPAIMSASPGEKVTITCSASSSISYMHWFQQKPGTSPKL | |
| ozogamicin | WIYTTSNLASGVPARFSGSGSGTSYSLTISRMEAEDAATYYCHQ | |
| | RSTYPLTFGSGTKLELK (SEQ ID NO: 21) | |
| | >Light Chain No. 2 | |
| | DIQMTQSPSTLSASVGDRVTITCRASQSINTWLAWYQQKPGKAP | |
| | KLLMYKASSLESGVPSRFIGSGSGTEFTLTISSLQPDDFATYYCQ | |
| | QYNSDSKMFGQGTKVEVK (SEQ ID NO: 22) | |
| | >Heavy Chain No. 1 | |
| | QVQLQQSGAELAKPGASVKMSCKASGYTFTSYRMHWVKQRP | |
| | GQGLEWIGYINPSTGYTEYNQKFKDKATLTADKSSSTAYMQLS | |
| | SLTFEDSAVYYCARGGGVFDYWGQGTTLTVSS | |
| | (SEQ ID NO: 23) | |
| | >Heavy Chain No. 2 | |
| | QVQLVQSGAEVKKPGSSVKVSCKASGGTFSRSAIIWVRQAPGQ | |
| | GLEWMGGIVPMFGPPNYAQKFQGRVTITADESTNTAYMELSSL | |
| | RSEDTAFYFCAGGYGIYSPEEYNGGLVTVSS | |
| | (SEQ ID NO: 24) | |
| ibritumomab, e.g., | >Ibritumomab tiuxetan heavy chain | |
| ibritumomab tiuxetan | QAYLQQSGAELVRPGASVKMSCKASGYTFTSYNMHWVKQTPR | |
| | QGLEWIGAIYPGNGDTSYNQKFKGKATLTVDKSSSTAYMQLSS | |
| | LTSEDSAVYFCARVVYYSNSYWYFDVWGTGTTVTVSAPSVYP | |
| | LAPVCGDTTGSSVTLGCLVKGYFPEPVTLTWNSGSLSSGVHTFP | |
| | AVLQSDLYTLSSSVTVTSSTWPSQSITCNVAHPASSTKVDKKIEP | |
| | RGPTIKPCPPCKCPAPNLLGGPSVFIFPPKIKDVLMISLSPIVTCVV | |
| | VDVSEDDPDVQISWFVNNVEVHTAQTQTHREDYNSTLRVVSAL | |
| | PIQHQDWMSGKEFKCKVNNKDLPAPIERTISKPKGSVRAPQVY | |
| | VLPPPEEEMTKKQVTLTCMVTDFMPEDIYVEWTNNGKTELNY | |
| | KNTEPVLDSDGSYFMYSKLRVEKKNWVERNSYSCSVVHEGLH | |
| | NHHTTKSFSR (SEQ ID NO: 25) | |
| | >Ibritumomab tiuxetan light chain | |
| | QIVLSQSPAILSASPGEKVTMTCRASSSVSYMHWYQQKPGSSPK | |
| | PWIYAPSNLASGVPARFSGSGSGTSYSLTISRVEAEDAATYYCQ | |
| | QWSFNPPTFGAGTKLELKRADAAPTVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFN | |
| | (SEQ ID NO: 26) | |
| Tositumomab, e.g., | Mouse-Human chimeric Anti-CD20 Heavy Chain 1 | |
| tositumomab-I-131 | QAYLQQSGAELVRPGASVKMSCKASGYTFTSYNMHWVKQTPR | |
| | QGLEWIGAIYPGNGDTSYNQKFKGKATLTVDKSSSTAYMQLSS | |
| | LTSEDSAVYFCARVVYYSNSYWYFDVWGTGTTVTVSGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKAE | |
| | PKSCDKTHTCPPCPAPELLGGPSVFLFPPKPKDTLMISRTPEVTC | |
| | VVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 27) | |
| | >Mouse-Human chimeric Anti-CD20 Light Chain 1 | |
| | QIVLSQSPAILSASPGEKVTMTCRASSSVSYMHWYQQKPGSSPK | |
| | PWIYAPSNLASGVPARFSGSGSGTSYSLTISRVEAEDAATYYCQ | |
| | QWSFNPPTFGAGTKLELKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNR | |
| | (SEQ ID NO: 28) | |
| | >Mouse-Human chimeric Anti-CD20 Heavy Chain 2 | |
| | QAYLQQSGAELVRPGASVKMSCKASGYTFTSYNMHWVKQTPR | |
| | QGLEWIGAIYPGNGDTSYNQKFKGKATLTVDKSSSTAYMQLSS | |
| | LTSEDSAVYFCARVVYYSNSYWYFDVWGTGTTVTVSGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKAE | |
| | PKSCDKTHTCPPCPAPELLGGPSVFLFPPKPKDTLMISRTPEVTC | |
| | VVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 29) | |
| | >Mouse-Human chimeric Anti-CD20 Light Chain 2 | |
| | QIVLSQSPAILSASPGEKVTMTCRASSSVSYMHWYQQKPGSSPK | |
| | PWIYAPSNLASGVPARFSGSGSGTSYSLTISRVEAEDAATYYCQ | |
| | QWSFNPPTFGAGTKLELKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNR | |
| | (SEQ ID NO: 30) | |
| panitumumab | >pdb\|5sx5\|Heacy chain J | |
| | QVQLQESGPGLVKPSETLSLTCTVSGGSVSSGDYYWTWIRQSPG | |
| | KGLEWIGHIYYSGNTNYNPSLKSRLTISIDTSKTQFSLKLSSVTA | |
| | ADTAIYYCVRDRVTGAFDIWGQGTMVTVSSASTKGPSVFPLAP | |
| | CSRSTSESTAALGCLVKDYFPEPVTVSWNSGALTSGVHTFPAVL | |
| | QSSGLYSLSSVVTVPSSNFGTQTYTCNVDHKPSNTKVDKTVER | |
| | KC (SEQ ID NO: 31) | |
| | >pdb\|5sx5\|K Light chain | |
| | DIQMTQSPSSLSASVGDRVTITCQASQDISNYLNWYQQKPGKAP | |
| | KLLIYDASNLETGVPSRFSGSGSGTDFTFTISSLQPEDIATYFCQH | |
| | FDHLPLAFGGGTKVEIKRTVAAPSVFIFPPSDEQLKSGTASVVCL | |
| | LNNFYPREAKVQWKVDNALQSGNSQESVTEQDSKDSTYSLSST | |
| | LTLSKADYEKHKVYACEVTHQGLSSPVTKSFNRGEC | |
| | (SEQ ID NO: 32) | |
| ofatumumab | >Ofatumumab Heavy Chain | |
| | EVQLVESGGGLVQPGRSLRLSCAASGFTFNDYAMHWVRQAPG | |
| | KGLEWVSTISWNSGSIGYADSVKGRFTISRDNAKKSLYLQMNS | |
| | LRAEDTALYYCAKDIQYGNYYYGMDVWGQGTTVTVSSASTK | |
| | GPSVFPLAPGSSKSTSGTAALGCLVKDYFPEPVTVSWNSGALTS | |
| | GVHTFPAVLQSSGLYSLSSVVTVPSSSLGTQTYICNVNHKPSNT | |
| | KVDKKVEP (SEQ ID NO: 33) | |
| | >Ofatumumab Light Chain | |
| | EIVLTQSPATLSLSPGERATLSCRASQSVSSYLAWYQQKPGQAP | |
| | RLLIYDASNRATGIPARFSGSGSGTDFTLTISSLEPEDFAVYYCQ | |
| | QRSNWPITFGQGTRLEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNR | |
| | (SEQ ID NO: 34) | |
| ipilimumab | >Ipilimumab heavy chain | |
| | QVQLVESGGGVVQPGRSLRLSCAASGFTFSSYTMHWVRQAPG | |
| | KGLEWVTFISYDGNNKYYADSVKGRFTISRDNSKNTLYLQMNS | |
| | LRAEDTAIYYCARTGWLGPFDYWGQGTLVTVSSASTKGPSVFP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKRV | |
| | EPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 35) | |
| | >Ipilimumab light chain | |
| | EIVLTQSPGTLSLSPGERATLSCRASQSVGSSYLAWYQQKPGQA | |
| | PRLLIYGAFSRATGIPDRFSGSGSGTDFTLTISRLEPEDFAVYYCQ | |
| | QYGSSPWTFGQGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | (SEQ ID NO: 36) | |
| brentuximab, e.g., | >Brentuximab vedotin - heavy chain | |
| brentuximab vedotin | QIQLQQSGPEVVKPGASVKISCKASGYTFTDYYITWVKQKPGQ | |
| | GLEWIGWIYPGSGNTKYNEKFKGKATLTVDTSSSTAFMQLSSLT | |
| | SEDTAVYFCANYGNYWFAYWGQGTQVTVSAASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPG (SEQ ID NO: 37) | |
| | >Brentuximab vedotin - light chain | |
| | DIVLTQSPASLAVSLGQRATISCKASQSVDFDGDSYMNWYQQK | |
| | PGQPPKVLIYAASNLESGIPARFSGSGSGTDFTLNIHPVEEEDAA | |
| | TYYCQQSNEDPWTFGGGTKLEIKRTVAAPSVFIFPPSDEQLKSG | |
| | TASVVCLLNNFYPREAKVQWKVDNALQSGNSQESVTEQDSKD | |
| | STYSLSSTLTLSKADYEKHKVYACEVTHQGLSSPVTKSFNRGEC | |
| | (SEQ ID NO: 38) | |
| pertuzumab | >Amino acid sequence for pertuzumab light chain | |
| | DIQMTQSPSSLSASVGDRVTITCKASQDVSIGVAWYQQKPGKAP | |
| | KLLIYSASYRYTGVPSRFSGSGSGTDFTLTISSLQPEDFATYYCQ | |
| | QYYIYPYTFGQGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | (SEQ ID NO: 39) | |
| | >Amino acid sequence for pertuzumab heavy chain | |
| | EVQLVESGGGLVQPGGSLRLSCAASGFTFTDYTMDWVRQAPG | |
| | KGLEWVADVNPNSGGSIYNQRFKGRFTLSVDRSKNTLYLQMNS | |
| | LRAEDTAVYYCARNLGPSFYFDYWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKK | |
| | VEPKSCDKTHTCPPCPAPELLGGPSVFLFPPKPKDTLMISRTPEV | |
| | TCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG (SEQ ID NO: 40) | |
| obinutuzumab | >Amino acid sequence of the light chains of obinutuzumab | |
| | (SEQ ID NO: 41) | |
| | 1 DIVMTQTPLSLPVTPGEPASISCRSSKSLLHSNGITYLYWYLQKPGQSPQ | |
| | 51 LLIYQMSNLVSGVPDRFSGSGSGTDFTLKISRVEAEDVGVYYCAQNLELP | |
| | 101 YTFGGGTKVEIKRTVAAPSVFIFPPSDEQLKSGTASVVCLLNNFYPREAK | |
| | 151 VQWKVDNALQSGNSQESVTEQDSKDSTYSLSSTLTLSKADYEKHKVYACE | |
| | 201 VTHQGLSSPVTKSFNRGEC | 219 |
| | >Amino acid sequence of the heavy chains of obinutuzumab | |
| | (SEQ ID NO: 42) | |
| | 1 QVQLVQSGAEVKKPGSSVKVSCKASGYAFSYSWINWVRQAPGQGLEWMGR | |
| | 51 IFPGDGDTDYNGKFKGRVTITADKSTSTAYMELSSLRSEDTAVYYCARNV | |
| | 101 FDGYWLVYWGQGTLVTVSSASTKGPSVFPLAPSSKSTSGGTAALGCLVKD | |
| | 151 YFPEPVTVSWNSGALTSGVHTFPAVLQSSGLYSLSSVVTVPSSSLGTQTY | |
| | 201 ICNVNHKPSNTKVDKKVEPKSCDKTHTCPPCPAPELLGGPSVFLFPPKPK | |
| | 251 DTLMISRTPEVTCVVVDVSHEDPEVKFNWYVDGVEVHNAKTKPREEQYNS | |
| | 301 TYRVVSVLTVLHQDWLNGKEYKCKVSNKALPAPIEKTISKAKGQPREPQV | |
| | 351 YTLPPSRDELTKNQVSLTCLVKGFYPSDIAVEWESNGQPENNYKTTPPVL | |
| | 401 DSDGSFFLYSKLTVDKSRWQQGNVFSCSVMHEALHNHYTQKSLSLSPGK | 449 |
| durvalumab | >Heavy chain (SEQ ID NO: 43) | |
| | EVQLVESGGG LVQPGGSLRL SCAASGFTFS RYWMSWVRQA PGKGLEWVAN | 50 |
| | IKQDGSEKYY VDSVKGRFTI SRDNAKNSLY LQMNSLRAED TAVYYCAREG | 100 |
| | GWFGELAFDY WGQGTLVTVS SASTKGPSVF PLAPSSKSTS GGTAALGCLV | 150 |
| | KDYFPEPVTV SWNSGALTSG VHTFPAVLQS SGLYSLSSVV TVPSSSLGTQ | 200 |
| | TYICNVNHKP SNTKVDKRVE PKSCDKTHTC PPCPAPEFEG GPSVFLFPPK | 250 |
| | PKDTLMISRT PEVTCVVVDV SHEDPEVKFN WYVDGVEVHN AKTKPREEQY | 300 |
| | NSTYRVVSVL TVLHQDWLNG KEYKCKVSNK ALPASIEKTI SKAKGQPREP | 350 |
| | QVYTLPPSRE EMTKNQVSLT CLVKGFYPSD IAVEWESNGQ PENNYKTTPP | 400 |
| | VLDSDGSFFL YSKLTVDKSR WQQGNVFSCS VMHEALHNHY TQKSLSLSPG | 450 |
| | K | 451 |
| | >Light chain (SEQ ID NO: 44) | |
| | EIVLTQSPGT LSLSPGERAT LSCRASQRVS SSYLAWYQQK PGQAPRLLIY | 50 |
| | DASSRATGIP DRFSGSGSGT DFTLTISRLE PEDFAVYYCQ QYGSLPWTFG | 100 |
| | QGTKVEIKRT VAAPSVFIFP PSDEQLKSGT ASVVCLLNNF YPREAKVQWK | 150 |
| | VDNALQSGNS QESVTEQDSK DSTYSLSSTL TLSKADYEKH KVYACEVTHQ | 200 |
| | GLSSPVTKSF NRGEC | 215 |
| nivolumab | >Heavy Chain Sequence | |
| | QVQLVESGGGVVQPGRSLRLDCKASGITFSNSGMHWVRQAPG | |
| | KGLEWVAVIWYDGSKRYYADSVKGRFTISRDNSKNTLFLQMN | |
| | SLRAEDTAVYYCATNDDYWGQGTLVTVSSASTKGPSVFPLAPC | |
| | SRSTSESTAALGCLVKDYFPEPVTVSWNSGALTSGVHTFPAVLQ | |
| | SSGLYSLSSVVTVPSSSLGTKTYTCNVDHKPSNTKVDKRVESKY | |
| | GPPCPPCPAPEFLGGPSVFLFPPKPKDTLMISRTPEVTCVVVDVS | |
| | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALHNHYTQ | |
| | KSLSLSLGK (SEQ ID NO: 45) | |
| | >Light Chain Sequence | |
| | EIVLTQSPATLSLSPGERATLSCRASQSVSSYLAWYQQKPGQAP | |
| | RLLIYDASNRATGIPARFSGSGSGTDFTLTISSLEPEDFAVYYCQ | |
| | QSSNWPRTFGQGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | (SEQ ID NO: 46) | |
| pembrolizumab | >Heavy Chain Sequence | |
| | QVQLVQSGVEVKKPGASVKVSCKASGYTFTNYYMYWVRQAP | |
| | GQGLEWMGGINPSNGGTNFNEKFKNRVTLTTDSSTTTAYMELK | |
| | SLQFDDTAVYYCARRDYRFDMGFDYWGQGTTVTVSSASTKGP | |
| | SVFPLAPCSRSTSESTAALGCLVKDYFPEPVTVSWNSGALTSGV | |
| | HTFPAVLQSSGLYSLSSVVTVPSSSLGTKTYTCNVDHKPSNTKV | |
| | DKRVESKYGPPCPPCPAPEFLGGPSVFLFPPKPKDTLMISRTPEV | |
| | TCVVVDVSQEDPEVQFNWYVDGVEVHNAKTKPREEQFNSTYR | |
| | VVSVLTVLHQDWLNGKEYKCKVSNKGLPSSIEKTISKAKGQPR | |
| | EPQVYTLPPSQEEMTKNQVSLTCLVKGFYPSDIAVEWESNGQPE | |
| | NNYKTTPPVLDSDGSFFLYSRLTVDKSRWQEGNVFSCSVMHEA | |
| | LHNHYTQKSLSLSLGK (SEQ ID NO: 47) | |
| | >Light Chain Sequence | |
| | EIVLTQSPATLSLSPGERATLSCRASKGVSTSGYSYLHWYQQKP | |
| | GQAPRLLIYLASYLESGVPARFSGSGSGTDFTLTISSLEPEDFAV | |
| | YYCQHSRDLPLTFGGGTKVEIKRTVAAPSVFIFPPSDEQLKSGTA | |
| | SVVCLLNNFYPREAKVQWKVDNALQSGNSQESVTEQDSKDST | |
| | YSLSSTLTLSKADYEKHKVYACEVTHQGLSSPVTKSFNRGEC | |
| | (SEQ ID NO: 48) | |
| olaratumab | >Heavy chain | |
| | QLQLQESGPG LVKPSETLSL TCTVSGGSIN SSSYYWGWLR QSPGKGLEWI | 50 |
| | GSFFYTGSTY YNPSLRSRLT ISVDTSKNQF SLMLSSVTAA DTAVYYCARQ | 100 |
| | STYYYGSGNY YGWFDRWDQG TLVTVSSAST KGPSVFPLAP SSKSTSGGTA | 150 |
| | ALGCLVKDYF PEPVTVSWNS GALTSGVHTF PAVLQSSGLY SLSSVVTVPS | 200 |
| | SSLGTQTYIC NVNHKPSNTK VDKRVEPKSC DKTHTCPPCP APELLGGPSV | 250 |
| | FLFPPKPKDT LMISRTPEVT CVVVDVSHED PEVKFNWYVD GVEVHNAKTK | 300 |
| | PREEQYNSTY RVVSVLTVLH QDWLNGKEYK CKVSNKALPA PLEKTISKAK | 350 |
| | GQPREPQVYT LPPSREEMTK NQVSLTCLVK GFYPSDIAVE WESNGQPENN | 400 |
| | YKTTPPVLDS DGSFFLYSKL TVDKSRWQQG NVFSCSVMHE ALHNHYTQKS | 450 |
| | LSLSPGK | 457 |
| | (SEQ ID NO: 49) | |
| | >Light chain | |
| | EIVLTQSPAT LSLSPGERAT LSCRASQSVS SYLAWYQQKP GQAPRLLIYD | 50 |
| | ASNRATGIPA RFSGSGSGTD FTLTISSLEP EDFAVYYCQQ RSNWPPAFGQ | 100 |
| | GTKVEIKRTV AAPSVFIFPP SDEQLKSGTA SVVCLLNNFY PREAKVQWKV | 150 |
| | DNALQSGNSQ ESVTEQDSKD STYSLSSTLT LSKADYEKHK VYACEVTHQG | 200 |
| | LSSPVTKSFN RGEC | 214 |
| | (SEQ ID NO: 50) | |
| atezolizumab | >Heavy Chain Sequence | |
| | EVQLVESGGGLVQPGGSLRLSCAASGFTFSDSWIHWVRQAPGK | |
| | GLEWVAWISPYGGSTYYADSVKGRFTISADTSKNTAYLQMNSL | |
| | RAEDTAVYYCARRHWPGGFDYWGQGTLVTVSSASTKGPSVFP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKV | |
| | EPKSCDKTHTCPPCPAPELLGGPSVFLFPPKPKDTLMISRTPEVT | |
| | CVVVDVSHEDPEVKFNWYVDGVEVHNAKTKPREEQYASTYRV | |
| | VSVLTVLHQDWLNGKEYKCKVSNKALPAPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 51) | |
| | >Light Chain Sequence | |
| | DIQMTQSPSSLSASVGDRVTITCRASQDVSTAVAWYQQKPGKA | |
| | PKLLIYSASFLYSGVPSRFSGSGSGTDFTLTISSLQPEDFATYYCQ | |
| | QYLYHPATFGQGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | (SEQ ID NO: 52) | |
| elotuzumab | >Elotuzumab heavy chain | |
| | EVQLVESGGGLVQPGGSLRLSCAASGFDFSRYWMSWVRQAPG | |
| | KGLEWIGEINPDSSTINYAPSLKDKFIISRDNAKNSLYLQMNSLR | |
| | AEDTAVYYCARPDGNYWYFDVWGQGTLVTVSSASTKGPSVFP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKV | |
| | EPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 53) | |
| | >Elotuzumab light chain | |
| | DIQMTQSPSSLSASVGDRVTITCKASQDVGIAVAWYQQKPGKV | |
| | PKLLIYWASTRHTGVPDRFSGSGSGTDFTLTISSLQPEDVATYYC | |
| | QQYSSYPYTFGQGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | (SEQ ID NO: 54) | |
| necitumumab | >Sequence A | |
| | QVQLQESGPGLVKPSQTLSLTCTVSGGSISSGDYYWSWIRQPPG | |
| | KGLEWIGYIYYSGSTDYNPSLKSRVTMSVDTSKNQFSLKVNSVT | |
| | AADTAVYYCARVSIFGVGTFDYWGQGTLVTVSSASTKGPSVLP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKRV | |
| | EPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 55) | |
| | >Sequence A′ | |
| | QVQLQESGPGLVKPSQTLSLTCTVSGGSISSGDYYWSWIRQPPG | |
| | KGLEWIGYIYYSGSTDYNPSLKSRVTMSVDTSKNQFSLKVNSVT | |
| | AADTAVYYCARVSIFGVGTFDYWGQGTLVTVSSASTKGPSVLP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKRV | |
| | EPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 56) | |
| | >Sequence B | |
| | EIVMTQSPATLSLSPGERATLSCRASQSVSSYLAWYQQKPGQAP | |
| | RLLIYDASNRATGIPARFSGSGSGTDFTLTISSLEPEDFAVYYCH | |
| | QYGSTPLTFGGGTKAEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | (SEQ ID NO: 57) | |
| | >Sequence B′ | |
| | EIVMTQSPATLSLSPGERATLSCRASQSVSSYLAWYQQKPGQAP | |
| | RLLIYDASNRATGIPARFSGSGSGTDFTLTISSLEPEDFAVYYCH | |
| | QYGSTPLTFGGGTKAEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | (SEQ ID NO: 58) | |
| daratumumab | Heavy chain | |
| | EVQLLESGGG LVQPGGSLRL SCAVSGFTFN SFAMSWVRQA PGKGLEWVSA | 50 |
| | ISGSGGGTYY ADSVKGRFTI SRDNSKNTLY LQMNSLRAED TAVYFCAKDK | 100 |
| | ILWFGEPVFD YWGQGTLVTV SSASTKGPSV FPLAPSSKST SGGTAALGCL | 150 |
| | VKDYFPEPVT VSWNSGALTS GVHTFPAVLQ SSGLYSLSSV VTVPSSSLGT | 200 |
| | QTYICNVNHK PSNTKVDKRV EPKSCDKTHT CPPCPAPELL GGPSVFLFPP | 250 |
| | KPKDTLMISR TPEVTCVVVD VSHEDPEVKF NWYVDGVEVH NAKTKPREEQ | 300 |
| | YNSTYRVVSV LTVLHQDWLN GKEYKCKVSN KALPAPIEKT ISKAKGQPRE | 350 |
| | PQVYTLPPSR EEMTKNQVSL TCLVKGFYPS DIAVEWESNG QPENNYKTTP | 400 |
| | PVLDSDGSFF LYSKLTVDKS RWQQGNVFSC SVMHEALHNH YTQKSLSLSP | 450 |
| | GK | 452 |
| | (SEQ ID NO: 59) | |
| | Light chain | |
| | EIVLTQSPAT LSLSPGERAT LSCRASQSVS SYLAWYQQKP GQAPRLLIYD | 50 |
| | ASNRATGIPA RFSGSGSGTD FTLTISSLEP EDFAVYYCQQ RSNWPPTFGQ | 100 |
| | GTKVEIKRTV AAPSVFIFPP SDEQLKSGTA SVVCLLNNFY PREAKVQWKV | 150 |
| | DNALQSGNSQ ESVTEQDSKD STYSLSSTLT LSKADYEKHK VYACEVTHQG | 200 |
| | LSSPVTKSFN RGEC | 214 |
| | (SEQ ID NO: 60) | |
| ramucirumab | >9098_H\|ramucirumab\|Homo sapiens\|\|H-GAMMA-1 (VH(1- | |
| | 116) + CH1(117-214) + HINGE-REGION(215-229) + CH2(230- | |
| | 339) + CH3(340-446))\|\|\|\|\|\|\|446\|\|\|\|MW 48696.0\|MW 48696.0\| | |
| | EVQLVQSGGGLVKPGGSLRLSCAASGFTFSSYSMNWVRQAPGK | |
| | GLEWVSSISSSSSYIYYADSVKGRFTISRDNAKNSLYLQMNSLR | |
| | AEDTAVYYCARVTDAFDIWGQGTMVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKVEPK | |
| | SCDKTHTCPPCPAPELLGGPSVFLFPPKPKDTLMISRTPEVTCVV | |
| | VDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK (SEQ ID NO: 61) | |
| | >9098_L\|ramucirumab\|Homo sapiens\|\|L-KAPPA (V-KAPPA(1- | |
| | 107) + C-KAPPA(109-214))\|\|\|\|\|\|\|214\|\|\|\|MW 23124.7\|MW 23124.7\| | |
| | DIQMTQSPSSVSASIGDRVTITCRASQGIDNWLGWYQQKPGKAP | |
| | KLLIYDASNLDTGVPSRFSGSGSGTYFTLTISSLQAEDFAVYFCQ | |
| | QAKAFPPTFGGGTKVDIKGTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | (SEQ ID NO: 62) | |
| dinutuximab | > dinutuximab Heavy chain | |
| | EVQLLQSGPE LEKPGASVMI SCKASGSSFT GYNMNWVRQN IGKSLEWIGA | 50 |
| | IDPYYGGTSY NQKFKGRATL TVDKSSSTAY MHLKSLTSED SAVYYCVSGM | 100 |
| | EYWGQGTSVT VSSASTKGPS VFPLAPSSKS TSGGTAALGC LVKDYFPEPV | 150 |
| | TVSWNSGALT SGVHTFPAVL QSSGLYSLSS VVTVPSSSLG TQTYICNVNH | 200 |
| | KPSNTKVDKR VEPKSCDKTH TCPPCPAPEL LGGPSVFLFP PKPKDTLMIS | 250 |
| | RTPEVTCVVV DVSHEDPEVK FNWYVDGVEV HNAKTKPREE QYNSTYRVVS | 300 |
| | VLTVLHQDWL NGKEYKCKVS NKALPAPIEK TISKAKGQPR EPQVYTLPPS | 350 |
| | REEMTKNQVS LTCLVKGFYP SDIAVEWESN GQPENNYKTT PPVLDSDGSF | 400 |
| | FLYSKLTVDK SRWQQGNVFS CSVMHEALHN HYTQKSLSLS PGK | 443 |
| | (SEQ ID NO: 63) | |
| | > dinutuximab Light chain | |
| | EIVMTQSPAT LSVSPGERAT LSCRSSQSLV HRNGNTYLHW YLQKPGQSPK | 50 |
| | LLIHKVSNRF SGVPDRFSGS GSGTDFTLKI SRVEAEDLGV YFCSQSTHVP | 100 |
| | PLTFGAGTKL ELKRTVAAPS VFIFPPSDEQ LKSGTASVVC LLNNFYPREA | 150 |
| | KVQWKVDNAL QSGNSQESVT EQDSKDSTYS LSSTLTLSKA DYEKHKVYAC | 200 |
| | EVTHQGLSSP VTKSFNRGEC | 220 |
| | (SEQ ID NO: 64) | |
| | > dinutuximab beta Heavy chain | |
| | EVQLLQSGPE LEKPGASVMI SCKASGSSFT GYNMNWVRQN IGKSLEWIGA | 50 |
| | IDPYYGGTSY NQKFKGRATL TVDKSSSTAY MHLKSLTSED SAVYYCVSGM | 100 |
| | EYWGQGTSVT VSSASTKGPS VFPLAPSSKS TSGGTAALGC LVKDYFPEPV | 150 |
| | TVSWNSGALT SGVHTFPAVL QSSGLYSLSS VVTVPSSSLG TQTYICNVNH | 200 |
| | KPSNTKVDKR VEPKSCDKTH TCPPCPAPEL LGGPSVFLFP PKPKDTLMIS | 250 |
| | RTPEVTCVVV DVSHEDPEVK FNWYVDGVEV HNAKTKPREE QYNSTYRVVS | 300 |
| | VLTVLHQDWL NGKEYKCKVS NKALPAPIEK TISKAKGQPR EPQVYTLPPS | 350 |
| | REEMTKNQVS LTCLVKGFYP SDIAVEWESN GQPENNYKTT PPVLDSDGSF | 400 |
| | FLYSKLTVDK SRWQQGNVFS CSVMHEALHN HYTQKSLSLS PGK | 443 |
| | (SEQ ID NO: 65) | |
| | >dinutuximab beta Light chain | |
| | EIVMTQSPAT LSVSPGERAT LSCRSSQSLV HRNGNTYLHW YLQKPGQSPK | 50 |
| | LLIHKVSNRF SGVPDRFSGS GSGTDFTLKI SRVEAEDLGV YFCSQSTHVP | 100 |
| | PLTFGAGTKL ELKRTVAAPS VFIFPPSDEQ LKSGTASVVC LLNNFYPREA | 150 |
| | KVQWKVDNAL QSGNSQESVT EQDSKDSTYS LSSTLTLSKA DYEKHKVYAC | 200 |
| | EVTHQGLSSP VTKSFNRGEC | 220 |
| | (SEQ ID NO: 66) | |
| blinatumomab | >single chain variable fragment fusion protein | |
| | DIQLTQSPASLAVSLGQRATISCKASQSVDYDGDSYLNWYQQIP | |
| | GQPPKLLIYDASNLVSGIPPRFSGSGSGTDFTLNIHPVEKVDAAT | |
| | YHCQQSTEDPWTFGGGTKLEIKGGGGSGGGGSGGGGSQVQLQ | |
| | QSGAELVRPGSSVKISCKASGYAFSSYWMNWVKQRPGQGLEW | |
| | IGQIWPGDGDTNYNGKFKGKATLTADESSSTAYMQLSSLASED | |
| | SAVYFCARRETTTVGRYYYAMDYWGQGTTVTVSSGGGGSDIK | |
| | LQQSGAELARPGASVKMSCKTSGYTFTRYTMHWVKQRPGQGL | |
| | EWIGYINPSRGYTNYNQKFKDKATLTTDKSSSTAYMQLSSLTSE | |
| | DSAVYYCARYYDDHYCLDYWGQGTTLTVSSVEGGSGGSGGSG | |
| | GSGGVDDIQLTQSPAIMSASPGEKVTMTCRASSSVSYMNWYQQ | |
| | KSGTSPKRWIYDTSKVASGVPYRFSGSGSGTSYSLTISSMEAED | |
| | AATYYCQQWSSNPLTFGAGTKLELKHHHHHH | |
| | (SEQ ID NO: 67) | |
| siltuximab | >Heavy Chain Sequence | |
| | EVQLVESGGKLLKPGGSLKLSCAASGFTFSSFAMSWFRQSPEKR | |
| | LEWVAEISSGGSYTYYPDTVTGRFTISRDNAKNTLYLEMSSLRS | |
| | EDTAMYYCARGLWGYYALDYWGQGTSVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVE | |
| | PKSCDKTHTCPPCPAPELLGGPSVFLFPPKPKDTLMISRTPEVTC | |
| | VVVDVSHEDPEVKFNWYVDGVEVHNAKTKPREEQYNSTYRV | |
| | VSVLTVLHQDWLNGKEYKCKVSNKALPAPIEKTISKAKGQPRE | |
| | PQVYTLPPSRDELTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK (SEQ ID NO: 68) | |
| | >Light Chain Sequence | |
| | QIVLIQSPAIMSASPGEKVTMTCSASSSVSYMYWYQQKPGSSPR | |
| | LLIYDTSNLASGVPVRFSGSGSGTSYSLTISRMEAEDAATYYCQ | |
| | QWSGYPYTFGGGTKLEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | (SEQ ID NO: 69) | |
| denosumab | >Denosumab αOPGL-1 heavy chain sequence | |
| | EVQLLESGGGLVQPGGSLRLSCAASGFTFSSYAMSWVRQAPGK | |
| | GLEWVSGITGSGGSTYYADSVKGRFTISRDNSKNTLYLQMNSL | |
| | RAEDTAVYYCAKDPGTTVIMSWFDPWGQGTLVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVD | |
| | KKVEPKSCDKTHTCPPCPAPELLGGPSVFLFPPKPKDTLMISRTP | |
| | EVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNGQ | |
| | PENNYKTTPPVLDSDGSFFLYSKLTVDKSRWQQGNVFSCSVMH | |
| | EALHNHYTQKSLSLSPGK (SEQ ID NO: 70) | |
| | >Denosumab αOPGL-1 light chain sequence | |
| | EIVLTQSPGTLSLSPGERATLSCRASQSVRGRYLAWYQQKPGQA | |
| | PRLLIYGASSRATGIPDRFSGSGSGTDFTLTISRLEPEDFAVFYCQ | |
| | QYGSSPRTFGQGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | (SEQ ID NO: 71) | |
| Enoblituzumab | >Heavy chain | |
| | QVELVESGGG LVQPGGSLRL SCAASGFTFT SYWMSWVRQA PGKGLELVSS | 50 |
| | ITSYGSFTYY ADSVKGRFTI SRDNSKNTLY LQMNSLRAED TAVYYCARNM | 100 |
| | YTHFDSWGQG TLVTVSSAST KGPSVFPLAP SSKSTSGGTA ALGCLVKDYF | 150 |
| | PEPVTVSWNS GALTSGVHTF PAVLQSSGLY SLSSVVTVPS SSLGTQTYIC | 200 |
| | NVNHKPSNTK VDKKVEPKSC DKTHTCPPCP APELLGGPSV FLFPPKPKDT | 250 |
| | LMISRTPEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT | 350 |
| | LPPSRDELTK NQVSLTCLVK GFYPSDIAVE WESNGQPENN YKTTPPVLDS | 400 |
| | DGSFFLYSKL TVDKSRWQQG NVFSCSVMHE ALHNHYTQKS LSLSPGK | 447 |
| | (SEQ ID NO: 72) | |
| | >Light chain | |
| | DIVLTQPPSV SGAPGQRVTI SCSGSSSNIG SNSVSWYQQL PGTAPKLLIY | 50 |
| | DNSKRPSGVP DRFSGSKSGT SASLAITGLQ SEDEADYYCQ SRDTYGYYWV | 100 |
| | FGGGTKLTVL GQPKAAPSVT LFPPSSEELQ ANKATLVCLI SDFYPGAVTV | 150 |
| | AWKGDSSPVK AGVETTTPSK QSNNKYAASS YLSLTPEQWK SHRSYSCQVT | 200 |
| | HEGSTVEKTV APTECS | 216 |
| | (SEQ ID NO: 73) | |
| Lirilumab | >Heavy chain | |
| | QVQLVQSGAE VKKPGSSVKV SCKASGGTFS FYAISWVRQA PGQGLEWMGG | 50 |
| | FIPIFGAANY AQKFQGRVTI TADESTSTAY MELSSLRSDD TAVYYCARIP | 100 |
| | SGSYYYDYDM DVWGQGTTVT VSSASTKGPS VFPLAPCSRS TSESTAALGC | 150 |
| | LVKDYFPEPV TVSWNSGALT SGVHTFPAVL QSSGLYSLSS VVTVPSSSLG | 200 |
| | TKTYTCNVDH KPSNTKVDKR VESKYGPPCP PCPAPEFLGG PSVFLFPPKP | 250 |
| | KDTLMISRTP EVTCVVVDVS QEDPEVQFNW YVDGVEVHNA KTKPREEQFN | 300 |
| | STYRVVSVLT VLHQDWLNGK EYKCKVSNKG LPSSIEKTIS KAKGQPREPQ | 350 |
| | VYTLPPSQEE MTKNQVSLTC LVKGFYPSDI AVEWESNGQP ENNYKTTPPV | 400 |
| | LDSDGSFFLY SRLTVDKSRW QEGNVFSCSV MHEALHNHYT QKSLSLSLGK | 450 |
| | (SEQ ID NO: 74) | |
| | >Light chain | |
| | EIVLTQSPVT LSLSPGERAT LSCRASQSVS SYLAWYQQKP GQAPRLLIYD | 50 |
| | ASNRATGIPA RFSGSGSGTD FTLTISSLEP EDFAVYYCQQ RSNWMYTFGQ | 100 |
| | GTKLEIKRTV AAPSVFIFPP SDEQLKSGTA SVVCLLNNFY PREAKVQWKV | 150 |
| | DNALQSGNSQ ESVTEQDSKD STYSLSSTLT LSKADYEKHK VYACEVTHQG | 200 |
| | LSSPVTKSFN RGEC | 214 |
| | (SEQ ID NO: 75) | |
| pidilizumab | >Heavy chain | |
| | QVQLVQSGSE LKKPGASVKI SCKASGYTFT NYGMNWVRQA PGQGLQWMGW | 50 |
| | INTDSGESTY AEEFKGRFVF SLDTSVNTAY LQITSLTAED TGMYFCVRVG | 100 |
| | YDALDYWGQG TLVTVSSAST KGPSVFPLAP SSKSTSGGTA ALGCLVKDYF | 150 |
| | PEPVTVSWNS GALTSGVHTF PAVLQSSGLY SLSSVVTVPS SSLGTQTYIC | 200 |
| | NVNHKPSNTK VDKRVEPKSC DKTHTCPPCP APELLGGPSV FLFPPKPKDT | 250 |
| | LMISRTPEVT CVVVDVSHED PEVKFNWYVD GVEVHNAKTK PREEQYNSTY | 300 |
| | RVVSVLTVLH QDWLNGKEYK CKVSNKALPA PLEKTISKAK GQPREPQVYT | 350 |
| | LPPSREEMTK NQVSLTCLVK GFYPSDIAVE WESNGQPENN YKTTPPVLDS | 400 |
| | DGSFFLYSKL TVDKSRWQQG NVFSCSVMHE ALHNHYTQKS LSLSPGK | 447 |
| | (SEQ ID NO: 76) | |
| | >Light chain | |
| | EIVLTQSPSS LSASVGDRVT ITCSARSSVS YMHWFQQKPG KAPKLWIYRT | 50 |
| | SNLASGVPSR FSGSGSGTSY CLTINSLQPE DFATYYCQQR SSFPLTFGGG | 100 |
| | TKLEIKRTVA APSVFIFPPS DEQLKSGTAS VVCLLNNFYP REAKVQWKVD | 150 |
| | NALQSGNSQE SVTEQDSKDS TYSLSSTLTL SKADYEKHKV YACEVTHQGL | 200 |
| | SSPVTKSFNR GEC | 213 |
| | (SEQ ID NO: 77) | |
| avelumab | >Heavy chain | |
| | EVQLLESGGG LVQPGGSLRL SCAASGFTFS SYIMMWVRQA PGKGLEWVSS | 50 |
| | IYPSGGITFY ADTVKGRFTI SRDNSKNTLY LQMNSLRAED TAVYYCARIK | 100 |
| | LGTVTTVDYW GQGTLVTVSS ASTKGPSVFP LAPSSKSTSG GTAALGCLVK | 150 |
| | DYFPEPVTVS WNSGALTSGV HTFPAVLQSS GLYSLSSVVT VPSSSLGTQT | 200 |
| | YICNVNHKPS NTKVDKKVEP KSCDKTHTCP PCPAPELLGG PSVFLFPPKP | 250 |
| | KDTLMISRTP EVTCVVVDVS HEDPEVKFNW YVDGVEVHNA KTKPREEQYN | 300 |
| | STYRVVSVLT VLHQDWLNGK EYKCKVSNKA LPAPLEKTIS KAKGQPREPQ | 350 |
| | VYTLPPSRDE LTKNQVSLTC LVKGFYPSDI AVEWESNGQP ENNYKTTPPV | 400 |
| | LDSDGSFFLY SKLTVDKSRW QQGNVFSCSV MHEALHNHYT QKSLSLSPGK | 450 |
| | (SEQ ID NO: 78) | |
| | >Light chain | |
| | QSALTQPASV SGSPGQSITI SCTGTSSDVG GYNYVSWYQQ HPGKAPKLMI | 50 |
| | YDVSNRPSGV SNRFSGSKSG NTASLTISGL QAEDEADYYC SSYTSSSTRV | 100 |
| | FGTGTKVTVL GQPKANPTVT LFPPSSEELQ ANKATLVCLI SDFYPGAVTV | 150 |
| | AWKADGSPVK AGVETTKPSK QSNNKYAASS YLSLTPEQWK SHRSYSCQVT | 200 |
| | HEGSTVEKTV APTECS | 216 |
| | (SEQ ID NO: 79) | |

TABLE 3
Exemplary cancers

| | | | |
|---|---|---|---|
| Acute | Colorectal cancer | Macroglobulinemia, | Pleuropulmonary |
| lymphoblastic | | Waldenström | Blastoma, |
| leukaemia (ALL) | | | Childhood |
| Acute myeloid | Craniopharyngioma, | Male Breast Cancer | Pregnancy and |
| leukaemia | Childhood | | Breast Cancer |
| (AML) | | | |
| Adrenocortical | Cutaneous T-Cell | Malignant Fibrous Histiocytoma | Primary Central |
| Carcinoma | Lymphoma | of Bone and Osteosarcoma | Nervous System |
| | | | (CNS) Lymphoma |
| AIDS-Related | Ductal Carcinoma In Situ | Melanoma | Prostate Cancer |
| Kaposi Sarcoma | (DCIS) | | |
| AIDS-Related | Embryonal Tumors, | Merkel Cell Carcinoma | Rare cancers |
| lymphoma | Childhood | | |
| Anal Cancer | Endometrial Cancer | Mesothelioma | Rectal Cancer |
| Appendix Cancer | Ependymoma, Childhood | Metastatic Squamous Neck | Renal cell |
| | | Cancer with Occult Primary | carcinoma |
| Astrocytomas, | Epithelial cancer | Midline Tract Carcinoma | Renal Pelvis and |
| Childhood | | Involving NUT Gene | Ureter, |
| | | | Transitional Cell |
| | | | Cancer |
| Atypical | Esophageal Cancer | Molar pregnancy | Retinoblastoma |
| Teratoid/Rhabdoid | | | |
| Tumor, Childhood | | | |
| Basal Cell | Esthesioneuroblastoma, | Mouth and oropharyngeal cancer | Rhabdomyosarcoma |
| Carcinoma | Childhood | | |
| Bile duct cancer | Ewing sarcoma | Multiple Endocrine Neoplasia | Salivary Gland |
| | | Syndromes, Childhood | Cancer |
| Bladder cancer | Extragonadal Germ Cell | Multiple Myeloma/Plasma Cell | Sarcoma |
| | Tumor | Neoplasm | |
| Bone cancer | Extrahepatic Bile Duct | Mycosis Fungoides | Secondary cancers |
| | Cancer | | |
| Bowel cancer | Eye Cancer | Myelodysplastic Syndromes | Sézary Syndrome |
| Brain Stem | Gallbladder Cancer | Myelodysplastic/Myeloproliferative | Skin Cancer |
| Glioma, | | Neoplasms | |
| Childhood | | | |
| Brain tumors | Gastric cancer | Myeloproliferative Disorders, | Skin cancer (non |
| | | Chronic | melanoma) |
| Breast cancer | Gastrointestinal Carcinoid | Nasal Cavity and Paranasal Sinus | Small Cell Lung |
| | Tumor | Cancer | Cancer |
| Bronchial | Germ Cell Tumor | Nasopharyngeal cancer | Small Intestine |
| Tumors, | | | Cancer |
| Childhood | | | |
| Burkitt | Gestational trophoblastic | Neuroblastoma | Soft Tissue |
| Lymphoma | tumors (GTT) | | Sarcoma |
| Cancer of | Glioma | Non-Hodgkin Lymphoma | Squamous Cell |
| unknown primary | | | Carcinoma |
| Cancer spread to | Hairy cell leukaemia | Non-Small Cell Lung Cancer | Squamous Neck |
| bone | | | Cancer with Occult |
| | | | Primary, |
| | | | Metastatic |
| Cancer spread to | Head and neck cancer | Oesophageal cancer | Stomach (Gastric) |
| brain | | | Cancer |
| Cancer spread to | Heart Cancer, Childhood | Oral Cancer | Stomach cancer |
| liver | | | |
| Cancer spread to | Hepatocellular (Liver) Cancer | Oral Cavity Cancer | T-Cell Lymphoma, Cutaneous - see |
| lung | | | Mycosis |
| | | | Fungoides and |
| | | | Sézary Syndrome |
| Carcinoid Tumor | Histiocytosis, Langerhans | Oropharyngeal Cancer | Testicular cancer |
| | Cell | | |
| Carcinoma of | Hodgkin Lymphoma | Osteosarcoma (Bone Cancer) | Throat Cancer |
| Unknown | | | |
| Primary | | | |
| Cardiac (Heart) | Hypopharyngeal Cancer | Osteosarcoma and Malignant | Thymoma and |
| Tumors, | | Fibrous Histiocytoma | Thymic Carcinoma |
| Childhood | | | |
| Central Nervous | Intraocular Melanoma | Ovarian Cancer | Thyroid Cancer |
| System Atypical | | | |
| Teratoid/Rhabdoid | | | |
| Tumor, | | | |
| Childhood | | | |
| Central Nervous | Islet Cell Tumors, | Pancreatic Cancer | Transitional Cell |
| System | Pancreatic Neuroendocrine | | Cancer of the |
| Embryonal | Tumors | | Renal Pelvis and |
| Tumors, | | | Ureter |
| Childhood | | | |
| Central Nervous | Kidney cancer | Pancreatic Neuroendocrine | Unknown primary |
| System, | | Tumors (Islet Cell Tumors) | cancer |
| Childhood | | | |
| Cervical cancer | Langerhans Cell | Papillomatosis, Childhood | Ureter and Renal |
| | Histiocytosis | | Pelvis, Transitional |
| | | | Cell Cancer |
| Chordoma, | Laryngeal Cancer | Paraganglioma | Urethral Cancer |
| Childhood | | | |
| Choriocarcinoma | Leukemia | Parathyroid Cancer | Uterine Cancer, |
| | | | Endometrial |
| Chronic | Lip and Oral Cavity | Penile Cancer | Uterine Sarcoma |
| Lymphocytic | Cancer | | |
| Leukemia (CLL) | | | |
| Chronic myeloid | Liver cancer | Pharyngeal Cancer | Vaginal cancer |
| leukaemia | | | |
| (CML) | | | |
| Chronic | Lobular Carcinoma In Situ | Pheochromocytoma | Vulvar Cancer |
| Myeloproliferative | (LCIS) | | |
| Disorders | | | |
| Colon cancer | Low Malignant Potential | Pituitary Tumor | Waldenström |
| | Tumor | | Macroglobulinemia |
| Lymphoma | Lung Cancer | Plasma Cell Neoplasm/Multiple | Wilms Tumor |
| | | Myeloma | |

TABLE 4
Immune checkpoint molecules, and agents that bind thereto.

| | Antibody or other binding agent (e.g., immune |
|---|---|
| Immune checkpoint molecule | checkpoint molecule inhibitors) |
| A2aR | Anti-Adenosine Receptor A2a Antibody, clone 7F6-G5-A2 |
| | (EMD Millipore) |
| 2B4 (also called CD244 or | CD48 (e.g., Accession Number CAG33293.1) |
| SLAMF4) | |
| B-7 family ligand, e.g., B7-H3 | Enoblituzumab |
| B7-H4 | h1D11 TDC (see Leong et al., doi: 10.1021/mp5007745) |
| BTLA | HVEM (e.g., Accession Number AAQ89238.1) |
| CGEN-15049 | Anti- CGEN-15049 antibody |
| CD160 | HVEM (e.g., Accession Number AAQ89238.1) |
| CEACAM (e.g., CEACAM- 1, | Annexin, e.g., Annexin A2 (e.g., Accession Number |
| CEACAM-3 and/or CEACAM-5) | AAH52558.1) |
| CHK 1 | 2G1D5 mAb (Cell Signalling Technology) |
| CHK2 | 1C12 mAb #3440 (Cell Signalling Technology) |
| CTLA4 | B7-1 (CD80; e.g., Accession Number NP_005182.1); B7-2 |
| | (CD86; e.g., Accession Number NP_787058.4); |
| | ipilimumab, tremelimumab |
| GAL9 | Anti-human Galectin-9 Antibody 9M1-3 (BioLegend) |
| GR1 | RB6-8C5 antibody (eBioscience) |
| HVEM | BTLA (e.g., Accession Number NP_861445.3), CD160 |
| | (e.g., Accession Number NP_008984.1) |
| KIR | Lirilumab |
| LAG3 | BMS-986016 |
| LAIR1 | COLLAGEN; Anti-Human CD305 (eBioscience) |
| LY6G | 1A8 antibody (eBioscience) |
| MUC1 | Anti-MUC1 antibody EPR1023 (Abcam) |
| PD1 | B7DC; PD-L1; nivolumab; pembrolizumab; pidilizumab |
| PD-L1 (also called B7H1) | B7-1 (CD80; e.g., Accession Number NP_005182.1); PD1 |
| | (e.g., Accession Number NP_005009.2); durvalumab; |
| | atezolizumab; avelumab; BMS 936559 |
| PDL2 | AMP 224 |
| TGF beta | Antibody 1D11 (see Ueda R et al., doi: 10.1158/1078- |
| | 0432.CCR-09-1067) |
| TIGIT | CD155 (e.g., Accession Number NP_001129240.1); CD112 |
| | (e.g., Accession Number NP_001036189.1); CD113 (e.g., |
| | Accession Number NP_001230215.1); |
| TIM3 | GALECTIN9; |
| TIM4 | TIM4R; CD48 (e.g., Accession Number NP_001769.2) |
| TIM4R | TIM4 (e.g., Accession Number NP_612388.2) |
| VISTA | Human VISTA Antibody Clone # 730804 (R&D Systems) |

TABLE 5
Costimulatory molecules, and agents that bind thereto.

| Costimulatory molecule | Antibody or other binding agent |
|---|---|
| 4-1BB (CD137; e.g., Accession | 4-1BBL (e.g., Accession Number NP_003802.1), CD137L |
| NP_001552.2) | (e.g., Accession Number NP_003802.1) |
| CD2 (e.g., Accession Number | CD48 (e.g., Accession Number NP_001769.2) |
| NP_001315538.1) | |
| CD27 (e.g., Accession Number | CD70 (e.g., Accession Number NP_001243.1) |
| NP_001233.1) | |
| CD28 (e.g., Accession Number | B7-H2 (e.g., Accession Number NP_056074.1), B7-1 |
| NP_006130.1) | (CD80; e.g., Accession Number NP_005182.1) |
| CD30 (e.g., Accession Number | CD30L (e.g., Accession Number NP_001235.1), |
| NP_001234.3) | brentuximab |
| CD40 (e.g., Accession Number | CD40L (e.g., Accession Number BAA06599.1) |
| NP_001241.1) | |
| CD226 (e.g., Accession Number | CD155 (e.g., Accession Number NP_001129240.1), CD112 |
| NP_006557.2) | (e.g., Accession Number NP_001036189.1) |
| CDS | |
| DR3 (e.g., Accession Number | TL1A (e.g., Accession Number NP_005109.2) |
| NP_683866.1) | |
| GITR (e.g., Accession Number | GITRL (e.g., Accession Number NP_005083.2) |
| NP_004186.1) | |
| HVEM (LIGHTR) (e.g., | LIGHT (e.g., Accession Number NP_003798.2) |
| Accession Number AAQ89238.1) | |
| ICAM-1 (e.g., Accession Number | Anti-ICAM1 antibody [MEM-111] (Abcam) |
| NP_000192.2) | |
| LFA-1 (CD11a/CD18) (e.g., | odulimomab |
| Accession Number NP_002200.2) | |
| LIGHT (e.g., Accession Number | HVEM (e.g., Accession Number AAQ89238.1) |
| NP_003798.2) | |
| ICOS (CD278) (e.g., Accession | B7-H2 (e.g., Accession Number NP_056074.1) |
| Number NP_036224.1) | |
| OX40 (e.g., Accession Number NP_003318.1) | OX40L (e.g., Accession Number NP_003317.1) |
| TIM1 (e.g., Accession Number | TIM4 (e.g., Accession Number NP_612388.2) |
| NP_036338.2) | |

TABLE 6
αCD20-RCTs binding to CD20 cell lines; Percent cells bound

| | αCD20- | HA-GPA control |
|---|---|---|
| Cell type | RCT binding | RCT binding |
| Raji | 83.6% | 7.2% |
| Ramos | 83.0% | 5.59% |
| Z138 | 43.7% | 3.92% |
| Granta-519 | 76.0% | 8.95% |
| Toledo | 37.1% | 5.96% |
| SU-DHL4 | 91.3% | 36.1% |
| DOHH-2 | 86.7% | 7.18% |

TABLE 7
BCL-xL and BCL-2 levels in cancer cells exposed
to RCT-antiCD20 or control RCT-HA-GPA

| Cell Type | RCT-antiCD20 | RCT-HA-GPA |
|---|---|---|
| Raji (1:1 Ratio) | BCL-xL+/BCL-2+: 93.9% | BCL-xL+/BCL-2+: 98.5% |
| | BCL-xL−/BCL-2+: 1.58% | BCL-xL−/BCL-2+: 0.20 |
| | BCL-xL−/BCL-2−: 3.46% | BCL-xL−/BCL-2−: 0.60 |
| | BCL-xL+/BCL-2−: 1.06% | BCL-xL+/BCL-2−: 0.67% |
| Raji (1: 5 Ratio) | BCL-xL+/BCL-2+: 69.8% | BCL-xL+/BCL-2+: 98.9% |
| | BCL-xL−/BCL-2+: 22.2% | BCL-xL−/BCL-2+: 0.50% |
| | BCL-xL−/BCL-2−: 7.27% | BCL-xL−/BCL-2−: 0.50% |
| | BCL-xL+/BCL-2−: 0.76% | BCL-xL+/BCL-2−: 0.14% |
| Ramos (1: 1 Ratio) | BCL-xL+/BCL-2+: 79.1% | BCL-xL+/BCL-2+: 89.0% |
| | BCL-xL−/BCL-2+: 4.56% | BCL-xL−/BCL-2+: 0.94% |
| | BCL-xL−/BCL-2−: 5.08% | BCL-xL−/BCL-2−: 2.31% |
| | BCL-xL+/BCL-2−: 11.2% | BCL-xL+/BCL-2−: 7.72% |
| Ramos (1: 5 Ratio) | BCL-xL+/BCL-2+: 86.5% | BCL-xL+/BCL-2+: 98.9% |
| | BCL-xL−/BCL-2+: 9.83% | BCL-xL−/BCL-2+: 0.67% |
| | BCL-xL−/BCL-2−: 1.91% | BCL-xL−/BCL-2−: 0.26% |
| | BCL-xL+/BCL-2−: 1.79% | BCL-xL+/BCL-2−: 0.21% |
| DOHH-2 (1: 1 Ratio) | BCL-xL+/BCL-2+: 88.9% | BCL-xL+/BCL-2+: 98.6% |
| | BCL-xL−/BCL-2+: 8.88% | BCL-xL−/BCL-2+: 1.01% |
| | BCL-xL−/BCL-2−: 1.73% | BCL-xL−/BCL-2−: 0.26% |
| | BCL-xL+/BCL-2−: 0.52% | BCL-xL+/BCL-2−: 0.099% |
| DOHH-2 (1: 5 Ratio) | BCL-xL+/BCL-2+: 74.4% | BCL-xL+/BCL-2+: 99.2% |
| | BCL-xL−/BCL-2+: 24.4% | BCL-xL−/BCL-2+: 0.77% |
| | BCL-xL−/BCL-2−: 1.12% | BCL-xL−/BCL-2−: 0.072% |
| | BCL-xL+/BCL-2−: 0.023% | BCL-xL+/BCL-2−: 0.0016% |
| SU-DHL4 (1: 1 Ratio) | BCL-xL+/BCL-2+: 95.1% | BCL-xL+/BCL-2+: 98.8% |
| | BCL-xL−/BCL-2+: 0.32% | BCL-xL−/BCL-2+: 0.74% |
| | BCL-xL1/BCL-2−: 1.11% | BCL-xL−/BCL-2−: 0.37% |
| | BCL-xL+/BCL-2−: 3.49% | BCL-xL+/BCL-2−: 0.14% |
| SU-DHL4 (1: 5 Ratio) | BCL-xL+/BCL-2+: 93.0% | BCL-xL+/BCL-2+: 98.3% |
| | BCL-xL−/BCL-2+: 5.54% | BCL-xL−/BCL-2+: 1.13% |
| | BCL-xL−/BCL-2−: 1.14% | BCL-xL−/BCL-2−: 0.46% |
| | BCL-xL+/BCL-2−: 0.33% | BCL-xL+/BCL-2−: 0.085% |

TABLE 8
Genes mutated in certain cancers

| Gene | Exemplary cancers | Exemplary mutations |
|---|---|---|
| ABCB1 | AML; breast cancer | 3853C > T |
| ABCB1 | AML | 2677G > T/A |
| ABCC2 | | |
| ABCC4 | | |
| ABCG2 | | q141K |
| ABL1 | Leukemia (e.g., chronic myeloid leukemia | Codon 315 |
| | (CML), acute myeloid leukemia (AML), | |
| | acute lymphoblastic leukemia (ALL)) | |
| ABL1 | CML, ALL, T-ALL | Translocation with BCR, ETV6, NUP214 |
| ABL2 | AML | Translocation with ETV6 |
| ACSL3 | prostate | Translocation with ETV1 |
| ACVR1B | Pancreas, breast | |
| AF15Q14 | AML | Translocation with MLL |
| AF1Q | ALL | Translocation with MLL |
| AF3p21 | ALL | Translocation with MLL |
| AF5q31 | ALL | Translocation with MLL |
| AKAP9 | papillary thyroid | Translocation with BRAF |
| AKT1 | breast cancer, colorectal cancer, ovarian | |
| | cancer | |
| AKT2 | Ovarian cancer, pancreatic cancer | |
| AKT3 | Melanoma, glioma, uternine cancer, prostate | |
| | cancer, oral cancer, | |
| | ovarian cancer | |
| ALK | ALCL, NSCLC, Neuroblastoma, Lymphoma | Translocation with NPM1, TPM3, TFG, |
| | (e.g., non-Hodgkin lymphoma, anaplastic | TPM4, ATIC, CLTC, MSN, ALO17, CARS, |
| | large-cell lymphoma (ALCL)), inflammatory | or EML4 |
| | myofibroblastic tumor | |
| ALK | ALCL, NSCLC, Neuroblastoma, Lymphoma | Translocation with NPM1, TPM3, TFG, |
| | (e.g., non-Hodgkin lymphoma, anaplastic | TPM4, ATIC, CLTC, MSN, ALO17, CARS, |
| | large-cell lymphoma (ALCL)), inflammatory | or EML4 |
| | myofibroblastic tumor | |
| ALO17 | ALCL | Translocation with ALK |
| ALOX12B | Mutiple myeloma (MM) | |
| APC | Colorectal cancer, medulloblastoma, | Codon 1114, 1338, 1450, or 1556 |
| | mismatch repair cancer syndrome | |
| AR | Prostate cancer | |
| ARAF | Angioimmunoblastic T-cell lymphoma, | |
| | ehrlich ascites tumor | |
| ARFRP1 | Breast cancer | |
| ARHGEF12 | AML | Translocation with MLL |
| ARHH | NHL | Translocation with BCL6 |
| ARID1A | Neuroblastoma, acute lymphoblastic | |
| | leukemia (ALL), neuroendocrine tumor | |
| ARNT | AML | Translocation with ETV6 |
| ASPSCR1 | alveolar soft part sarcoma | Translocation with TFE3 |
| ASXL1 | Mutiple myeloma (MM) | |
| ATF1 | malignant melanoma of soft parts, | Translocation with EWSR1 or FUS |
| | angiomatoid fibrous histiocytoma | |
| ATIC | ALCL | Translocation with ALK |
| ATM | Leukemia (e.g., T-cell prolymphocytic | Biallelic inactivation |
| | leukemia (T-PLL)), lymphoma, | |
| | medulloblastoma, glioma, breast cancer | |
| ATR | Pyothorax-associated lymphoma, T-cell | Biallelic inactivation |
| | lymphoma, breast cancer | |
| ATRX | Pancreatic neuroendocrine tumors | |
| AURKA | Laryngeal squamous cell carcinoma, ovarian | |
| | cancer, bladder cancer, head and neck | |
| | squamous cell carcinoma (HNSCC), | |
| | laryngeal carcinoma, esophageal squamous | |
| | cell carcinoma (ESCC), pancreatic cancer | |
| AURKB | Colorectal cancer, astrocytoma, ependymal | |
| | tumor, glioma, esophageal squamous cell | |
| | carcinoma (ESCC), acute myeloid leukemia | |
| | (AML) | |
| AXL | Non small cell lung cancer (NSCLC), MM | |
| BACH1 | Breast | |
| BAP1 | Uveal melanoma, breast, NSCLC | |
| BARD1 | Breast | |
| BCL10 | MALT | Translocation with IGH |
| BCL11A | B-CLL | Translocation with IGH |
| BCL11B | T-ALL | Translocation with TLX3 |
| BCL2 | Lymphoma, colorectal adenocarcinoma, | |
| | esophageal squamous cell carcinoma | |
| | (ESCC), synovial sarcoma, leukemia | |
| BCL2 | NHL, CLL | Translocation with IGH |
| BCL2A1 | Pulmonary granuloma, gastric adenoma, | |
| | burkitt lymphoma, parotid adenoma, kaposi | |
| | sarcoma, gastric cancer, colon cancer | |
| BCL2L1 | Head and neck squamous cell carcinoma, | |
| | glioblastoma, mesothelioma, pancreatic | |
| | cancer, adenocarcinoma lung | |
| BCL2L2 | Brain cancer, leukemia, lymphoma, | |
| | colorectal adenocarcinoma, colorectal | |
| | cancer, adenoma, cervical squamous cell | |
| | carcinoma | |
| BCL3 | CLL | Translocation with IGH |
| BCL5 | CLL | Translocation with MYC |
| BCL6 | Lymphoma, leukemia | |
| BCL6 | NHL, CLL | Translocation with IG loci, ZNFN1A1, |
| | | LCP1, PIM1, TFRC, MHC2TA, NACA, HSPCB, |
| | | HSPCA, HIST1H4I, IL21R, POU2AF1, ARHH, EIF4A2, |
| | | or SFRS3 |
| BCL7A | BNHL | Translocation with MYC |
| BCL9 | B-ALL | Translocation with IGH or IGL |
| BCOR | Breast | |
| BCORL1 | Breast | |
| BCR | CML, ALL, AML | Translocation with ABL1, FGFR1, or JAK2 |
| BIRC3 | MALT | Translocation with MALT1 |
| BLM | Leukemia, lymphoma, skin squamous cell, | |
| | other cancers | |
| BRAF | Lung cancer, non-Hodgkin lymphoma, | Codon 594 (e.g., D594G) or 600 (e.g., V600E) |
| | colorectal cancer, thyroid cancer, melanoma, | |
| | breast cancer | |
| BRAF | melanoma, colorectal, papillary thyroid, | Translocation with AKAP9 or KIAA1549 |
| | borderline ov, Non small-cell lung cancer | |
| | (NSCLC), cholangiocarcinoma, pilocytic | |
| | astrocytoma | |
| BRCA1 | Breast cancer, ovarian cancer | Biallelic inactivation |
| BRCA2 | Breast cancer, ovarian cancer, pancreatic | Biallelic inactivation |
| | cancer | |
| BRD3 | lethal midline carcinoma of young people | Translocation with NUT |
| BRD4 | lethal midline carcinoma of young people | Translocation with NUT |
| BRIP1 | Acute myeloid leukemia (AML), leukemia, | |
| | breast | |
| BTG1 | BCLL | Translocation with MYC |
| C12orf9 | lipoma | Translocation with LPP |
| C15orf21 | prostate | Translocation with ETV1 |
| C17orf39 | Breast | |
| C1orf144 | | |
| CANT1 | prostate | Translocation with ETV4 |
| CARD11 | Lymphoma | |
| CARS | ALCL | Translocation with ALK |
| CASP8 | Breast | |
| CBFA2T1 | AML | Translocation with MLL or RUNX1 |
| CBFA2T3 | AML | Translocation with RUNX1 |
| CBFB | AML | Translocation with MYH11 |
| CBL | Lymphoma, leukemia | |
| CBL | AML, JMML, MDS | Translocation with MLL |
| CCND1 | CLL, B-ALL, breast | Translocation with IGH or FSTL3 |
| CCND2 | NHL,CLL, Retinoblastoma, mantle cell | Translocation with IGL |
| | lymphoma, T-cell acute lymphoblastic | |
| | leukemia (T-ALL), Burkitt lymphoma, | |
| | testicular germ cell tumor, ovarian | |
| | granulosa cell tumor, multiple myeloma | |
| CCND3 | MM, Retinoblastoma, mantle cell | Translocation with IGH |
| | lymphoma, anaplastic large cell | |
| | lymphoma, lymphoma (non-hodgkins), | |
| | B-cell lymphoma, laryngeal squamous | |
| | cell carcinoma, indolent lymphoma, | |
| | null cell adenoma | |
| CCNE1 | Breast cancer, ovarian cancer, bladder | |
| | cancer, retinoblastoma | |
| CD22 | NSCLC, breast | |
| CD74 | NSCLC | Translocation with ROS1 |
| CD79A | Diffuse large B-cell lymphoma (DLBCL) | |
| CD79B | DLBCL | |
| CDC73 | Parathyroid | |
| CDH11 | aneurysmal bone cysts, Gastric cancer, | Translocation with USP6 |
| | lobular carcinoma, squamous cell carcinoma, | |
| | invasive ductal carcinoma, invasive lobular | |
| | carcinoma | |
| CDH2 | Melanoma, malignant mesothelioma, pleural | |
| | mesothelioma, desmoplastic melanoma, lung | |
| | adenocarcinoma, endometrioid tumor, | |
| | mesothelioma, bladder cancer, esophageal | |
| | squamous cell carcinoma (ESCC) | |
| CDH20 | Breast cancer | |
| CDH5 | Granuloma, epithelioid sarcoma | |
| CDK12 | Ovarian | |
| CDK4 | Melanoma | |
| CDK6 | ALL | Translocation with MLLT10 |
| CDK8 | Colon cancer, lung cancer, rectal cancer, | |
| | acute lymphoblastic leukemia (ALL) | |
| CDKN1B | Breast | |
| CDKN2A | melanoma, pancreatic cancer, Li-Fraumeni | |
| | syndrome, lung cancer (e.g., non-small | |
| | cell lung cancer (NSCLC)), squamous cell | |
| | carcinoma, retinoblastoma, astrocytoma | |
| CDKN2B | Leukemia, retinoblastoma, laryngeal | |
| | squamous cell carcinoma | |
| CDKN2C | Thyroid carcinoma, pituitary adenoma, | |
| | oligodendroglioma, pancreatic endocrine | |
| | tumor, multiple myeloma, hepatoblastoma, | |
| | lymphoid tumor, multiple endocrine | |
| | neoplasia type 1, anaplastic | |
| | oligodendroglioma | |
| CDX2 | AML | Translocation with ETV6 |
| CEBPA | Leukemia (e.g., acute myeloid leukemia | |
| | (AML), acute myeloid leukemia (AML), | |
| | monoblastic leukemia), retinoblastoma | |
| CEP1 | MPD, NHL | Translocation with FGFR1 |
| CHCHD7 | salivary adenoma | Translocation with PLAG1 |
| CHEK1 | Leukemia, colon cancer | |
| CHEK2 | Breast cancer | |
| CHIC2 | AML | Translocation with ETV6 |
| CHN1 | extraskeletal myxoid chondrosarcoma | Translocation with TAF15 |
| CHUK | Colorectal | |
| CIC | soft tissue sarcoma | Translocation with DUX4 |
| CLTC | ALCL, renal | Translocation with ALK, TFE3 |
| CLTCL1 | ALCL | Translocation |
| CMKOR1 | lipoma | Translocation with HMGA2 |
| COL1A1 | dermatofibrosarcoma protuberans, | Translocation with PDGFB or USP6 |
| | aneurysmal bone cyst | |
| COX6C | uterine leiomyoma | Translocation with HMGA2 |
| CRBN | Upper aerodigestive tract | |
| CREB1 | clear cell sarcoma, angiomatoid fibrous | Translocation with EWSR1 |
| | histiocytoma | |
| CREB3L2 | fibromyxoid sarcoma | Translocation with FUS |
| CREBBP | Acute lymphoblastic leukemia (ALL), AML, | |
| | DLBCL, B-cell non-Hodgkin's lymphoma | |
| | (B-NHL) | |
| CREBBP | AL, AML | Translocation with MLL, MORF, or RUNXBP2 |
| CRKL | Leukemia, lymphoma | |
| CRLF2 | Leukemia | |
| CRTC3 | salivary gland mucoepidermoid | Translocation with MAML2 |
| CSF1R | NSCLC | |
| CTCF | Breast | |
| CTNNA1 | Breast | |
| CTNNB1 | Colorectal cancer, ovarian cancer, prostate | Codon 32, 33, 34, 37, 41, or 45 |
| | cancer, liver cancer (e.g., hepatoblastoma | |
| | (HB), hepatocellular carcinoma (HCC)), | |
| | pilomatrixoma, medulloblastoma, salivary | |
| | gland pleiomorphic adenomas | |
| CTNNB1 | colorectal, cvarian, hepatoblastoma, others, | Translocation with PLAG1 |
| | pleomorphic salivary adenoma | |
| CUL4A | Leukemia | |
| CUL4B | Leukemia | |
| CYP17A1 | Breast | |
| CYP1B1 | breast cancer | CYP1B1*3 |
| CYP2C19 | | CYP2C19*17 |
| CYP2C19 | | CYP2C19*17 |
| CYP2C8 | | 461delV |
| CYP2C8 | | K399R |
| CYP2D6 | | CYP2D6: 3183 G > A |
| CYP2D6 | | CYP2D6: 2988 G > A |
| CYP2D6 | | CYP2D6: 2850 C > T |
| CYP2D6 | | CYP2D6: 2613-2615 del AGA |
| CYP2D6 | | CYP2D6: 2549 del A |
| CYP2D6 | | CYP2D6: 1846 G > A |
| CYP2D6 | | CYP2D6: 1707 del T |
| CYP2D6 | | CYP2D6: 1659G > A |
| CYP2D6 | | CYP2D6: 1023 C > T |
| CYP2D6 | | CYP2D6: 100C > T |
| CYP3A4 | | CYP3A4*16B |
| CYP3A4 | | CYP3A4*1B |
| CYP3A5 | | |
| D10S170 | papillary thyroid, CML | Translocation with RET or PDGFRB |
| DAXX | Pancreatic neuroendocrine tumors | |
| DDIT3 | liposarcoma | Translocation with FUS |
| DDR2 | NSCLC | |
| DDX10 | AML* | Translocation with NUP98 |
| DDX5 | prostate | Translocation with ETV4 |
| DDX6 | B-NHL | Translocation with IGH |
| DEK | AML | Translocation with NUP214 |
| DIS3 | MM | |
| DNMT3A | Testicular germ cell tumor, lymphosarcoma, | |
| | hepatocellular carcinoma, salivary gland | |
| | tumor | |
| DOT1L | Leukemia | |
| DPYD | | DPYD*2A |
| DPYD | | DPYD*5 |
| DPYD | | 496A > G |
| DPYD | | DPYD*9A |
| DUX4 | soft tissue sarcoma | Translocation with CIC |
| EGFR | Lung cancer, squamous cell carcinoma, | Codon 719, 746-750, 768, 790 (e.g., T790M), 858 |
| | glioblastoma, glioma, colorectal cancer | (e.g., L858R), or 861 |
| EIF4A2 | NHL | Translocation with BCL6 |
| ELF4 | AML | Translocation with ERG |
| ELK4 | prostate | Translocation with SLC45A3 |
| ELKS | papillary thyroid | Translocation with RET |
| ELL | AL | Translocation with MLL |
| ELN | B-ALL | Translocation with PAX5 |
| EML4 | NSCLC | Translocation with ALK |
| EMSY | Breast | |
| EP300 | Colorectal, breast, pancreatic, AML, ALL, | |
| | DLBCL | |
| EP300 | colorectal, breast, pancreatic, AML | Translocation with MLL or RUNXBP2 |
| EP300 | colorectal, breast, pancreatic, AML | Translocation with MLL or RUNXBP2 |
| EPHA3 | Rhabdomyosarcoma, lymphoma, prostate | |
| | cancer, hepatocellular carcinoma, leukemia, | |
| | melanoma | |
| EPHA5 | Glioblastoma, breast cancer, astrocytoma, | |
| | Wilms' tumor, glioma | |
| EPHA6 | Breast cancer | |
| EPHA7 | Glioblastoma multiforme (GBM), colon | |
| | cancer, duodenal cancer, parathyroid | |
| | tumor, prostate cancer | |
| EPHB1 | Colorectal cancer, embryonal carcinoma, | |
| | gastric cancer, teratocarcinoma, | |
| | mucinous carcinoma | |
| EPHB4 | Head and neck squamous cell carcinoma | |
| | (HNSCC), brain cancer, endometrial cancer, | |
| | ovarian cancer | |
| EPHB6 | Neuroblastoma, melanoma, non-small cell | |
| | lung cancer (NSCLL) | |
| EPS15 | ALL | Translocation with MLL |
| ERBB2 | Gastric cancer, glioma, ovarian cancer, | Amplification |
| | lung cancer | |
| ERBB3 | Breast cancer, non-small cell lung cancer | |
| | (NSCLC), pancreatic cancer, invasive ductal | |
| | carcinoma, lung adenocarcinoma, | |
| | endometrioid carcinoma, pilocytic | |
| | astrocytoma | |
| ERBB4 | Breast cancer, medulloblastoma, cervical | |
| | squamous cell carcinoma, prostate cancer, | |
| | leukemia | |
| ERCC2 | Skin basal cell, skin squamous cell, | 2251A > C |
| | melanoma | |
| ERG | Ewing sarcoma, prostate, AML, Prostate | Translocation with EWSR1, TMPRSS2, ELF4, |
| | cancer, Ewing's sarcoma, leukemia, | FUS, or HERPUD1 |
| | prostate cancer | |
| ESR1 | Breast cancer, endometrial cancer, | |
| | endometrial adenocarcinoma, leiomyoma, | |
| | mammary ductal carcinoma | |
| ESR2 | | |
| ETV1 | Prostate cancer, breast cancer, Ewing's | Translocation with EWSR1, TMPRSS2, |
| | sarcoma, desmoplastic small round cell | SLC45A3, C15orf21, HNRNPA2B1, or ACSL3 |
| | tumor, myxoid liposarcoma, clear cell | |
| | sarcoma | |
| ETV4 | | |
| ETV4 | Breast cancer, ovarian cancer, squamous | Translocation with EWSR1, TMPRSS2, DDX5, |
| | cell carcinoma tongue, Ewing's | KLK2, or CANT1 |
| | sarcoma, Prostate carcinoma | |
| ETV5 | Ganglioglioma, brain tumor | |
| ETV5 | Prostate | Translocation with TMPRSS2 or SCL45A3 |
| ETV6 | leukemia, congenital fibrosarcoma, multiple | Translocation with NTRK3, RUNX1, PDGFRB, |
| | leukemia and lymphoma, secretory breast, | ABL1, MN1, ABL2, FACL6, CHIC2, ARNT, |
| | secretory carcinoma, MDS, ALL, | JAK2, EVI1, CDX2, STL, HLXB9, MDS2, PER1, |
| | myelodysplastic syndrome | SYK, TTL, FGFR3, or PAX5 |
| EVI1 | AML, CML | Translocation with RUNX1, ETV6, PRDM16, or |
| | | RPN1 |
| EWSR1 | Ewing sarcoma, desmoplastic small round | Translocation with FLI1, ERG, ZNF278, NR4A3, |
| | cell tumor, ALL, clear cell sarcoma, | FEV, ATF1, ETV1, ETV4, WT1, ZNF384, |
| | sarcoma, myoepithelioma, extraskeletal | CREB1, POU5F1, or PBX1 |
| | myxoid chondrosarcoma, myxoid | |
| | liposarcoma, angiomatoid fibrous | |
| | histiocytoma | |
| EZH2 | Prostate cancer, gallbladder adenocarci- | |
| | noma, breast cancer, bladder cancer, | |
| | gastric cancer, Ewing's sarcoma | |
| FACL6 | AML, AEL | Translocation with ETV6 |
| FAM46C | MM | |
| FANCA | Leukemia | |
| FANCC | AML, leukemia | |
| FANCD2 | AML, leukemia | |
| FANCE | AML, leukemia | |
| FANCG | AML, leukemia | |
| FANCL | AML, leukemia | |
| FBXW7 | Colorectal cancer, endometrial cancer, T-cell | |
| | acute lymphoblastic leukemia (T-ALL) | |
| FCGR2B | ALL | Translocation |
| FCGR3A | | V158F |
| FEV | Ewing sarcoma | Translocation with EWSR1 or FUS |
| FGF1 | Breast | |
| FGF10 | Breast | |
| FGF12 | Breast | |
| FGF14 | Breast | |
| FGF19 | Breast | |
| FGF23 | Breast | |
| FGF3 | Breast | |
| FGF4 | Breast | |
| FGF6 | Breast | |
| FGF7 | Breast | |
| FGFR1 | | |
| FGFR1 | MPD, NHL, Leukemia, lymphoma | Translocation with BCR, FOP, ZNF198, or CEP1 |
| FGFR1OP | MPD, NHL | Translocation with FGFR1 |
| FGFR2 | Breast cancer, prostate cancer | |
| FGFR3 | bladder, MM, T-cell lymphoma, cervical | Translocation with IGH or ETV6 |
| | cancer, | |
| FGFR4 | Pituitary tumor, prostate cancer, lung cancer, | |
| | astrocytoma, rhabdomyosarcoma, pituitary | |
| | adenoma, fibroadenoma | |
| FGFR4 | | GLY388ARG |
| FIP1L1 | idiopathic hypereosinophilic syndrome | Translocation with PDGFRA |
| FLI1 | Ewing sarcoma, Breast cancer, prostate | Translocation with EWSR1 |
| | cancer | |
| FLT3 | Leukemia (e.g., acute myeloid leukemia | Codon 835 |
| | (AML), acute promyelocytic leukemia, acute | |
| | lymphoblastic leukemia) | |
| FLT4 | Lung cancer, Kaposi's sarcoma, gastric | |
| | cancer, lymphangioma, squamous cell | |
| | carcinoma | |
| FNBP1 | AML | Translocation with MLL |
| FOXL2 | Granulosa-cell tumour of the ovary | Codon 134 |
| FOXO1A | alveolar rhabdomyosarcomas | Translocation with PAX3 |
| FOXO3A | AL | Translocation with MLL |
| FOXP1 | ALL | Translocation with PAX5 |
| FOXP4 | Lymphoma, brain tumor | |
| FSTL3 | B-CLL | Translocation with CCND1 |
| FUS | liposarcoma, AML, Ewing sarcoma, | Translocation with DDIT3, ERG, FEV, ATF1, or |
| | angiomatoid fibrous histiocytoma, | CREB3L2 |
| | fibromyxoid sarcoma | |
| FVT1 | B-NHL | Translocation with IGK |
| GAS7 | AML* | Translocation with MLL |
| GATA1 | Megakaryoblastic leukemia of Downs | |
| | Syndrome | |
| GATA2 | AML, Chronic Myeloid Leukemia (CML, | |
| | blast transformation) | |
| GATA3 | Breast | |
| GMPS | AML | Translocation with MLL |
| GNA11 | Breast cancer | |
| GNAQ | Uveal melanoma | |
| GNAS | Pituitary adenoma | |
| GOLGA5 | papillary thyroid | Translocation with RET |
| GPHN | AL | Translocation with MLL |
| GPR124 | Colon cancer | |
| GRAF | AML, MDS | Translocation with MLL |
| GRIN2A | Malignant melanoma | |
| GSK3B | NSCLC | |
| GSTP1 | | I105V |
| GSTP1 | | A114V |
| GUCY1A2 | Breast cancer | |
| HCMOGT-1 | JMML | Translocation with PDGFRB |
| HEAB | AML | Translocation with MLL |
| HEI10 | uterine leiomyoma | Translocation with HMGA2 |
| HERPUD1 | prostate | Translocation with ERG |
| HGF | MM | |
| HIP1 | CMML | Translocation with PDGFRB |
| HIST1H4I | NHL | Translocation with BCL6 |
| HLA-A | MM | |
| HLF | ALL | Translocation with TCF3 |
| HLXB9 | AML | Translocation with ETV6 |
| HMGA1 | microfollicular thyroid adenoma, various | Translocation |
| | benign mesenchymal tumors | |
| HMGA2 | lipoma | Translocation with LHFP, RAD51L1, LPP, |
| | | HEI10, COX6C, CMKOR1, or NFIB |
| HNRNPA2B1 | prostate | Translocation with ETV 1 |
| HOOK3 | papillary thyroid | Translocation with RET |
| HOXA11 | CML | Translocation with NUP98 |
| HOXA13 | AML | Translocation with NUP98 |
| HOXA3 | Breast cancer | |
| HOXA9 | AML* | Translocation with NUP98 or MSI2 |
| HOXC11 | AML | Translocation with NUP98 |
| HOXC13 | AML | Translocation with NUP98 |
| HOXD11 | AML | Translocation with NUP98 |
| HOXD13 | AML* | Translocation with NUP98 |
| HRAS | Hurthle cell thyroid carcinoma, bladder | Codon 12, 13, 61 |
| | cancer, melanoma, colorectal cancer | |
| HSP90AA1 | Lymphoma, myeloma | |
| HSPCA | NHL | Translocation with BCL6 |
| HSPCB | NHL | Translocation with BCL6 |
| IDH1 | Glioblastoma multiforme (GBM) | |
| IDH2 | Glioblastoma multiforme (GBM) | |
| IGF1 | Breast | |
| IGF1R | Ewing's sarcoma, breast cancer, uveal | |
| | melanoma, adrenocortical carcinoma, | |
| | pancreatic cancer | |
| IGF2 | Breast | |
| IGF2R | Gastrointestinal tumor, liver cancer | |
| IGH | MM, Burkitt lymphoma, NHL, CLL, B-ALL, | Translocation with MYC, FGFR3, PAX5, IRTA1, |
| | MALT, MLCLS | IRF4, CCND1, BCL9, BCL8, BCL6, BCL2, |
| | | BCL3, BCL10, BCL11A. LHX4, DDX6, NFKB2, |
| | | PAFAH1B2, or PCSK7 |
| IGK | Burkitt lymphoma, B-NHL | Translocation with MYC or FVT1 |
| IGL | Burkitt lymphoma | Translocation with BCL9, MYC, or CCND2 |
| IKBKE | Breast cancer | |
| IKZF1 | Lymphoma, leukemia | |
| IL2 | intestinal T-cell lymphoma | Translocation with TNFRSF17 |
| IL21R | NHL | Translocation with BCL6 |
| IL7R | T-cell acute lymphoblastic leukemia (T-ALL) | |
| INHBA | Erythroleukemia, barrett metaplasia, | |
| | esophageal adenocarcinoma, granulosa cell | |
| | tumor, sex cord-stromal tumor, lung | |
| | adenocarcinoma, pheochromocytoma, | |
| | krukenberg tumor, ovarian cancer | |
| INSR | NSCLC, glioblastoma, gastric | |
| IRF4 | Multiple myeloma (MM) | |
| IRF4 | MM | Translocation with IGH |
| IRS2 | Hyperinsulinemia, uterine leiomyosarcoma | |
| IRTA1 | B-NHL | Translocation with IGH |
| ITK | peripheral T-cell lymphoma | Translocation with SYK |
| ITPA | | |
| JAK1 | Leukemia, ovarian cancer, breast | |
| | cancer | |
| JAK2 | Leukemia (e.g., chronic lymphoblastic | Codon 617 |
| | leukemia (CLL), acute lymphoblastic | |
| | leukemia (ALL), chronic myelogenous | |
| | leukemia (CML), acute myelogenous | |
| | leukemia (AML)) | |
| JAK2 | ALL, AML, MPD, CML | Translocation with ETV6, PCM1, or BCR |
| JAK3 | Acute lymphoblastic leukemia (ALL) | |
| JAZF1 | endometrial stromal tumours | Translocation with SUZ12 |
| JUN | Skin cancer, leukemia | |
| KDM4C | Ovarian, breast | |
| KDM5A | AML | Translocation with NUP98 |
| KDM6A | Renal, oesophageal squamous cell | |
| | carcinoma (SCC), MM | |
| KDR | Non-small cell lung cancer (NSCLC), | |
| | angiosarcoma | |
| KEAP1 | NSCLC | |
| KIT | Gastrointestinal stromal tumor (GIST), | Codon 816 |
| | testicular tumor, leukemia (e.g., acute | |
| | myeloid leukemia (AML)), mast cell tumor, | |
| | mesenchymal tumor, adenoid cystic | |
| | carcinoma, lung cancer (e.g., small cell lung | |
| | cancer), lymphoma e.g., Burkitt lymphoma) | |
| KLHL6 | Chronic lymphocytic leukaemia (CLL) | |
| KLK2 | prostate | Translocation with ETV4 |
| KRAS | Leukemia (e.g., acute myelogenous | Codon 12, 13 (e.g., G13D), or 61 |
| | leukemia (AML), juvenile myelomonocytic | |
| | leukemia (JMML)), colorectal cancer, lung | |
| | cancer | |
| KTN1 | papillary thyroid | Translocation with RET |
| LAF4 | ALL, T-ALL | Translocation with MLL or RUNX1 |
| LASP1 | AML | Translocation with MLL |
| LCK | T-ALL | Translocation with TRB |
| LCP1 | NHL | Translocation with BCL6 |
| LCX | AML | Translocation with MLL |
| LHFP | lipoma | Translocation with HMGA2 |
| LIFR | salivary adenoma | Translocation with PLAG1 |
| LMO1 | T-ALL, neuroblastoma | Translocation with TRD |
| LMO2 | T-ALL | Translocation with TRD |
| LPP | lipoma, leukemia | Translocation with HMGA2, MLL, or C12orf9 |
| LRP1B | Lung cancer, gastric cancer, esophageal | |
| | cancer | |
| LRP2 | | |
| LRP6 | NSCLC, malignant melanoma | |
| LRRK2 | Ovarian, NSCLC | |
| LTK | Lymphoma, breast cancer | |
| LYL1 | T-ALL | Translocation with TRB |
| MAF | MM | Translocation with IGH |
| MAFB | MM | Translocation with IGH |
| MAGED1 | MM | |
| MALT1 | MALT | Translocation with BIRC3 |
| MAML2 | salivary gland mucoepidermoid | Translocation with MECT1 or CRTC3 |
| MAN1B1 | | |
| MAP2K1 | Prostate cancer, gastric cancer | |
| MAP2K2 | Pancreatic cancer, intestinal tumor | |
| MAP2K4 | Pancreatic cancer, breast cancer, | |
| | colorectal cancer | |
| MAP3K1 | Breast | |
| MAP3K13 | Breast | |
| MCL1 | Multiple myeloma, leukemia, lymphoma | |
| MDM2 | Sarcoma, glioma, colorectal cancer | |
| MDM4 | Glioblastoma multiforme (GBM), bladder | |
| | cancer, retinoblastoma | |
| MDS1 | MDS, AML | Translocation with RUNX1 |
| MDS2 | MDS | Translocation with ETV6 |
| MECT1 | salivary gland mucoepidermoid | Translocation with MAML2 |
| MEN1 | Parathyroid tumor | |
| MET | Gastric cancer, hepatocellular carcinoma | |
| | (HCC), hereditary papillary renal carcinoma | |
| | (HPRC), lung cancer (e.g., non-small cell | |
| | lung cancer), papillary thyroid carcinoma, | |
| | glioma, esophageal adenocarcinoma, | |
| | osteosarcoma, endometrial cancer, squamous | |
| | cell carcinoma, melanoma, breast cancer | |
| MHC2TA | NHL | Translocation with BCL6 |
| MITF | Melanoma | |
| MKL1 | acute megakaryocytic leukemia | Translocation with RBM15 |
| MLF1 | AML | Translocation with NPM1 |
| MLH1 | Colorectal cancer, endometrial cancer, | |
| | ovarian cancer, CNS cancer | |
| MLL | Leukemia (e.g., acute lymphoblastic | |
| | leukemia (ALL), acute myeloid leukemia | |
| | (AML) | |
| MLL | AML, ALL | Translocation with MLL, MLLT1, MLLT2, |
| | | MLLT3, MLLT4, MLLT7, MLLT10, MLLT6, |
| | | ELL, EPS15, AF1Q, CREBBP, SH3GL1, FNBP1, |
| | | PNUTL1, MSF, GPHN, GMPS, SSH3BP1, |
| | | ARHGEF12, GAS7, FOXO3A, LAF4, LCX, |
| | | SEPT6, LPP, CBFA2T1, GRAF, EP300, PICALM, |
| | | or HEAB |
| MLL2 | Medulloblastoma, renal | |
| MLLT1 | AL | Translocation with MLL |
| MLLT10 | AL | Translocation with MLL, PICALM, or CDK6 |
| MLLT2 | AL | Translocation with MLL |
| MLLT3 | ALL | Translocation with MLL |
| MLLT4 | AL | Translocation with MLL |
| MLLT6 | AL | Translocation with MLL |
| MLLT7 | AL | Translocation with MLL |
| MLST8 | Breast | |
| MN1 | AML, meningioma | Translocation with ETV6 |
| MN1 | AML, meningioma | Translocation with ETV6 |
| MPL | Myeloproliferative disorder (MPD) | |
| MRE11A | Breast cancer, lymphoma | |
| MSF | AML | Translocation with MLL |
| MSH2 | Colorectal cancer, endometrial cancer, | Biallelic inactivation |
| | ovarian cancer | |
| MSH6 | Colorectal cancer | |
| MSI2 | CML | Translocation with HOXA9 |
| MSN | ALCL | Translocation with ALK |
| MTCP1 | T cell prolymphocytic leukemia | Translocation with TRA |
| MTHFR | | 677C > T |
| MTOR | Lymphoma lung cancer, renal cancer, clear | |
| | cell carcinoma, glioma | |
| MUC1 | B-NHL | Translocation with IGH |
| MUTYH | Colorectal cancer | |
| MYB | adenoid cystic carcinoma | Translocation with NFIB |
| MYC | chronic lymphocytic leukemia (CLL), | Amplification |
| | Burkitt lymphoma, plasmacytoma, breast | |
| | cancer | |
| MYC | Burkitt lymphoma, amplified in other | Translocation with IGK, BCL5, BCL7A, BTG1, |
| | cancers, B-CLL | TRA, or IGH |
| MYCL1 | Small cell lung cancer (SCLC) | |
| MYCN | Neuroblastoma | |
| MYD88 | Activated B cell-like-DLBCL (ABC-DLBCL) | |
| MYH11 | AML | Translocation with CBFB |
| MYH9 | ALCL | Translocation with ALK |
| MYST3 | Breast | |
| MYST4 | AML | Translocation with CREBBP |
| NACA | NHL | Translocation with BCL6 |
| NCOA1 | alveolar rhadomyosarcoma | Translocation with PAX3 |
| NCOA2 | AML | Translocation with RUNXBP2 |
| NCOA4 | papillary thyroid | Translocation with RET |
| NCOR1 | Breast | |
| NF1 | Leukemia (e.g., juvenile myelomonocytic | |
| | leukemia (JMML)), neurofibroma, | |
| NF2 | Meningioma, acoustic neuroma, renal | |
| | cancer | |
| NFE2L2 | NSCLC, head and neck squamous cell | |
| | carcinoma (HNSCC) | |
| NFIB | adenoid cystic carcinoma, lipoma | Translocation with MYB or HGMA2 |
| NFKB1 | Breast | |
| NFKB2 | B-NHL | Translocation with IGH |
| NFKBIA | Breast | |
| NIN | MPD | Translocation with PDGFRB |
| NKX2-1 | Lung cancer, thyroid cancer, | |
| | adenocarcinoma | |
| NONO | papillary renal cancer | Translocation with TFE3 |
| NOTCH1 | Squamous cell carcinoma, leukemia (e.g., | Codon 1575 or 1601 |
| | acute lymphoblastic leukemia (ALL)), | |
| | medullary thyroid carcinoma, lymphoma | |
| | (e.g., thymic lymphoma, T-cell lymphoma) | |
| NOTCH1 | T-ALL | Translocation with TRB |
| NOTCH2 | Marginal zone lymphoma, DLBCL | |
| NOTCH3 | NSCLC, breast | |
| NOTCH4 | NSCLC, breast | |
| NPM1 | Lymphoma (e.g., non-Hodgkin lymphoma, | |
| | anaplastic large cell lymphoma, anaplastic | |
| | lymphoma), leukemia (e.g., acute | |
| | promyelocytic leukemia, acute myelogenous | |
| | leukemia (AML)) | |
| NPM1 | NHL, APL, AML | Translocation with ALK, RARA, or MLF1 |
| NQO1 | breast cancer | NQO1*2 |
| NR4A3 | extraskeletal myxoid chondrosarcoma | Translocation with EWSR1 |
| NRAS | Leukemia (e.g., juvenile myelomonocytic | Codon 12, 13, 61 (e.g., Q61K or Q61H) |
| | leukemia (JMML), acute myeloid leukemia | |
| | (AML), acute lymphoblastic leukemia), | |
| | melanoma, colon cancer | |
| NRP2 | | |
| NSD1 | AML | |
| NSD1 | AML | Translocation with NUP98 |
| NTRK1 | papillary thyroid | Translocation with TPM3, TPR, or TFG |
| NTRK2 | Renal, NSCLC | |
| NTRK3 | Congenital fibrosarcoma, secretory | |
| | breast cancer | |
| NTRK3 | congenital fibrosarcoma, Secretory breast | Translocation with ETV6 |
| NUMA1 | APL | Translocation with RARA |
| NUP214 | AML, T-ALL | Translocation with DEK, SET, or ABL1 |
| NUP93 | Breast | |
| NUP98 | AML | Translocation with HOXA9, NSD1, WHSC1L1, |
| | | DDX10, TOP1, HOXD13, PMX1, HOXA13, |
| | | HOXD11, HOXA11, RAP1GDS1, or HOXC11 |
| NUT | lethal midline carcinoma of young people | Translocation with BRD4 or BRD3 |
| OLIG2 | T-ALL | Translocation with TRA |
| OMD | aneurysmal bone cysts | Translocation with USP6 |
| PAFAH1B2 | MLCLS | Translocation with IGH |
| PAK3 | Lung cancer | |
| PAK7 | NSCLC, malignant melanoma | |
| PALB2 | Wilms tumor, medulloblastoma, AML, | |
| | breast | |
| PAX3 | alveolar rhabdomyosarcoma | Translocation with FOXO1A or NCOA1 |
| PAX5 | Non-Hodgkin Lymphoma (NHL), acute | |
| | lymphoblastic leukemia (ALL, e.g., B-cell | |
| | ALL) | |
| PAX5 | NHL, ALL, B-ALL | Translocation with IGH, ETV6, PML, FOXP1, |
| | | ZNF521, or ELN |
| PAX7 | alveolar rhabdomyosarcoma | Translocation with FOXO1A |
| PAX8 | follicular thyroid | Translocation with PPARG |
| PBRM1 | Clear cell renal carcinoma, breast | |
| PBX1 | pre B-ALL, myoepithelioma | Translocation with TCF3 or EWSR1 |
| PCM1 | papillary thyroid, CML, MPD | Translocation with RET or JAK2 |
| PCSK7 | MLCLS | Translocation with IGH |
| PDE4DIP | MPD | Translocation with PDGFRB |
| PDGFB | DFSP | Translocation with COL1A1 |
| PDGFRA | Gastrointestinal stromal tumor (GIST), | |
| | leukemia (e.g., chronic eosinophilic | |
| | leukemia (CEL), acute lymphocytic | |
| | leukemia (ALL)), mesenchymal tumor | |
| PDGFRA | GIST, idiopathic hypereosinophilic | Translocation with FIP1L1 |
| | syndrome | |
| PDGFRB | Myeloproliferative disorder (MPD), acute | |
| | myeloid leukemia (AML), chronic myeloid | |
| | leukemia (CML), chronic myelomonocytic | |
| | leukemia (CMML) | |
| PDGFRB | MPD, AML, CMML, CML | Translocation with ETV6, TRIP11, HIP1, |
| | | RAB5EP, H4, NIN, HCMOGT-1, or PDE4DIP |
| PDK1 | NSCLC | |
| PER1 | AML, CMML | Translocation with ETV6 |
| PHLPP2 | Ovarian, glioblastoma, NSCLC | |
| PHOX2B | Neuroblastoma | |
| PICALM | TALL, AML, | Translocation with MLLT10 or MLL |
| PIK3C2G | NSCLC | |
| PIK3C3 | NSCLC | |
| PIK3CA | Colorectal cancer, breast cancer, ovarian | Codon 88, 542, 545, 546, 1047 (e.g., H1047R), or |
| | cancer, hepatocellular carcinoma, head and | 1049 |
| | neck squamous cell carcinoma (HNSCC), | |
| | anaplastic thyroid carcinoma, endometrial | |
| | cancer, gallbladder adenocarcinoma, | |
| | glioblastoma | |
| PIK3CG | NSCLC | |
| PIK3R1 | Glioblastoma, ovarian cancer, colorectal | |
| | cancer | |
| PIK3R2 | NSCLC | |
| PIM1 | NHL | Translocation with BCL6 |
| PKHD1 | Pancreatic cancer | |
| PLAG1 | salivary adenoma | Translocation with TCEA1, LIFR, CTNNB1, or |
| | | CHCHD7 |
| PLCG1 | Head and neck cancer, leukemia | |
| PML | APL, ALL | Translocation with RARA or PAX5 |
| PMX1 | AML | Translocation with NUP98 |
| PNRC1 | MM | |
| PNUTL1 | AML | Translocation with MLL |
| POU2AF1 | NHL | Translocation with BCL6 |
| POU5F1 | sarcoma | Translocation with EWSR1 |
| PPARG | follicular thyroid | Translocation with PAX8 |
| PRCC | papillary renal | Translocation with TFE3 |
| PRDM1 | DLBCL | |
| PRDM16 | MDS, AML | Translocation with EVI1 |
| PRKAR1A | Adrenal gland, thyroid | |
| PRKAR1A | papillary thyroid | Translocation with RET |
| PRKDC | Glioma, glioblastoma, gastric cancer, | |
| | ovarian cancer | |
| PRO1073 | renal cell carcinoma (childhood epithelioid) | Translocation with TFEB |
| PRSS8 | Breast | |
| PSIP2 | AML | Translocation with NUP98 |
| PTCH1 | Skin basal cell, medulloblastoma | |
| PTCH2 | Malignant melanoma | |
| PTEN | Head and neck squamous cell carcinomas | Codon 130, 173, 233, or 267; biallelic inactivation |
| | (HNSCC), endometrial cancer, glioma, | |
| | prostate cancer, glioblastoma, colon | |
| | cancer, breast cancer | |
| PTK2 | NSCLC, glioblastoma | |
| PTK2B | NSCLC, breast | |
| PTPN11 | Juvenile myelomonocytic leukemia | |
| | (JMML), acute myeloid leukemia (AML), | |
| | myelodysplastic syndromes (MDS) | |
| PTPRD | Lung cancer, cutaneous squamous cell | |
| | carcinoma, glioblastoma, neuroblastoma | |
| RAB5EP | CMML | Translocation with PDGFRB |
| RAD50 | Breast | |
| RAD51 | Breast | |
| RAD51L1 | lipoma, uterine leiomyoma | Translocation with HMGA2 |
| RAFI | pilocytic astrocytoma | Translocation with SRGAP3 |
| RANBP17 | ALL | Translocation with TRD |
| RAP1GDS1 | T-ALL | Translocation with NUP98 |
| RARA | Leukemia | |
| RARA | APL | Translocation with PML, ZNF145, TIF1, NUMA1, or |
| | | NPM1 |
| RBI | Retinoblastoma, bladder cancer, | |
| | osteosarcoma, lung cancer (e.g., small | |
| | cell lung cancer, non-small cell lung | |
| | cancer), leukemia (e.g., acute | |
| | lymphoblastic leukemia (ALL)) | |
| RBM15 | acute megakaryocytic leukemia | Translocation with MKL1 |
| REL | Hodgkin Lymphoma | |
| RET | Colorectal cancer, medullary thyroid | Codon 918 |
| | carcinoma, multiple neoplasia type 2B, | |
| | pheochromocytoma, multiple neoplasia type | |
| | 2A, thyroid papillary carcinoma, thryoid | |
| | cancer, retinoblastoma | |
| RET | medullary thyroid, papillary thyroid, | Translocation with H4, PRKAR1A, NCOA4, |
| | pheochromocytoma | PCM1, GOLGA5, TRIM33, KTN1, TRIM27, or |
| | | HOOK3 |
| RHEB | NSCLC, colorectal | |
| RICTOR | Colon cancer, lymphoma, glioma, breast | |
| | cancer | |
| ROCK1 | Breast | |
| ROS1 | glioblastoma, NSCLC | Translocation with GOPC or ROS1 |
| RPL22 | AML, CML | Translocation with RUNX1 |
| RPN1 | AML | Translocation with EVI1 |
| RPTOR | Breast cancer, prostate cancer | |
| RUNX1 | Acute myeloid leukemia (AML), pre-B-cell | |
| | acute lymphoblastic leukemia (preB-ALL), | |
| | T-cell acute lymphoblastic leukemia | |
| | (T- ALL) | |
| RUNX1 | AML, preB- ALL, T-ALL | Translocation with RPL22, MDS1, EVI1, |
| | | CBFA2T3, CBFA2T1, ETV6, or LAF4 |
| RUNXBP2 | AML | Translocation with CREBBP, NCOA2, or EP300 |
| RUNXT1 | NSCLC, colorectal | |
| SEPT6 | AML | Translocation with MLL |
| SET | AML | Translocation with NUP214 |
| SETD2 | Clear cell renal carcinoma | |
| SF3B1 | MDS, CML, ALL, pancreatic, breast | |
| SFPQ | papillary renal cell | Translocation with TFE3 |
| SFRS3 | follicular lymphoma | Translocation with BCL6 |
| SH2B3 | Myelodysplastic syndrome (MDS) | |
| SH3GL1 | AL | Translocation with MLL |
| SIL | T-ALL | Translocation with TAL1 |
| SLC19A1 | | |
| SLC22A2 | | Ala270Ser |
| SLC45A3 | prostate | Translocation with ETV1, ETV5, ELK4, or ERG |
| SLCO1B3 | | |
| SMAD2 | esophageal squamous cell carcinoma | |
| | (ESCC) | |
| SMAD3 | Skin cancer, choriocarcinoma | |
| SMAD4 | Pancreatic cancer, colon cancer | |
| SMARCA4 | Non-small cell lung cancer (NSCLC) | |
| SMARCB1 | Malignant rhabdoid | |
| SMO | Skin basal cell cancer | |
| SOCS1 | DLBCL | |
| SOD2 | breast cancer | V16A |
| SOX10 | Oligodendroglioma | |
| SOX2 | Embryonal carcinoma, germ cell tumor | |
| SPEN | Adenoid cystic carcinoma | |
| SPOP | Malignant melanoma | |
| SRC | Sarcoma, colon cancer, breast cancer | |
| SRGAP3 | pilocytic astrocytoma | Translocation with RAF1 |
| SS18 | synovial sarcoma | Translocation with SSX1 or SSX2 |
| SS18L1 | synovial sarcoma | Translocation with SSX1 |
| SSH3BP1 | AML | Translocation with MLL |
| SSX1 | synovial sarcoma | Translocation with SS18 |
| SSX2 | synovial sarcoma | Translocation with SS18 |
| SSX4 | synovial sarcoma | Translocation with SS18 |
| STAG2 | Glioblastoma | |
| STAT3 | Breast | |
| STAT4 | Breast | |
| STK11 | Non-small cell lung cancer (NSCLC), | |
| | pancreatic cancer | |
| STK12 | PNET, NSCLC | |
| STL | B-ALL | Translocation with ETV6 |
| SUFU | Medulloblastoma | |
| SULT1A1 | | |
| SUZ12 | endometrial stromal tumours | Translocation with JAZF1 |
| SYK | MDS, peripheral T-cell lymphoma | Translocation with ETV6, ITK |
| TAF15 | extraskeletal myxoid chondrosarcomas, ALL | Translocation with TEC, CHN1, or ZNF384 |
| TAL1 | lymphoblastic leukemia/biphasic | Translocation with TRD, or SIL |
| TAL2 | T-ALL | Translocation with TRB |
| TBX22 | Breast cancer | |
| TBX23 | Breast | |
| TBX3 | Breast | |
| TCEA1 | salivary adenoma | Translocation with PLAG1 |
| TCF12 | extraskeletal myxoid chondrosarcoma | Translocation with TEC |
| TCF3 | pre B-ALL | Translocation with PBX1, HLF, or TFPT |
| TCL1A | T-CLL | Translocation with TRA |
| TCL6 | T-ALL | Translocation with TRA |
| TET2 | Myelodysplastic syndromes (MDS) | |
| TFE3 | papillary renal, alveolar soft part sarcoma, | Translocation with SFPQ, ASPSCR1, PRCC, |
| | renal | NONO, or CLTC |
| TFEB | renal (childhood epithelioid) | Translocation with ALPHA |
| TFG | papillary thyroid, ALCL, NSCLC | Translocation with NTRK1 or ALK |
| TFPT | pre-B ALL | Translocation with TCF3 |
| TFRC | NHL | Translocation with BCL6 |
| TGFBR2 | Lung cancer, gastric cancer, colon cancer | |
| THRAP3 | aneurysmal bone cysts | Translocation with USP6 |
| TIF1 | APL | Translocation with RARA |
| TLX1 | T-ALL | Translocation with TRB or TRD |
| TLX3 | T-ALL | Translocation with BCL11B |
| TMPRSS2 | prostate | Translocation with ERG, ETV1, ETV4, or ETV5 |
| TMPT | | TPMT*3B |
| TNFAIP3 | Marginal zone B-cell lymphomas, Hodgkin's | |
| | lymphoma, primary mediastinal B cell | |
| | lymphoma | |
| TNFRSF14 | Follicular lymphoma | |
| TNFRSF17 | Intestinal T-cell lymphoma | |
| TNFRSF17 | intestinal T-cell lymphoma | Translocation with IL2 |
| TNKS | NSCLC | |
| TNKS2 | Melanoma, breast | |
| TOP1 | AML | Translocation with NUP98 |
| TP53 | TP53 is frequently mutated or inactivated | Codon 175, 245, 248, 273, or 306 |
| | in about 60% of cancers, e.g., esophageal | |
| | squamous cell carcinoma, Li-Fraumeni | |
| | syndrome, head and neck squamous cell | |
| | carcinomas (HNSCC), lung cancer, | |
| | hereditary adrenocortical carcinoma, | |
| | astrocytoma, squamous cell carcinoma, | |
| | bladder cancer, colorectal cancer, | |
| | glioblastoma, retinoblastoma | |
| TPM3 | papillary thyroid, ALCL | Translocation with NTRK1 or ALK |
| TPM4 | ALCL | Translocation with ALK |
| TPMT | | |
| TPR | papillary thyroid | Translocation with NTRK1 |
| TRA | T-ALL | Translocation with ATL, OLIG2, MYC, TCL1A, |
| | | TCL6, MTCP1, or TCL6 |
| TRB | T-ALL | Translocation with HOX11, LCK, NOTCH1, |
| | | TAL2, or LYL1 |
| TRD | T-cell leukemia | Translocation with TAL1, HOX11, TLX1, LMO1, |
| | | LMO2, or RANBP17 |
| TRIM27 | papillary thyroid | Translocation with RET |
| TRIM33 | papillary thyroid | Translocation with RET |
| TRIP11 | AML | Translocation with PDGFRB |
| TRRAP | Colorectal, glioblastoma | |
| TSC1 | Hamartoma, renal cell cancer | |
| TSC2 | Hamartoma, renal cell cancer | |
| TTL | ALL | Translocation with ETV6 |
| TYK2 | NSCLC, breast | |
| TYMS | | 28 bp tandem repeat |
| TYMS | | 6 bp deletion |
| UGT1A1 | | |
| UMPS | | Gly213Ala |
| USP6 | aneurysmal bone cysts | Translocation with COL1A1, CDH11, ZNF9, or OMD |
| USP9X | Leukemia | |
| VHL | Renal cancer, hemangioma, | Biallelic inactivation |
| | pheochromocytoma | |
| WHSC1 | MM | Translocation with IGH |
| WHSC1L1 | AML | Translocation with NUP98 |
| WT1 | Wilms' tumor, desmoplastic small round | |
| | cell tumor | |
| XBP1 | MM | |
| XPO1 | Chronic lymphocytic leukaemia (CLL) | |
| ZNF145 | APL | Translocation with RARA |
| ZNF198 | MPD, NHL | Translocation with FGFR1 |
| ZNF217 | Breast | |
| ZNF278 | Ewing sarcoma | Translocation with EWSR1 |
| ZNF331 | follicular thyroid adenoma | Translocation |
| ZNF384 | ALL | Translocation with EWSR1, or TAF15 |
| ZNF521 | ALL | Translocation with PAX5 |
| ZNF703 | Breast | |
| ZNF9 | aneurysmal bone cysts | Translocation with USP6 |
| ZNFN1A1 | ALL, DLBL | Translocation with BCL6 |

TABLE 9
Additional exogenous polypeptide sequences

| Agent | Sequence |
|---|---|
| 4-1BBL | ACPWAVSGARASPGSAASPRLREGPELSPDDPAGLLDLRQG |
| | MFAQLVAQNVLLIDGPLSWYSDPGLAGVSLTGGLSYKEDTK |
| | ELVVAKAGVYYVFFQLELRRVVAGEGSGSVSLALHLQPLRS |
| | AAGAAALALTVDLPPASSEARNSAFGFQGRLLHLSAGQRLG |
| | VHLHTEARARHAWQLTQGATVLGLFRVTPEIPAGLPSPRSE |
| | (SEQ ID NO: 80) |
| Anti-PD- | VQLVESGGGLVQPGGSLRLSCAASGFTFSDSWIHWVRQAPG |
| L1 scFv | KGLEWVAWISPYGGSTYYADSVKGRFTISADTSKNTAYLQM |
| | NSLRAEDTAVYYCARRHWPGGFDYWGQGTLVTVSSGGGGSG |
| | GGGSGGGGSIQMTQSPSSLSASVGDRVTITCRASQDVSTAV |
| | AWYQQKPGKAPKLLIYSASFLYSGVPSRFSGSGSGTDFTLT |
| | ISSLQPEDFATYYCQQYLYHPATFGQGTKVEIK |
| | (SEQ ID NO: 81) |

### Examples

### Example 1: Erythroid Cells Expressing an Exogenous Polypeptide Comprising a Binding Agent to a Tumor Antigen Bind to and Promote Apoptosis in Cells Expressing the Tumor Antigen, and Penetrate Solid Tumors

Enucleated erythroid cells were produced which express on their surface a fusion protein comprising, from N-to-C terminus, a rituximab CD20 scFv domain, an HA epitope tag, and full-length GPA (including the extracellular, transmembrane, and intracellular domains) (RCT-antiCD20). Control enucleated erythroid cells were produced which express on their surface just the HA epitope tag fused to the N terminus of full-length GPA (RCT-HA-GPA). Method for producing erythroid cells expressing an exogenous protein are described, e.g., in WO2015/073587.

The RCT-antiCD20 bound to numerous CD20-expressing cell lines, as shown by flow cytometry (see Table 6). Amount of binding correlated with the levels of CD20 of the cell lines (data not shown).

Binding of the RCT-antiCD20 to Ramos CD20-expressing cells was also visualized by immunofluorescence. Cells were stained for CD20 (e.g., on Ramos cells), HA (a tag on aCD20-RCTs and control RCT-HA-GPA), and DNA (using DAPI). A co-culture of CD20+ Ramos cells and RCT-antiCD20 showed extensive colocalization of CD20 and HA, indicating the RCTs were binding the CD20+ cells and inducing hyper-crosslinking of CD20 (data not shown). In contrast, a co-culture of CD20+ Ramos cells and control RCT-HA-GPA showed minimal colocalization of CD20 and HA.

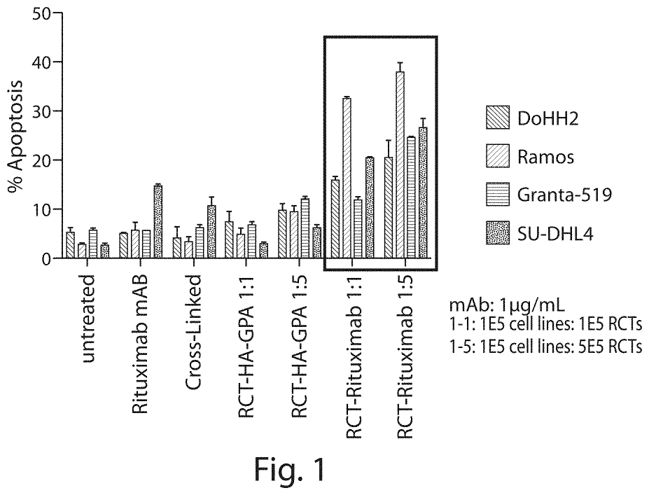

It was further assessed whether the interaction between RCT-antiCD20 and CD20-expressing cells could induce apoptosis, by co-culturing RCT-antiCD20 cells with a panel of CD20+ human lymphoma cell lines, representing a variety of lymphoma subtypes; DoHH2 (follicular lymphoma), Ramos (Burkitt's lymphoma), Granta-519 (Mantle Cell Lymphoma) and SU-DHL-4 (diffuse large B cell lymphoma). In all cases, RCT-antiCD20 co-culture resulted in increased apoptosis relative to RCT-HA-GPA or soluble Rituximab monoclonal antibody alone (FIG. 1). While not wishing to be bound by theory, direct tumor cell killing in vitro was hypothesized to be more effective than monoclonal antibody alone due to the hyper-crosslinking of CD20 on the lymphoma cell.

The performance of RCT-antiCD20 relative to soluble rituximab was quantified. In the apoptosis experiment described above, the number of anti-CD20 molecules on the RCT-antiCD20 was measured by quantitative flow cytometry. In samples with 1:1 ratio of RCT-antiCD20 to lymphoma cells, there were approximately 11.6-fold fewer molecules of anti-CD20 on the RCT than in the soluble antibody condition, despite similar levels of induced apoptosis. In samples with 1:5 ratio of RCT-antiCD20 to lymphoma cells, there were approximately 2.3-fold fewer molecules of anti-CD20 on the RCT than in the soluble antibody condition, despite much stronger induction of apoptosis by RCT-antiCD20. Thus, even though the soluble antibody was present at markedly higher levels than the erythroid cell-mounted antibody, the erythroid cells showed a much stronger effect.

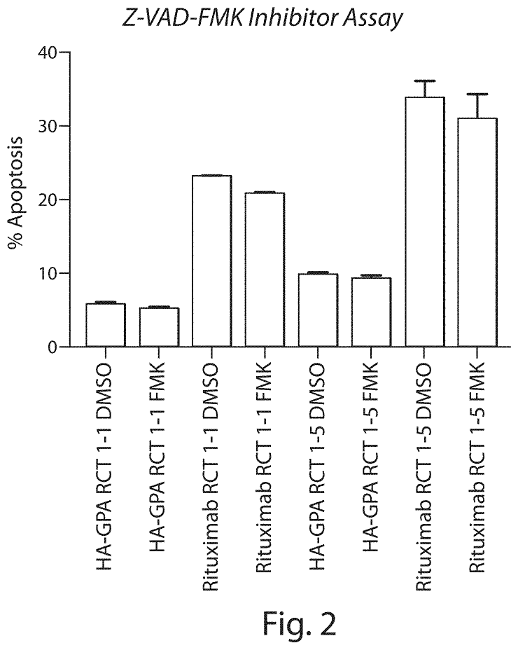

Rituximab is thought to kill CD20+ cells via caspase-mediated apoptosis, so this pathway was examined in CD20+ cells treated with anti-CD20 antibody alone or RCT-antiCD20. RCT-antiCD20 cells or control RCT-HA-GPA were added to CD20-expressing target cells. Co-cultures were treated with Z-VAD-FMK, an inhibitor of caspase-mediated apoptosis, or DMSO as a control. Apoptosis of the target cells was measured. As shown in FIG. 2, RCT-antiCD20 robustly promoted apoptosis in Ramos cells even in the presence of the inhibitor of caspase-mediated apoptosis. This surprising result indicates that RCT-antiCD20 kills CD20+ cancer cells through a mechanism different from that of the corresponding antibody alone. The result implies that in patients who are relapsed or refractory to an antibody such as an anti-CD20 antibody, e.g., due to an impaired caspase-dependent apoptosis pathway, can nevertheless be sensitive to that antibody presented on an erythrocyte.

To further characterize the mechanism of RCT-antiCD20 triggered apoptosis, BCL-xL and BCL-2 levels were studied. RCT-antiCD20 or control RCT-HA-GPA cells were cultured with each of three cancer cell lines (Raji, Ramos, or DoHH2). The surface levels of BCL-xL and BCL-2 on the cancer cells were measured by flow cytometry. In each line, RCT-antiCD20 but not control RCT-HA-GPA reduced BCL-xL and BCL-2 levels (see Table 7). Inhibition of the BCL2 and BCLxl anti-apoptotic pathways was dose dependent.

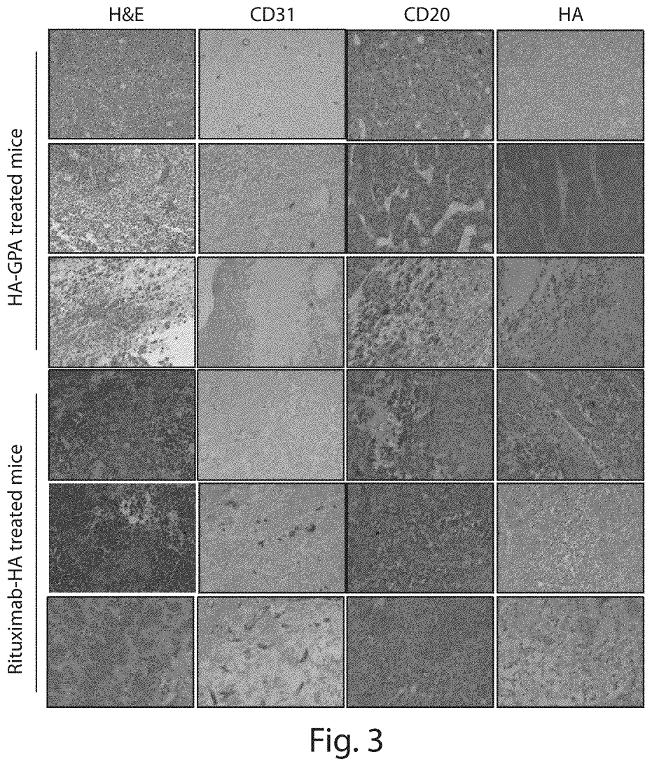

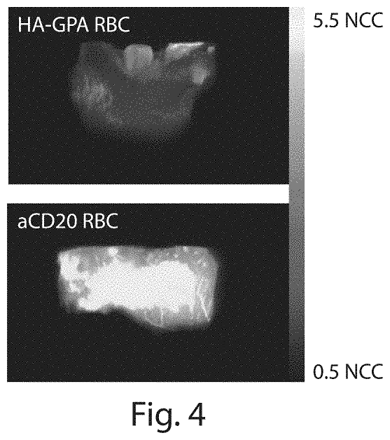

RCT-antiCD20 was also tested for ability to penetrate solid tumors. Because an erythroid cell is much larger than an antibody, it was not expected that an erythroid cell would be able to efficiently penetrate a solid tumor. (See, e.g., Newick et al. DOI: 10.1146/annurev-med-062315-120245, Thurber et al. doi: 10.1016/j.addr.2008.04.012) Xenograft tumors were formed by subcutaneous injection of Ramos cells into immunodeficient mice. The mice were intravenously administered mRBC-antiCD20 or control mRBC-HA-GPA. The mRBC-antiCD20 showed extensive tumor penetration, e.g., by H&E staining and HA staining, compared to mRBC-HA-GPA (FIG. 3). In particular, the mRBC-antiCD20 entered non-necrotic regions of the tumor, whereas mRBC-HA-GPA was generally restricted to the vasculature or necrotic regions of the tumor (FIG. 3). Specific tumor penetration was also shown using fluorescently labeled RCT-antiCD20 in comparison to control mRBC-HA-GPA (FIG. 4). The mean fluorescent intensity of mRBC-antiCD20 inside the tumor was more than twice as high as the control mRBC-HA-GPA. These data support the therapeutic use of erythroid cells comprising a tumor-binding moiety such as an anti-CD20 antibody. They also support the use of a tumor-binding moiety such as an anti-CD20 antibody as a targeting tool to direct the erythroid cells into tumors, e.g., CD20 positive tumors.

### Example 2: Erythroid Cells Expressing Immune Checkpoint Inhibitors Prevent Checkpoint Inhibition of T Cells

Enucleated erythroid cells were produced which express on their surface a fusion protein comprising, from N-to-C terminus, an scFv antibody domain, an epitope tag (either HA or Flag), and full-length GPA (extracellular, transmembrane, and cytoplasmic domains. The scFv domains were specific binders for PD-1 (RCT-antiPD1), PD-L1 (RCT-antiPDL1), or CTLA4 (RCT-antiCTLA4). The anti-PD-1 antibody domain is pembrolizumab-based. The anti-PD-L1 antibody domain is atezolizumab-based. The anti-CTLA4 antibody domain is ipilimumab-based.

Robust expression of anti-PD-L1 and anti-CTLA4 polypeptides was observed in a flow cytometry assay, with over 95.7% of cells expressing anti-PD-L1 after transfection with a vector encoding anti-PD-L1. Similarly, over 95.2% of cells expressed anti-CTLA4 after transfection with a vector encoding anti-CTLA4.

The cells were tested in a Jurkat/IL-2 assay. Jurkat T lymphocytes typically secrete IL-2, and also express PD-1, PD-L1, and CTLA4 upon stimulation. Co-culture of the Jurkat cells with NHL cells (Z138) triggers the immune checkpoint through PD-1, PD-L1, and CTLA4 signalling, resulting in downregulation of the Jurkat cells IL-2 secretion. A test agent, such as a RCT, is added to the co-culture. The test agent's ability to inhibit the immune checkpoint, thereby preserving T cell activity, is detected by the maintenance of high IL-2 secretion levels.

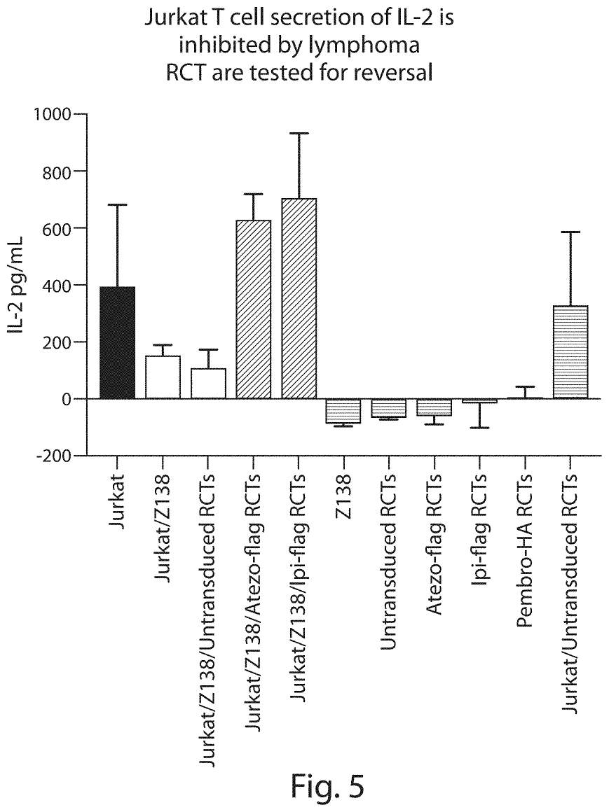

A 60% inhibition in IL-2 secretion upon co-culture with Z-138 (NHL) and Jurkat cells was observed in the Jurkat IL-2 assay (FIG. 5). Untransduced control RCT do not rescue the inhibitory effect. However, RCT-antiPDL1 showed a rescue and restoration of T cell IL-2 secretion, as did RCT-antiCTLA4 (FIG. 5). This experiment indicates that an RCT expressing an immune checkpoint inhibitor can promote an immune response by preventing checkpoint inhibition of T cells.

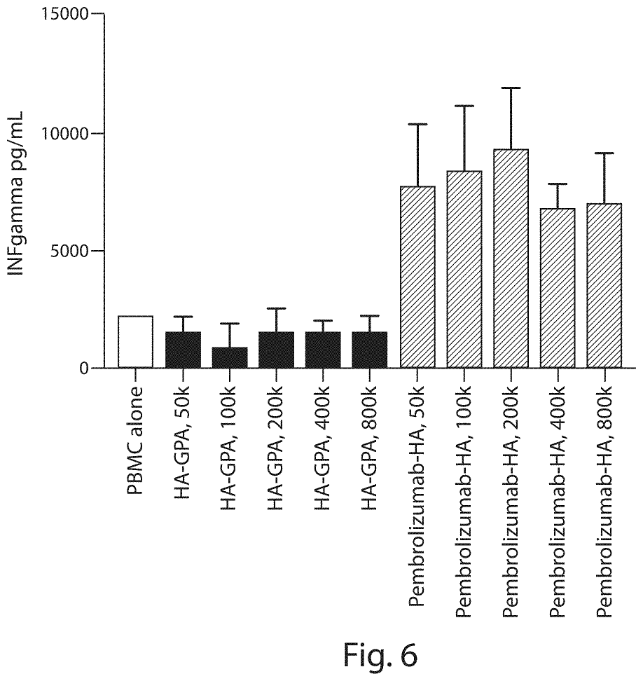

The ability of RCT-antiPD1 and RCT-antiPDL1 to elicit activation in a standard antigen recall assay was assayed. PBMC from a CMV positive donor were stimulated with CMV peptide. Memory T cells sensitive to immune checkpoint inhibition were tested for activation and gamma interferon secretin by co-culture with RCT-antiPD-1 or RCT-antiPD-L1 in comparison to control PBMCs or control RCT. A 3-4 fold increase was demonstrated in interferon-gamma secretion of peripheral blood mononuclear cells (PBMC) in an antigen recall assay (FIG. 6). Control RCT-HA-GPA did not increase interferon-gamma levels. RCT-antiPDL1 showed a 3-6 fold increase in interferon-gamma secretion in this assay (data not shown). This experiment indicates that an RCT expressing an immune checkpoint inhibitor can promote an immune response by preventing checkpoint inhibition of T cells.

RCT-antiPDL1 were tested for the ability to promote PBMC proliferation. Co-culture of PBMC with RCT-antiPDL1 resulted in a 3.6-fold increase in PBMC cells compared to PBMC alone. Co-culture of PBMC with control RCT-HA-GPA resulted in only a 2.3-fold increase. This experiment indicates an addition mechanism by which an RCT expressing an immune checkpoint inhibitor can promote the immune response.

### Example 3: Erythroid Cells Expressing Costimulatory Molecules Promote T Cell Activity

Enucleated erythroid cells were produced which express on their surface a fusion protein comprising, from N-to-C terminus, a 4-1BBL domain, an epitope tag (HA or Flag), and full-length GPA (extracellular, transmembrane, and cytoplasmic domains) (RCT-41BBL). 41-BB-L is a co-stimulatory protein that is expressed on antigen presenting cells and binds the 41-BB receptor on T-cells.

Binding of RCT-41-BB-L to recombinant 41-BB was determined using flow cytometry.

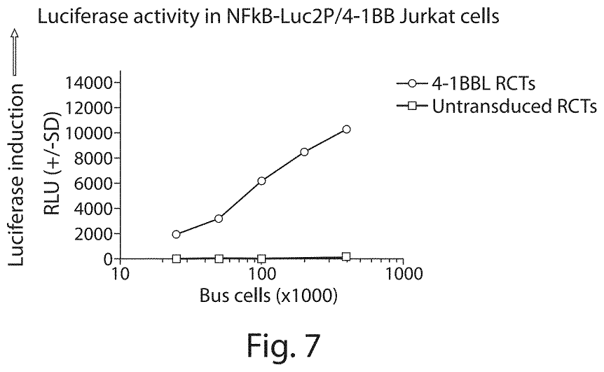

The RCT-41BBL were assayed by co-culture with Jurkat T cells overexpressing 4-1BB and NFkB-Luc2P (an NFκB-regulated luciferase reporter construct). Upon binding of 4-1BBL, T cells generally show elevated NFκB signalling, increased proliferation, increased secretion of IL-2 and interferon-gamma, and protection against activation induced cell death. In the assay, RCT-41BBL stimulated NFκB activation 30-fold compared with controls, as measured by luciferase activity (FIG. 7). Untransduced enucleated erythroid cells did not raise luciferase levels.

In addition, co-culture of PBMCs with RCT-41BBL showed a 1.7 fold increase in T-cell proliferation compared to PBMCs alone.

These experiments indicate that enucleated erythroid cells can successfully present a costimulatory molecule in a manner that promotes PBMC proliferation and activates a T cell intracellular signalling pathway responsible for immune cell activation.

### Example 4: Erythroid Cells Co-Expressing a Binding Agent to a Tumor Antigen and TRAIL Ligand Promote Apoptosis in Cancer Cells Expressing the Tumor Antigen

When erythroid cells were engineered to simultaneously express anti-CD20 as well as Trail ligand (an apoptosis inducing agent) using methods described above, co-culture of Ramos cells with RCT-antiCD20, RCT-Trail, and RCT-antiCD20+Trail (co-expressed) exert 32%, 47% and 76% apoptosis respectively after 48 hours, suggesting a synergistic effect of the co-expressing RCTs on tumor cell killing.

### Example 5: Erythroid Cells Expressing Costimulatory Molecules Promote T Cell Activity

Erythroid cells were generated that express different amounts of 4-1BB-L-HA by electroporation of increasing amounts of mRNA for 4-1BB-L-HA into RCT in the maturation phase, as described herein, as indicated in the table below. Greater amounts of electroporated mRNA led to greater expression of the encoded protein, both when measured as a percent of expressing cells and copies that are expressed per cell. Table 10 summarizes the expression level, based on 2 duplicates.

TABLE 10

| mRNA | % cell | Copies of |
|---|---|---|
| (ug/ | expressing | 4-1BB-L-HA |
| condition) | 4-1bB-L-HA | per cell |
| No EP | 0 | 0 |
| 0.025 | 74 | 42902 |
| 0.05 | 87.7 | 100500 |
| 0.1 | 91.75 | 274766 |
| 0.2 | 92 | 609145 |
| 0.4 | 90.6 | 874017 |
| 0.6 | 87.5 | 1015250 |

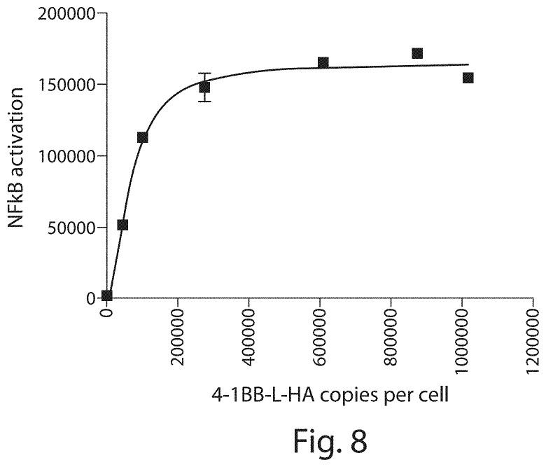

After electroporation the erythroid cells were tested for activity in a NFkB luciferase assay. In brief, erythroid cells were incubated with commercially available Jurkat cells that express 4-1BB and NFkB under the luciferase promoter (Promega). In this system, increased luciferase activity as measured by a commercially available kit (Promega) indicates activation of NFkB. 100,000 Jurkat 4-1BB NFkB/Luc cells were incubated with 50,000 RCT expressing different amounts of 4-1BB-L on the cell membrane. Cells were incubated for 6 hours, after which luciferase activity was measured. The data presented in FIG. 8 shows a robust increase in luciferase activity is initially dose dependent and then plateaus after 200,000 copies of 4-1BB-L that are expressed per cell. This data indicates that under these assay conditions, maximal luciferase activity is reached at about 200,000 copies of 4-1BB-L per cell.

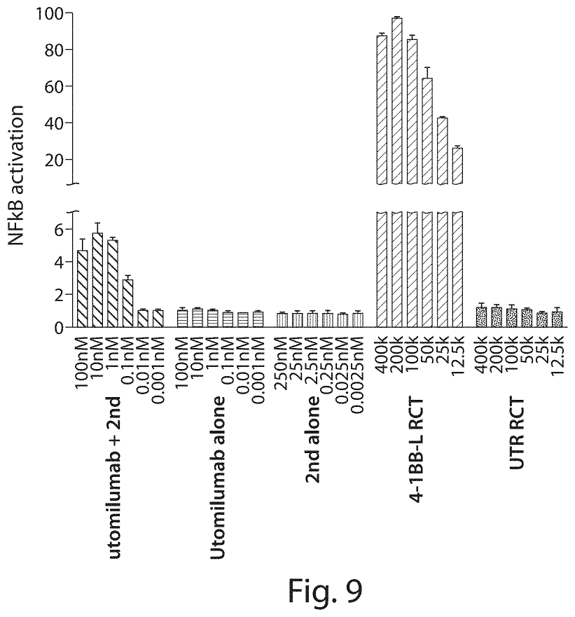

In another experiment, 4-1BB-L RCT were compared to the anti-4-1BB antibody Utomilumab for ability to promote T cell activity. 100,000 Jurkat 4-1BB NFkB/Luc cells were incubated with 4-1BB-L RCT or control RCT (un-transduced) at increasing cell numbers (12,500, 50,000, 100,000, 200,000, or 400,000). Jurkat 4-1BB NFkB/Luc cells were also incubated with increasing concentration of the 4-1BB agonist Utomilumab (concentration ranging from 0.001 nM to 100 nM) with or without a secondary anti human IgG that was used for cross linking of Utomilumab, at a concentration ranging from 0.0025 nM to 250 nM. The results indicate that Utomilumab alone did not increase NFkB activity in this assay, nor did the secondary antibody alone or the control RCT in any of the concentrations tested. High concentrations of Utomilumab (0.1 nM and above) that was crossed linked by a secondary anti human IgG antibody led to a 4-6 fold increase in luciferase activity, suggesting NFkB promoter activation. Incubation of the Jurkat 4-1BB NFkB/Luc cells with 4-1BB-L RCT induced a more robust activation of NFkB, starting at 12,500 RCT, that led to 20 fold NFkB activation (FIG. 9). Maximal activity was seen with 200,000 RCT incubated with the Jurkat 4-1BB NFkB/Luc cells, and led to 90-fold increase in luciferase activity and hence, NFkB activity. Thus, the erythroid cells expressing 4-1BBL lead to a dramatically higher (about 15-fold higher) induction of T cell activity compared to a cross-linked antibody. Without wishing to be bound by theory, trimerization of 4-1BBL at the erythroid cell surface may contribute to its high activity.

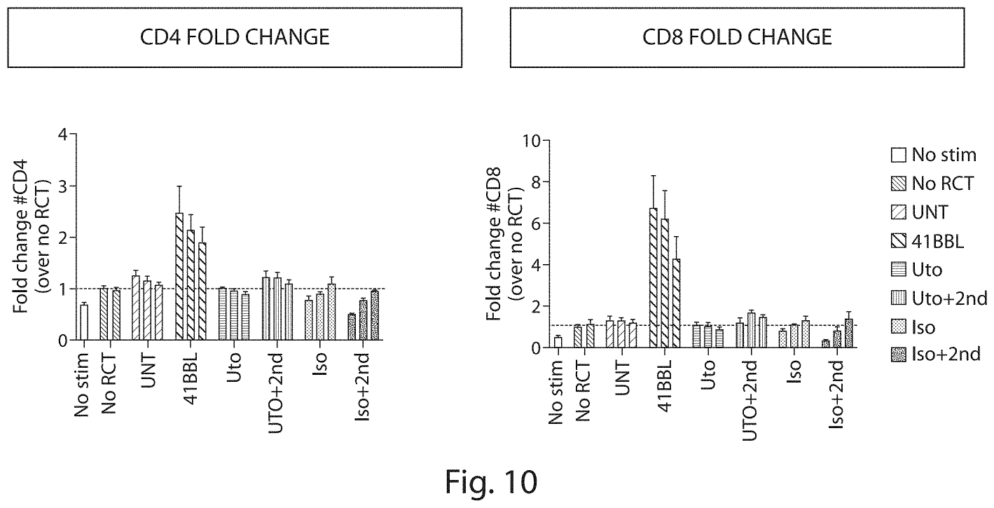

4-1BB-L RCT were also shown to increase PBMC proliferation. Human peripheral blood mononuclear cells (PBMC) from 3 different donors were obtained from Astrate biologics and were analyzed in a proliferation assay. PBMCs were labelled with Cell Trace Far Red (CTFR, Thermo-Fisher) according to the manufacturer protocol. 100000 CTFR labelled PBMCs were left untreated or stimulated with 0.5 ug/mL CD3 antibody. Cells were incubated with 12,500, 25,000 or 50,000 RCT (control or expressing 4-1BB-L), or increasing doses of Utomilumab (1 nM, 10 nM or 100 nM) or isotype control antibody, with or without a secondary anti human IgG antibody (2.5 nM, 25 nM or 250 nM). Cells were incubated for 5 days and analyzed in flow cytometry. As shown in FIG. 10, Utomilumab with cross linking antibody led to a small increase in proliferation of CD4 and CD8 T cells. On the other hand, incubation of CTFR PBMC with RCT 4-1BB-L led to a dose dependent 2-2.5 fold increase in CD4 proliferation, and dose dependent 4-7 fold increase in proliferation of CD8 cells. The data represents proliferation of PBMCs from 3 different human donors, and the calculation of fold increase in CD4 and CD8 proliferation was relative to the proliferation of the PBMCs from the same donor with CD3 antibody. This experiment indicates that RCT 4-1BB-L promotes proliferation of both CD4+ T cells and CD8+ T cells, with a more robust effect in CD8+ cells than CD4+ cells.

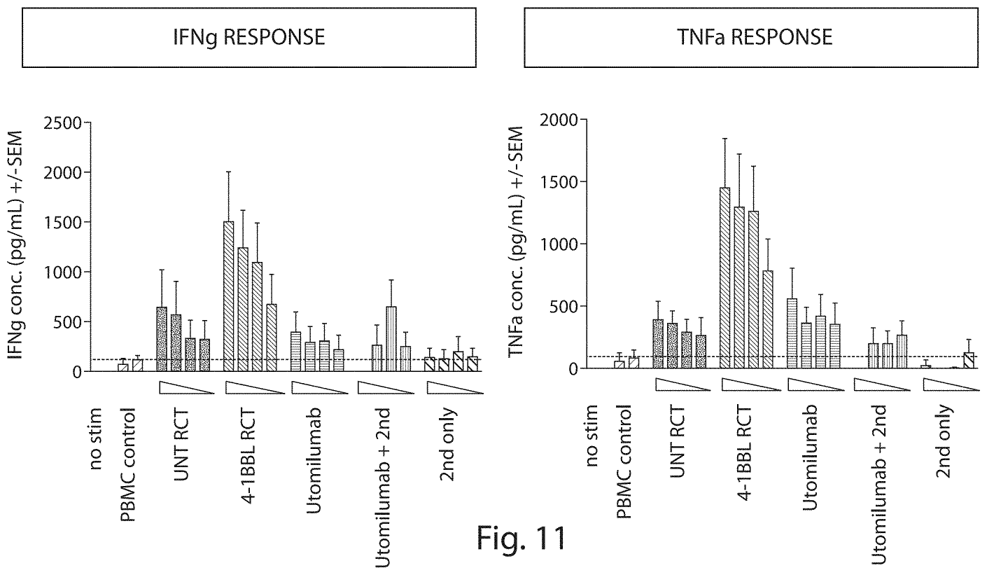

4-1BB-L RCT were also shown to increase cytokine secretion. Human PBMC from 3 different donors were obtained from Astrate biologics and were analyzed in a cytokine secretion assay. 100,000 PBMCs were left untreated or stimulated with 0.5 ug/mL CD3 antibody. Cells were incubated with 12,500, 25,000, 50,000 or 100,000 RCT (control or 4-1BB-L), or increasing doses of Utomilumab (1 nM, 10 nM, 100 nM or 1 uM) or isotype control antibody, with or without a secondary anti human IgG antibody (2.5 nM, 25 nM, 250 nM or 2.5 uM). Cells were incubated for 5 days and analyzed for cytokine secretion in flow cytometry based cytokine panel (Biolegend legendplex human inflammation panel) according to the manufacturer's protocol. Data presented in FIG. 11 indicates that Utomilumab alone led to a maximal secretion of 400 pg/mL of IFNg and 600 pg/mL with the secondary antibody cross-linking, while incubation of PBMC with RCT-4-1bB-L led to 1500 pg/mL of IFNg. A similar effect is seen with TNFa secretion, where incubation of PBMCs with RCT-4-1BB-L led a robust increase in TNFa secretion that is not seen either with Utomilumab alone or with Utomilumab and secondary antibody.

### Example 6: Erythroid Cells Comprising 41BBL Slow Tumor Growth In Vivo

An MC38 mouse model system for colon cancer was used to test the effects of erythroid cells comprising 4-1BBL on tumor growth. Without being bound by theory, 41BBL is thought to slow tumor growth by eliciting diverse immune effector responses on both the innate and adaptive immune arms. The most potent responses stimulate CD8+ cytotoxic T cells to proliferate and increase their effector potential through increased interferon gamma production and expression of multiple granzymes. In contrast, published preclinical data using multiple 4-1BB agonists have shown little or no single agent antitumor activity in the MC38 or other models (Chen, et al, 2014; Kudo-Saito, et al, 2006; Kocak, et al, Canc Res 2006; Tirapu, et al, Int J Cancer 2004; John, et al, Canc Res 2012).

Cells were conjugated with 4-1BBL such that 94.7% of the cells were labelled with m4-1BB-L. The amount of molecules labelled per cell was quantified using flow cytometry. For dosing animals, there were an average of 1.1e9 m4-1BB-L mRBCs administered per dose with an average of 36,200 m4-1BB-L molecules per cell corresponding to 0.084 mg/kg m4-1BB-L per dose.

Fourteen female C57/B6 aged 6-8 weeks mice were inoculated s.c. in left flank with 5×10⁵ MC-38 cells. Animals' weights and condition were recorded daily, and tumors were measured 3 times per week.

Tumors were measured three times a week by measuring each tumor in 2 dimensions. Tumor volumes were calculated using the standard formula: (L×W²)/2. The mean tumor weight and standard error of the mean was calculated for each group at each time point.

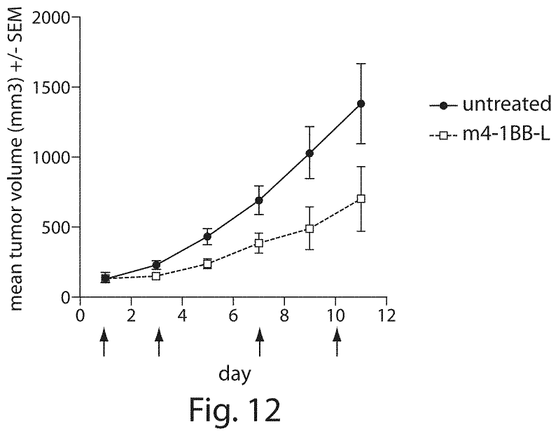

The observed anti-tumor activity of m4-1BB-L mRBC compared to untreated controls is shown in FIG. 12 and demonstrates a reduction in tumor growth in mice treated with m4-1BB-L mRBC. Tumor volume distributions demonstrated statistically significant differences between the groups as early as day 5 of the study and up until day 9 (P<0.05, T test).

Body weight was recorded daily. Changes in body weight were calculated for each mouse relatively to the body weight recorded on day 1. Treatment was well tolerated as indicated by overall increase in body weight for most mice. Mice that showed some decrease in body weight, did not lose more than 5% of the total body weight throughout the study.

These data support significant efficacy and potency of cellular presentation of 4-1BB-L via erythroid cells.

### Example 7: Erythroid Cells Expressing an Exogenous Polypeptide Comprising a Binding Agent to a Tumor Antigen Penetrate Solid Tumors

Enucleated erythroid cells were conjugated with anti-PD-L1 at their surface and tested for the ability to infiltrate tumors in mice.

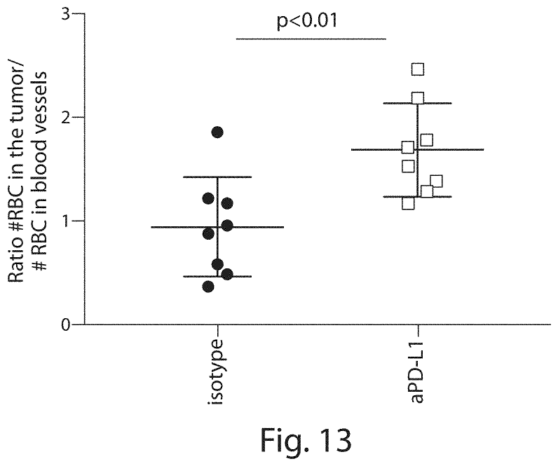

Mice were inoculated with B16F10 cells SC. Tumors were allowed to grow to 400 cubic mm before dosing. Murine RBC were conjugated with fragments antibody (Fab) from anti murine PD-L1 and isotype control. Conjugated murine RBC were labelled with CTFR according to the manufacturer's protocol. Cells were infused into the animals. One day after infusion, tumors were collected. Tumors were sectioned and stained with anti CD31 to visualize tumor vasculature and DAPI to visualize nuclei. Stained sections were scanned and pictures were taken. Using Halo software, the tumor areas and vasculature areas were identified. Total cell counts of labelled RBC in these two areas were taken for both isotype control and anti-PD-L1. The ratio between the RBC found in the tumor and the RBC found in the vessels was calculated. The data presented in FIG. 13 suggests that the ratio between RBC in the vessels and RBC in the tumor is 1 (average of measurement in tumors from 8 mice) for the isotype control conjugated RBC, indicating similar amounts in the tumor and the vasculature. The ratio between RBC in the vessel and RBC in the tumor is 1.7 for the anti PD-L1 treated mice, indicating enrichment of RBC in the tumor in the anti-PD-L1 group in comparison with the isotype control mice. The difference in ratio between the 2 groups was statistically significant with P<0.01 (student T test).

While not wishing to be bound by theory, tumors expressing higher levels of PD-L1 may respond better to RCTs comprising anti-PD-L1 than tumors that express lower levels of PD-L1. The B16F10 cells expressed about 300,000 copies per cell of PD-L1 when stimulated with IFN-gamma at 10 ng/ul. In contrast, CT26 cells expressed about 150,000 copies per cell of PD-L1 and A20 cells expressed about 100,000 copies per cell of PD-L1 under the same conditions. PD-L1 copy number was measured using a Quantum Simply Cellular kit (Bangs Laboratories). Erythroid cells comprising an anti-PD-L1 antibody at their surface showed greater binding to the IFN-gamma treated B16F10 cells and CT26 than to the A20 cells, consistent with greater levels of PD-L1 expression on the tumor cells leading to increased binding of the erythroid cells to the tumor cells.

### Example 8: Erythroid Cells Expressing Two Agents Promote Greater Immune Effector Cell Stimulation than Either Polypeptide Expressed Alone

Human peripheral blood mononuclear cells (PBMC) from 1 donor were obtained from Astrate biologics and were analyzed in an IL-2 cytokine secretion assay. 500,000 PBMCs were stimulated with 5 ug/mL CD3 antibody. Cells were incubated with 500,000 RCT control cells or RCTs expressing anti-PD-L1 or 4-1BB-L. In parallel, a combination of 500,000 RCT-anti PD-L1 and 500,000 RCT expressing 4-1BB-L was tested. Also in parallel, 500,000 cells expressing both anti PD-L1 and 4-1BB-L were tested. After 24 hours, supernatant was collected and IL-2 ELISA was performed to evaluated IL-2 secretion to the media as a result of incubation with RCT-anti PD-L1 and RCT 4-1BB-L. RCT anti PD-L1 led to 4 fold increase in IL-2 secretion as compared to PBMCs alone. RCT 4-1BB-L led to a 10 fold increased secretion of 4-1BB-L, whereas the combination of RCT anti PD-L1 and RCT-4-1BB-L led to a 13 fold increase in IL-2 secretion. The same 13-fold increase was measured with anti PD-L1 and 4-1BB-L were co-expressed on RCT as when PD-L1 and 4-1BB-L were expressed in a non-overlapping population of cells; however the co-expressing cells achieved this effect at half the dose (500,000 cells) compared to the non-overlapping population of cells (1,000,000 cells total).

This experiment indicates that erythroid cells expressing two agents produced a greater immune effector cell stimulation (measured by cytokine secretion) than either polypeptide expressed alone. The two agents used here were a costimulatory molecule (4-1BBL) and an agent that binds an immune checkpoint molecule (anti-PD-L1). Furthermore, the effect was greater when the two agents were expressed on the same cell than when the two agents were expressed in different populations of cells.

### Example 9: RCT Expressing Anti-Cancer Agents Promote Signalling, Proliferation, Cytokine Secretion, and Antigen Recall

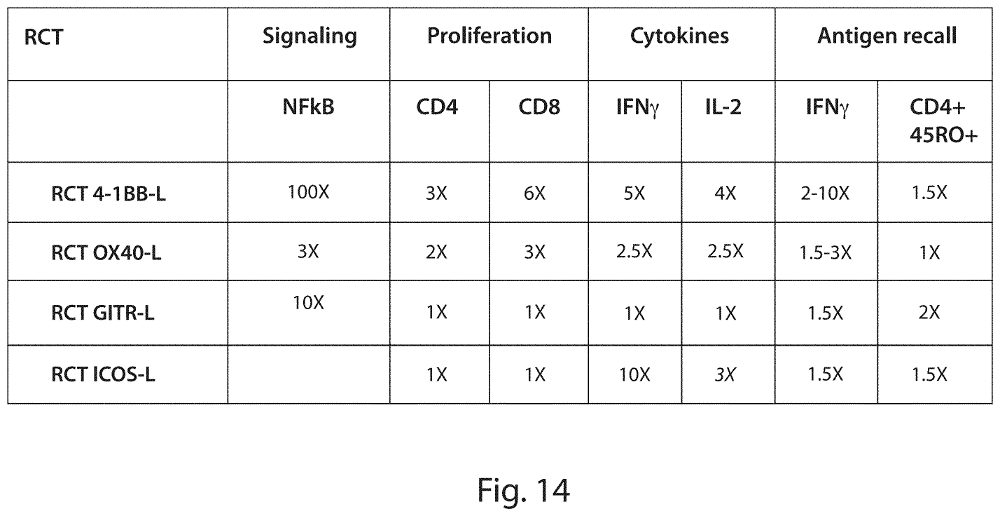

The Table of FIG. 14 summarizes data generated with RCT expressing 4-1BB-L, OX40-L, GITR-L, and ICOS-L. Signaling activity refers to an NFkB/Luc assay as described in Example 5 for 4-1BB-L, OX40-L and GITR-L (with Jurkat cells expressing NFkB/Luc and receptor for 4-1BB, OX40 or GITR respectively). The proliferation assay and cytokine secretion assays were performed as described in Example 5. The antigen recall assay was performed using 200,000 PBMCs from a CMV-positive donor (Astarte), which were stimulated using lysate of CMV-infected cells (Astarte) at 2 ug/mL. PBMCs were then incubated with 100,000 RCT expressing the indicated exogenous protein for 4 days. Cells were analyzed for the presence of memory T cells population with flow cytometry to detect cells that are CD4+ and CD45RO+. Supernatant from these experiments was collected and analyzed for IFNg using ELISA. These experiments indicate that erythroid cells expressing a variety of costimulatory molecules induce T cell activation.

1. A method of delivering a binding agent to an extravascular site in a subject comprising:
administering to the subject's bloodstream an effective amount of a preparation comprising a plurality of enucleated erythroid cells, wherein an enucleated erythroid cell of the plurality of enucleated erythroid cells comprises, on its surface, an exogenous polypeptide comprising a binding agent, wherein the binding agent comprises an antibody, an antibody fragment, a single-chain antibody, an scFv, or a nanobody; against an antigen present at an extravascular site,
thereby delivering the binding agent to the extravascular site.

2. A method of treating a subject having a resistant cancer comprising:
administering to the subject's bloodstream a preparation comprising a plurality of enucleated erythroid cells, wherein an enucleated erythroid cell of the plurality of enucleated erythroid cells comprises, on its surface, an exogenous polypeptide comprising a binding agent, wherein the binding agent comprises an antibody, an antibody fragment, a single-chain antibody, an scFv, or a nanobody against a tumor cell antigen, in an amount sufficient to treat the resistant cancer,
thereby treating the resistant cancer.

3. A method of treating a vascularized solid tumor in a subject comprising:
administering to the subject's bloodstream a preparation comprising a plurality of enucleated erythroid cells, wherein an enucleated erythroid cell of the plurality of enucleated erythroid cells comprises, on its surface, an exogenous polypeptide comprising a binding agent, wherein the binding agent comprises an antibody, an antibody fragment, a single-chain antibody, an scFv, or a nanobody against a tumor cell antigen, in an amount sufficient to treat the vascularized solid tumor, thereby treating the vascularized solid tumor.

4. The method of claim 1, wherein the exogenous polypeptide further comprises a transmembrane domain.

5. The method of claim 4, wherein the binding agent is an scFv.

6. The method of claim 4, wherein the binding agent is an anti-CD20 antibody, antibody fragment, single-chain antibody, scFv, or nanobody and the tumor antigen is CD20.

7. The method of claim 4, wherein the binding agent is an anti-PD-L1 antibody, antibody fragment, single-chain antibody, scFv, or nanobody and the antigen is PD-L1.

8. The method of claim 1, wherein the exogenous polypeptide is present at a copy number of greater than 10⁴.

9. The method of claim 1, wherein the enucleated erythroid cell further comprises a second exogenous polypeptide.

10. The method of claim 2, wherein the resistant cancer lacks a functional caspase apoptosis pathway.

11. The method of claim 2, wherein the resistant cancer comprises a mutation in a gene selected from the genes listed in Table 8.

12. The method of claim 2, wherein the resistant cancer is resistant to an antibody therapeutic listed in Table 1.

13. The method of claim 2, wherein the plurality of enucleated erythroid cells is administered in an amount and for a time effective to result in one or more of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of subject.

14. The method of claim 2, wherein the plurality of enucleated erythroid cells is administered in an amount and for a time effective to result in apoptosis of target resistant cancer cells.

15. The method of claim 2, wherein the binding agent is an anti-CD20 antibody, antibody fragment, single-chain antibody, scFv, or nanobody and the plurality of enucleated erythroid cells induces hypercrosslinking of CD20 on the surface of the resistant cancer cell.

16. The method of claim 2, wherein, following the administering step, the ratio of (i) the number of enucleated erythroid cells comprising the exogenous polypeptide resident in a tumor to (ii) the number of enucleated erythroid cells comprising the exogenous polypeptide in the subject's bloodstream is greater than 1:1.

17. The method of claim 2, wherein, following the administering step, the ratio of (i) the number of enucleated erythroid cells comprising the exogenous polypeptide to (ii) the number of endogenous erythroid cells in a tumor is at least 2:1.

18. The method of claim 2, wherein, following the administering step, the amount of binding agent resident in a tumor is at least 2-fold greater than the amount of an otherwise similar binding agent that is not associated with an erythroid cell.

19. The method of claim 2, wherein, following the administering step, the persistence of the enucleated erythroid cells comprising the exogenous polypeptide in a tumor is increased by at least 2-fold, as compared with an otherwise similar enucleated erythroid cell that lacks the binding agent.

20. The method of claim 2, wherein the subject is a human subject.


SEQUENCE LISTING (81 sequences)

SEQ ID NO: 1 - protein, 131 aa, Homo sapiens
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 2 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRRGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 4 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGIKINSWESSRRGHSFLSNLHLRNGELVIHEKGFYYIYSQTYFRFQEEIKENTKNDKQMVQYIYKYTDYPDPILLMKSARNSCWSKDAEYGLYSIYQGGIFELKENDRIFVSVTNEHLIDMDHEASFFGAFLVG

SEQ ID NO: 5 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 6 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQERIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 7 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 451 aa, Artificial Sequence
  Description of Artificial Sequence Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 211 aa, Artificial Sequence
  Description of Artificial Sequence Synthetic polypeptide
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 11 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 451 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 222 aa, Artificial Sequence
  Description of Artificial Sequence Synthetic polypeptide
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVP

SEQ ID NO: 14 - protein, 219 aa, Artificial Sequence
  Description of Artificial Sequence Synthetic polypeptide
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITREVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 453 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 450 aa, Artificial Sequence
  Description of Artificial Sequence Synthetic polypeptide
EVQLQQSGPELEKPGASVKLSCKASGYSFTGYNMNWVKQSHGKSLEWIGHIDPYYGDTSYNQKFRGKATLTVDKSSSTAYMQLKSLTSEDSAVYYCVKGGYYGHWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 208 aa, Artificial Sequence
  Description of Artificial Sequence Synthetic polypeptide
DIQMTQSPSSLSASLGERVSLTCRASQDIGSSLNWLQQGPDGTIKRLIYATSSLDSGVPKRFSGSRSGSDYSLTISSLESEDFVDYYCLQYVSSPPTFGAGTKLELKRADAAPSVFIFPPSDEQLKSGTASVVCLLNNFYPREAKVQWKVDNALQSGNSQESVTEQDSKDSTYSLSKADYEKHKVYACEVTHQGLSSPVTKSFNRGEC

SEQ ID NO: 21 - protein, 106 aa, Artificial Sequence
  Description of Artificial Sequence Synthetic polypeptide
QIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELK

SEQ ID NO: 22 - protein, 107 aa, Artificial Sequence
  Description of Artificial Sequence Synthetic polypeptide
DIQMTQSPSTLSASVGDRVTITCRASQSINTWLAWYQQKPGKAPKLLMYKASSLESGVPSRFIGSGSGTEFTLTISSLQPDDFATYYCQQYNSDSKMFGQGTKVEVK

SEQ ID NO: 23 - protein, 116 aa, Artificial Sequence
  Description of Artificial Sequence Synthetic polypeptide
QVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSS

SEQ ID NO: 24 - protein, 117 aa, Artificial Sequence [sequence omitted]

SEQ ID NO: 25 - protein, 443 aa, Artificial Sequence
  Description of Artificial Sequence Synthetic polypeptide
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 26 - protein, 209 aa, Artificial Sequence
  Description of Artificial Sequence Synthetic polypeptide
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 27 - protein, 447 aa, Artificial Sequence
  Description of Artificial Sequence Synthetic polypeptide
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 210 aa, Artificial Sequence
  Description of Artificial Sequence Synthetic polypeptide
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 29 - protein, 447 aa, Artificial Sequence; identical to SEQ ID NO: 27
  Description of Artificial Sequence Synthetic polypeptide

SEQ ID NO: 30 - protein, 210 aa, Artificial Sequence; identical to SEQ ID NO: 28
  Description of Artificial Sequence Synthetic polypeptide

SEQ ID NO: 31 - protein, 221 aa, Artificial Sequence
  Description of Artificial Sequence Synthetic polypeptide
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 32 - protein, 214 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 222 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 34 - protein, 211 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 35 - protein, 448 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 215 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 446 aa, Artificial Sequence
  Description of Artificial Sequence Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 218 aa, Artificial Sequence
  Description of Artificial Sequence Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 448 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 219 aa, Artificial Sequence
  Description of Artificial Sequence Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 449 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 451 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 215 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 440 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 218 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 457 aa, Artificial Sequence
  Description of Artificial Sequence Synthetic polypeptide
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 449 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, Artificial Sequence
  Description of Artificial Sequence Synthetic polypeptide
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 451 aa, Artificial Sequence
  Description of Artificial Sequence Synthetic polypeptide
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 451 aa, Artificial Sequence; identical to SEQ ID NO: 55
  Description of Artificial Sequence Synthetic polypeptide

SEQ ID NO: 57 - protein, 214 aa, Artificial Sequence
  source: Description of Artificial Sequence Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 214 aa, Artificial Sequence; identical to SEQ ID NO: 57
  Description of Artificial Sequence Synthetic polypeptide

SEQ ID NO: 59 - protein, 452 aa, Artificial Sequence
  Description of Artificial Sequence Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 446 aa, Homo sapiens
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, Homo sapiens
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 443 aa, Artificial Sequence
  Description of Artificial Sequence Synthetic polypeptide
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 220 aa, Artificial Sequence
  Description of Artificial Sequence Synthetic polypeptide
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 443 aa, Artificial Sequence; identical to SEQ ID NO: 63
  Description of Artificial Sequence Synthetic polypeptide

SEQ ID NO: 66 - protein, 220 aa, Artificial Sequence; identical to SEQ ID NO: 64
  Description of Artificial Sequence Synthetic polypeptide

SEQ ID NO: 67 - protein, 504 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 68 - protein, 449 aa, Artificial Sequence
  Description of Artificial Sequence Synthetic polypeptide
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 213 aa, Artificial Sequence
  Description of Artificial Sequence Synthetic polypeptide
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 452 aa, Artificial Sequence
  Description of Artificial Sequence Synthetic polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 215 aa, Artificial Sequence
  Description of Artificial Sequence Synthetic polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 447 aa, Artificial Sequence
  Description of Artificial Sequence Synthetic polypeptide
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 216 aa, Artificial Sequence
  Description of Artificial Sequence Synthetic polypeptide
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 74 - protein, 450 aa, Artificial Sequence
  Description of Artificial Sequence Synthetic polypeptide
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 214 aa, Artificial Sequence
  Description of Artificial Sequence Synthetic polypeptide
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 447 aa, Artificial Sequence
  Description of Artificial Sequence Synthetic polypeptide
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 213 aa, Artificial Sequence
  Description of Artificial Sequence Synthetic polypeptide
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 80 - protein, 205 aa, Artificial Sequence
  Description of Artificial Sequence Synthetic polypeptide
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 81 - protein, 238 aa, Artificial Sequence
  Description of Artificial Sequence Synthetic polypeptide
VQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK